SEQUENCE LISTING (33 sequences)

SEQ ID NO: 1 - DNA, 1256 bp, Staphylococcus aureus
  CDS (483)..(1103)
tagcaataccttttcctctagctgaagcatcgacataaatagaatgttcgattgtatataggtatgctggccaaggtctaaatgaaccgaacgtcgcaaaccctaagacacttccattttcctcaaatacaaagataggctcatgcttacgttgtttcgtttcaaaccatgcgacacgttcgtctatggtttgtggttcataagtataaacagctgtagtattgataatggcatcattgtatatcgctaatatagcgtttaaatcctcttttttagcgtatctaatcatatcaattcccccttagtaattattaaaagcgtttcgttatttgaatgcaaatatgtgtaatgaaatctaacgtaaaagtatacatgtaaattttatagtataaaatgaattgctatgagtcattttgaaattaatggtatactatatgaaatgttaacaggcattgtgaaatgtataaaaggagccttaacgtatgaaaaaatggacaaatcgattaatgacaatcgctggtgtggtacttatcctagtggcagcatatttgtttgctaaaccacatatcgataattatcttcacgataaagataaagatgaaaagattgaacaatatgataaaaatgtaaaagaacaggcgagtaaagataaaaagcagcaagctaaacctcaaattccgaaagataaatcgaaagtggcaggctatattgaaattccagatgctgatattaaagaaccagtatatccaggaccagcaacacctgaacaattaaatagaggtgtaagctttgcagaagaaaatgaatcactagatgatcaaaatatttcaattgcaggacacactttcattgaccgtccgaactatcaatttacaaatcttaaagcagccaaaaaaggtagtatggtgtactttaaagttggtaatgaaacacgtaagtataaaatgacaagtataagagatgttaagcctacagatgtaggagttctagatgaacaaaaaggtaaagataaacaattaacattaattacttgtgatgattacaatgaaaagacaggcgtttgggaaaaacgtaaaatctttgtagctacagaagtcaaataatctattacgctaatggatgaatatattgagtggaaaacagtcttgattgcgagactgttttttgtttggtatgaggtagcaatgacgacgtgtcattggtggagattgtaaaaatacataataaaaagaagcggcaatgtataccgctccttt

SEQ ID NO: 2 - protein, 206 aa, Staphylococcus aureus
MKKWTNRLMTIAGVVLILVAAYLFAKPHIDNYLHDKDKDEKIEQYDKNVKEQASKDKKQQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVK

SEQ ID NO: 3 - DNA, 2732 bp, Staphylococcus aureus
aaaaacccttgtggtgtcactgtacctgataaagattcagcaactttcatgtttatttcaaaaacttcttgcgcgtatgcgataatttgctgatctaatcttgccggttcaattgcaaataattgtgtaattacaattccactttgataagcttcttcaattaaatgcacaccttcaattaaagctaatccagttttatccctctcacgtttcttttttagcttgttcgcttgtttaattctattattttgtgcagaagtaatttgttccattgatagctcctcgctttatttttaaaaataaaaatattaatcattaataagatgaaaacatttgattgtatagttaatattaattaatcgcttttatcactcataatatttcaaattgtataaatttcttttatcgatactactactataaatcatacgccccaaaatatcattattaattcttttcttcttcaaaataaatcaaaatgatataattgatgattattttcaaagcacattcaaatcaaactatgttttagcaatttgttgttagcatgtttgtgttcattaaaaaaacgaccatcatcggtatcatgtatggtcgttacaaaagctaacaataccaattgtcataacaagtactgcaacctctttaaattcaattatttcatgtaactatagcctatatcatatgtaattactttgttatttataatcgggctactttcatcttcatttttacttctaacatgtttatgcgctgctttaaagacatcagattttaaccaatccgtaaaagcttgctttgatttccaaactgttaaaattttcacttcatcaaaatcttcttgttctaaagtttgtgtaacaaacatgccatcaaagccttctaatgtttcaatcccatgtctcgtgtaaaatcgttctataatatcttttgctgttccttttgttaacgtcagcctattttctgccataaatttcataattatcctcttttctgtttaacttaccttaattatttttgcgacaacaacaattcttttcgtcgtttcactatatgcatcttcgcacgttgataaagtcattattctatcttttaccgttacattaacatctgaattaattacagatttacgttttgtctcatctaaaaattgttgataatcttgatcattttcaaaatctgtacgtatgtaattatctttagtagtagttttatatgcactaaatacttgcaattgatatttaccatatttattgtcaaattcaattatcttgtgtttttcataaaacgattgctttaaataatcttctaacacatcaaacatcgtattatcaccgacatggtgcccgtataaaatagtattatgatttaaattcttcaattcatttctaaaatccataaaaatactacctttacgtcgatgttctcgctcaaaatctaaatttaaataatcgtgatttgtcttaccttgtagtactggataatttaatgatgttcctgataattttatccatccaacaatgtctttatttattttttcaagtgattcaaattgtggtctcacatgttcttgatgtttgctcatcagcatttgaaatttttgttgtaatttctcataatttgcgcgttcttgcttgtcttcaatatatgtttgaacaattttgtaaccaaaaatgataataattacaaccaataaaatttgtacaatagttaaaaatcgcttcattctcataaaaatcctcttttattaacgacgtttcttcagtcatcactaaaccagttgttgtaccgttttagattcgatttcgttgactttgacaaattaagtaaattagcattggaccaccgacaatcattaaaatagcattggctggaatttctaaaggaggctgtatcactcgtcctaataaatcagccactaacaatagccatgcaccaataactgtagaaaacggaataagtactctgtaattgcccccaactagctttctaaccacatgtggcacaataatacctaaaaaggctagttgtccaacaatcgcaacagttgcacttgctaaaaatactgctaataaacctgttaaccatctgtaacgatcaatattaaaaccgatacttcgcgcttgtatgtcgtctaaatttagtaaattcaatttaggggacaatagtaatgttaatattaatcccaataatgctgatactgctaatatgtatacgtcgctccatattttcattgttaagccttgaggaattttcattaaagggttttgagttaaaatttctaaaacaccatttaataatacgaataacgcaacacctactaatatcatacttacagcattgaatctaaatttagaatgcaacaatataattattaaaaatggtattaaacctccaataaaacttaataatggtaagtaaaagtacaattgtggaataaacaacatacaaagtgctctcattataagtgcacctgaggaaacgccaatgatattcgcctctgccaaaggattttgtagtgctgcttgtaataatgctccagaaactgctaacattgcgccaaccatcaatgcaattaatatacgtggcaatcgcaaatcaatgattgaatccactgcttcattgctaccagttgtaaattttgtaaataggtcattaaatgacaatttaattgtaccggttacaaacgaaatataagcagttgcgattaaaatgactaacaaacataaaaa

SEQ ID NO: 4 - protein, 244 aa, Staphylococcus aureus
MRMKRFLTIVQILLVVIIIIFGYKIVQTYIEDKQERANYEKLQQKFQMLMSKHQAHVRPQFESLEKINKDIVGWIKLSGTSLNYPVLQGKTNHDYLNLDFEREHRRKGSIFMDFRNELKILNHNTILYGHHVGDNTMFDVLEDYLKQSFYEKHKIIEFDNKYGKYQLQVFSAYKTTTKDNYIRTDFENDQDYQQFLDETKRKSVINSDVNVTVKDKIMTLSTCEDAYSETTKRIVVVAKIIKVS

SEQ ID NO: 5 - protein, 5 aa, Staphylococcus aureus
NPQTN

SEQ ID NO: 6 - protein, 5 aa, Staphylococcus aureus
  MOD_RES (3): X = anything
LPXXG

SEQ ID NO: 7 - protein, 35 aa, Staphylococcus aureus
LPETGEENPFIGTTVFGGLSLALGAALLAGRRREL

SEQ ID NO: 8 - protein, 37 aa, Staphylococcus aureus
LPETGGEESTNKGMLFGGLFSILGLALLRRNKKNHKA

SEQ ID NO: 9 - protein, 37 aa, Staphylococcus aureus
LPETGGEESTNNGMLFGGLFSILGLALLRRNKKNHKA

SEQ ID NO: 10 - protein, 38 aa, Staphylococcus aureus
LPDTGSEDEANTSLIWGLLASIGSLLLFRRKKENKDKK

SEQ ID NO: 11 - protein, 40 aa, Staphylococcus aureus
LPETGDKSENTNATLFGAMMALLGSLLLFRKRKQDHKEKA

SEQ ID NO: 12 - protein, 38 aa, Staphylococcus aureus
LPETGSENNNSNNGTLFGGLFAALGSLLSFGRRKKQNK

SEQ ID NO: 13 - protein, 38 aa, Staphylococcus aureus
LPETGNENSGSNNATLFGGLFAALGSLLLFGRRKKQNK

SEQ ID NO: 14 - protein, 38 aa, Staphylococcus aureus
LPETGSENNGSNNATLFGGLFAALGSLLLFGRRKKQNK

SEQ ID NO: 15 - protein, 40 aa, Staphylococcus aureus
LPDTGNDAQNNGTLFGSLFAALGGLFLVGRRRKNKNNEEK

SEQ ID NO: 16 - protein, 43 aa, Staphylococcus aureus
LPDTGDSIKQNGLLGGVMTLLVGLGLMKRKKKKDENDQDDSQA

SEQ ID NO: 17 - protein, 39 aa, Staphylococcus aureus
LPDTGMSHNDDLPYAELALGAGMAFLIRRFTKKDQQTEE

SEQ ID NO: 18 - protein, 32 aa, Staphylococcus aureus
LPNTGSEGMDLPLKEFALITGAALLARRRTKN

SEQ ID NO: 19 - protein, 37 aa, Staphylococcus aureus
LPAAGESMTSSILTASIAALLLVSGLFLAFRRRSTNK

SEQ ID NO: 20 - protein, 38 aa, Staphylococcus aureus
LPKTGLTSVDNFISTVAFATLALLGSLSLLLFKRKESK

SEQ ID NO: 21 - protein, 39 aa, Staphylococcus aureus
LPKAGETIKEHWLPISVIVGAMGVLMIWLSRRNKLKNKA

SEQ ID NO: 22 - protein, 33 aa, Staphylococcus aureus
LPKTGLESTQKGLIFSSIIGIAGLMLLARRRKN

SEQ ID NO: 23 - protein, 33 aa, Staphylococcus aureus; identical to SEQ ID NO: 22

SEQ ID NO: 24 - protein, 35 aa, Staphylococcus aureus
LPKTGETTSSQSWWGLYALLGMLALFIPKFRKESK

SEQ ID NO: 25 - protein, 36 aa, Staphylococcus aureus
LPQTGEESNKDMTLPLMALLALSSIVAFVLPRKRKN

SEQ ID NO: 26 - protein, 33 aa, Staphylococcus aureus
LPKTGMKIITSWITWVFIGILGLYLILRKRFNS

SEQ ID NO: 27 - protein, 34 aa, Staphylococcus pyogenes
LPLAGEVKSLLGILSIVLLGLLVLLYVKKLKSRL

SEQ ID NO: 28 - protein, 39 aa, Staphylococcus pyogenes
LPATGEKQHNMFFWMVTSCSLISSVFVISLKTKKRLSSC

SEQ ID NO: 29 - protein, 35 aa, Staphylococcus pyogenes
LPSTGEMVSYVSALGIVLVATITLYSIYKKLKTSK

SEQ ID NO: 30 - protein, 33 aa, Staphylococcus pyogenes
QVPTGVVGTLAPFAVLSIVAIGGVIYITKRKKA

SEQ ID NO: 31 - protein, 37 aa, Staphylococcus pyogenes
VPPTGLTTDGAIYLWLLLLVPFGLLVWLFGRKGLKND

SEQ ID NO: 32 - protein, 33 aa, Staphylococcus pyogenes
EVPTGVAMTVAPYIALGIVAVGGALYFVKKKNA

SEQ ID NO: 33 - protein, 39 aa, Staphylococcus aureus
NPQTNAGTPAYIYTIPVASLALLIAITLFVRKKSKGNVE

(19) United States
(12) Patent Application Publication
(10) Pub. No.: US 2006/0073530 A1
(43) Pub. Date: Apr. 6, 2006

(54) Methods and compositions involving sortase B

(76) Inventors: Olaf Schneewind, Chicago, IL (US); Sarkis N. Mazmanian, Brookline, MA (US); Kenneth Su, Irvine, CA (US); Hung Ton-That, Chicago, IL (US)

Correspondence Address: HELLER EHRMAN LLP, 275 MIDDLEFIELD ROAD, MENLO PARK, CA, 94025-3506, US

(21) Appl. No.: 10/219,783

(22) Filed: Aug. 15, 2002

Related U.S. Application Data
(60) Provisional application No. 60/312,738, filed on Aug. 15, 2001.

(51) Int. Cl. G01N 33/554; C12N 9/10; G01N 33/569

(52) U.S. Cl. 435/7.32; 435/193

(57) ABSTRACT

The present invention provides methods and compositions involving sortase-transamidases, including sortase B, and polypeptides that include a signal sorting sequence of NPQ/KTN/G. Methods of screening for inhibitors of Gram-positive bacteria as well as therapeutic, preventative, and research methods focusing on Gram-positive bacteria are also provided.

This Application claims the benefit under Title 35, United States Codes §119(e) of U.S. provisional application No. 60/312,738 filed on Aug. 15, 2001.

The government may own rights in the present invention pursuant to grant number A139987 from the National Institutes of Health.

### BACKGROUND OF THE INVENTION

1. Field of the Invention

The present invention relates generally to the field of bacteriology. More particularly, it concerns methods and compositions relating to sortase-transamidases, including sortase B, Gram-positive bacteria, and polypeptides containing a sorting signal with the motif NPQ/KTN/G cleaved by a sortase-transamidase.

2. Description of Related Art

Gram-positive bacteria that infect humans include Actinomyces, Bacillus, Enterococcus, Listeria, Myycobacterium, Staphlococcus, and Streptococcus. Infection by Gram-positive bacteria can range from a minor infection to a fatal infection. Moreover, some of these have become resistant to antiobiotics. Streptococcus agalactiae or group B streptococcus (GBS), for instance, is responsible for approximately 17,000 cases annually in the United States; approximately 7,500 occurred in newborns before recent prevention. Death occurs in 5% of infants and 16% of adults. Group A Streptococcus caused approximately 9,400 annual cases of invasive disease in 1999; of these, death resulted in approximately 10%-13%. Organ system failure and amputation also may result.

Moreover, in recent years some of these bacteria have become increasingly resistant to antiobiotics or some strains have been identified that are not susceptible to widely used antiobiotics. For example, Staphylococcus aureus, one of the most common causes of hospital- and community-acquired infections, includes strains that have become resistant to methicillin or that are not susceptible to vancomycin.

A need for other compositions and methods for preventing and treating microbial infection is clear, particularly with respect to Gram-positive bacteria. Surface proteins of Gram-positive bacteria play many important roles during the pathogenesis of human infections (Navarre et al., 1999). Staphylococcal protein A is linked to the cell wall peptidoglycan by a mechanism requiring a sorting signal that is composed of a LPXTG motif, a hydrophobic domain and a tail of mostly positively charged residues (Schneewind et al., 1995; Schneewind et al., 1993; Schneewind et al., 1992). After signal peptide-mediated initiation into the secretory pathway (Navarre et al., 1994), protein A precursors are retained in the envelope by a property of their C-terminal sorting signals (Navarre et al., 1996). The LPXTG motif is cleaved between the threonine (T) and the glycine (G) by sortase (SrtA) (Navarre et al., 1994), a transpeptidase that captures protein A as acyl enzyme intermediate at an active site cysteine moiety (Mazmanian et al., 1999; Ton-That et al., 1999). The nucleophilic attack of the amino group of peptidoglycan precursors resolves the acyl intermediate, resulting in amide linkage between the C-terminus of surface proteins and the peptidoglycan crossbridges (Navarre et al., 1998; Ton-That et al., 1997; Ton-That et al., 1999; Ton-That et al., 2000). Surface proteins linked to peptidoglycan precursors are incorporated into the envelope via the transpeptidation and transglycosylation reactions of cell wall synthesis (Ton-That et al., 1999).

Genome sequencing and homology searches revealed that Gram-positive pathogens encode at least two sortase genes, and in some cases, up to seven sortase genes (Mazmanian et al., 2001; Ton-That et al., 2001). While the sorting signal of LPXXG has been previously identified, many sorting signals remain unidentified. See PCT Publication Nos. WO 00/62804 and WO 99/09145, specifically incorporated by reference in their entireties.

Because of the emergence of antiobiotic resistant bacteria and bacteria not susceptible to antiobiotics, as well as the continued incidence of infection from Gram-positive bacteria in humans and other mammals, there is a continued need for the development of treatment and preventative options for bacterial infection. Also needed are screening methods to identify candidate agents for use as treatment or prevention.

### SUMMARY OF THE INVENTION

The present invention is based on the observation of another sorting signal in Gram-positive bacteria, as well as the observation that this sorting signal is recognized not by Srt A, but by another sortase-transamidase, Srt B. This observation provides another motif that can be employed in screening methods for antibiotics against Gram-positive bacteria, in expressing a polypeptide of interest on the surface of a bacteria, in treating or diagnosing a Gram-positive bacteria infection, in inducing an immune response against an antigen of interest, and in identifying additional sorting signals. The observations also provide compositions for use in the methods thereof.

In some embodiments of the invention, there are methods for screening for a sortase-transamidase inhibitor comprising: a) incubating at least a first sortase-transamidase and at least a first recombinant polypeptide comprising a sorting signal of NPQ/KTN/G at its carboxyl-terminal end, under conditions to allow the first sortase-transamidase to cleave the polypeptide within the sorting signal; b) incubating the first sortase-transamidase or the recombinant polypeptide with a candidate compound; and c) assaying for activity of the first sortase-transamidase, wherein a reduction in activity identifies the compound as a candidate inhibitor. One of ordinary skill in the art recognizes that NPQ/KTN/G represents the following amino acid sequences: NPQTN, NPKTN, NPQTG, and NPKTG. The invention is applicable to Gram-positive bacteria, including the following: Actinomyces, Bacillus, Bifidobacterium, Cellulomonas, Clostridium, Corynebacterium, Micrococcus, Mycobacterium, Nocardia, Staphylococcus, Streptococcus and Streptomyces, and their different species. However, it is also contemplated that methods of the invention, including those discussed below, may be employed a Gram-positive bacteria having a peptidoglycan. In some embodiments, the method employs a substitute for the peptidoglycan, such as a synthetic composition that mimics peptidoglycan so that sortase-transamidase activity can be demonstrated, such as by cleaving a sorting signal.

Screening methods may also include a comparison step in which the activity of the first sortase-transamidase is compared to the activity of a sortase-transamidase that was not incubated with the candidate compound. It is contemplated that in some embodiments, the sortase-transamidase used as a relative value of activity is from the same batch or preparation as the first sortase-transamidase. It is contemplated that the order the components are incubated with one another is not limited and that any combination is envisioned such that the sortase-transamidase, the polypeptide, and the candidate compound are ultimately incubated together. Moreover, it is contemplated that in embodiments that include a Gram-positive bacterium with a peptidoglycan, every order of incubation is contemplated. Thus, the order of the components is not limited in any method of the invention unless specified.

Furthermore, it is contemplated that in screening methods of the invention, a candidate compound may undergo more than one screening. A compound may be screened multiple times (i.e., more than once), including in duplicate screens and against different sortase-transamidases, different polypeptides with different sorting signals, and with different Gram-positive bacterium. “Different sortase-transamidases” refers to 1) the same sortase-transamidases from a different species; 2) different sortase-transamidases from the same species; 3) a different sortase-transamidase from a different species. Different sorting signals include sorting signals comprising NPQ/KTN/G or LPX₃X₄G. A candidate compound may be screened with respect to one sorting signal, for example, NPQ/KTN/G, and then screened with respect to LPX₃X₄G. Thus, additional steps of the screening method include: d) incubating the candidate substance with a second sortase-transamidase and a second recombinant polypeptide comprising a sorting signal of LPX₃X₄G at its carboxyl-terminal end, under conditions to allow the second sortase-transamidase to cleave the second polypeptide within the sorting signal; and e) assaying for activity of the second sortase-transamidase, wherein a reduction in activity identifies the compound as a candidate inhibitor.

In still further aspects of the invention, additional screening steps also include: d) incubating the candidate substance with a third sortase-transamidase and a third recombinant polypeptide comprising a sorting signal, under conditions to allow the third sortase-transamidase to cleave the third polypeptide within the sorting signal; and e) assaying for activity of the third sortase-transamidase, wherein a reduction in activity identifies the compound as a candidate inhibitor. It is contemplated that a candidate compound may be screened by this method 1, 2, 3, 4, 5, 6, 7, 8, 9, 10 or more times with the different iterations described above.

In methods and compositions of the invention, a sortase-transamidase is involved. It is contemplated that a sortase-transamidase is any sortase-transamidase from a Gram-negative bacteria, including SrtA, SrtB, SrtC, or any other sortase-transamidase, particularly those disclosed herein, known to one of ordinary skill in the art, or that utilizes a sorting signal comprising NPQ/KTN/G or LPX₃X₄G. In some embodiments, a sortase-transamidase from the Staphylococcus family is employed, such as S. aureus. In still further embodiments, a Srt B comprises SEQ ID NO:4. Alternatively, a Srt A comprising SEQ ID NO:2 is used.

Sortase-transamidases of the invention may be substantially purified. In other embodiments the sortase-transamidase is recombinant, meaning it has been generated using recombinant DNA technology at some point. In some embodiments, a sortase-transamidase may be encoded by SEQ ID NO:1, which encodes Srt A or encoded by SEQ ID NO:3, which encodes Srt B. In still further embodiments the sortase-transamidase is recombinant and substantially purified.

Polypeptides containing a sorting signal are employed in a number of methods of the invention. A “sorting signal” is an amino acid sequence that allows a proteinaceous compound to be recognized by a sortase-transamidase and cleaved within the signal by the sortase-transamidase. In the context of a Gram-positive bacteria with a peptidoglycan, a “sorting signal” allows a proteinaceous compound to be recognized by a sortase-transamidase, cleaved within the signal by the sortase-transamidase, and covalently bonded to the peptidoglycan. A “polypeptide of interest” refers to a polypeptide that has already been cleaved and that may be displayed on the surface of a Gram-positive bacterium. A polypeptide comprising a sorting signal may be rendered into a “polypeptide of interest” if exposed to the relevant sortase-transamidase.

Some embodiments of the invention include a polypeptide having a sorting signal of at least or at most 5, 6, 7, 8, 9, 10, 11, 12, 13, 14, 15, 16, 17, 18, 19, 20, or more contiguous amino acids from SEQ ID NO:5, SEQ ID NO:6; SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, or SEQ ID NO:33. A sorting signal with a NPQ/KTN/G motif may further include sequences from SEQ ID N:5 or SEQ ID NO:33. A sorting signal with a LPX₃X₄G motif may further include sequences from SEQ ID NO:6; SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, or SEQ ID NO:32. It is contemplated that more than one stretch (3 amino acids or more) from a SEQ ID NO may be included as a sorting signal in a polypeptide.

In still further embodiments, a polypeptide with a sorting signal with a LPX₃X₄G motif may further include substantially hydrophobic domain of at least 31 amino acids carboxyl to the motif and a charged tail region with at least two positively charged residues carboxyl to the substantially hydrophobic domain, wherein at least one of the positively charged residues is an arginine and the two positively charged residues are located at residues 31-33 from the motif. Also, while the NPQ/KTN/G motif is at the carboxyl end of a protein in some embodiments, in others, it, or any other sorting signal, is not and may be anywhere in a polypeptide.

High throughput screening methods are contemplated for use with the methods disclosed herein. In some cases, a non-reactive structure is employed. One of more components of the screening method may be attached to the structure.

In some aspects of the invention, a polypeptide with a sorting signal further includes a label or detection reagent, which may be radioactive, colorimetric, or enzymatic.

Some methods of the invention involve assaying for sortase-transamidase activity. This can be accomplished by a number of ways, including by measuring the amount of cleaved or uncleaved polypeptide or by measuring the amount of the polypeptide displayed or not displayed on the surface of the bacterium. In some embodiments, measurements involve using a proteinaceous compound that specifically binds the polypeptide of interest or the polypeptide comprising a sorting signal (before being cleaved). In other embodiments detection of a polypeptide of interest is a step. Such proteinaceous compounds may also be labeled or attached to a detection reagent, which can be radioactive, colorimetric, or enzymatic. The proteinaceous compound may also be an antibody, a ligand for the polypeptide of interest, or a substrate for the polypeptide.

In addition to in vitro screening, the invention provides in vivo screening. In vivo screening includes a) administering to an animal a Gram-positive bacterium; b) administering to the animal the candidate compound; c) assaying the pathogenicity of the bacterium. In vivo screening may be implemented alone or in conjunction with in vitro screening. Thus, a candidate compound that inhibits a sortase-transamidase in vitro may be tested in vivo. In some embodiments, the Gram-positive bacterium has a mutation in at least one sortase-transamidase-encoding nucleic acid, wherein the bacterium does not express a functional sortase-transamidase encoded by the nucleic acid. In other embodiments, the Gram-positive bacterium is Staphylococcus aureus. The method, in additional embodiments, further includes comparing the pathogenicity of the bacteria in the animal with a second animal administered a Gram-positive bacterium having a mutation in srt A, srt B, or both. It is contemplated that the animal is a mouse, such as a murine renal abscess model.

The candidate compound is administered to the animal before, during, or after administration of the bacterium to the animal. It is contemplated that the candidate compound may be given 1, 2, 3, 4, 5, 6, 7, 8, 9, 10, 11, 12 or more hours, 1, 2, 3, 4, 5 6, 7 or more days after the bacteria is administered.

In addition to screening for inhibitors, the invention includes other screening methods. In some embodiments, methods for screening for expression of a recombinant polypeptide are included. Such methods involve: a) incubating a sortase-transamidase, a Gram-positive bacterium having a peptidoglycan, and a recombinant polypeptide comprising a sorting signal of NPQ/KTN/G at its carboxyl-terminal end, under conditions to allow the sortase-transamidase to cleave the polypeptide within the sorting signal; and b) assaying for display of the polypeptide on the surface of the Gram-positive bacterium. Embodiments discussed with respect to screening for inhibitors are envisioned equally applicable to other screening methods, such as methods of screening for expression of a recombinant polypeptide.

Methods of treating or diagnosing a Gram-positive bacteria infection in an organism are also part of the invention. Such methods include: a) linking an antibiotic or a detection reagent to a polypeptide comprising a sorting signal of NPQ/KTN/G at its carboxyl-terminal end; and b) introducing the linked polypeptide to the organism, wherein the linked polypeptide becomes covalently cross-linked to the bacterium. In some embodiments, the antibiotic or detection reagent is conjugated to the polypeptide. Such methods also include identifying a patient in need of treatment for a Gram-positive bacteria infection. Thus, a patient suspected of having such a bacterial infection may be diagnosed or treated as described above.

Thus, the invention also includes a conjugate comprising an antibiotic or detection reagent conjugated to a polypeptide comprising a sorting signal of NPQ/KTN/G, which may be at its carboxyl-terminal end.

In still further embodiments is a recombinant cleaved polypeptide displayed on the surface of a Gram-positive bacterium comprising a sortase-transamidase by covalent linkage, wherein the polypeptide comprised an amino acid sequence of NPQ/KTN/G prior to being cleaved by the sortase-transamidase. In some instances the bacterium is Staphylococcus aureus.

The invention also concerns eliciting an immune response in a subject and compositions that may accomplish this purpose. Therefore, the invention includes a vaccine comprising a Gram-positive bacterium displaying a recombinant cleaved polypeptide or polypeptide of interest on its surface, wherein the polypeptide is covalently attached to the bacterium and comprises a sorting sequence comprising an amino acid sequence of NPQ/KTN/G or LPX₃X₄G prior to being cleaved by a sortase-transamidase in the bacterium. In some embodiments the polypeptide comprises a sorting sequence comprising an amino acid sequence of NPQ/KTN/G prior to being cleaved by a sortase-transamidase in the bacterium. Furthermore, a vaccine include a polypeptide that comprises 5 or more contiguous amino acids from SEQ ID NO:7, SEQ ID NO:8, SEQ ID NO:9, SEQ ID NO:10, SEQ ID NO:1, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, or SEQ ID NO:33. It is specifically contemplated that the Gram-positive bacterium is Staphylococcus, including S. aureus. In further aspects of the invention, the Gram-positive bacterium is attenuated with respect to any of a wild type bacterium's characteristics. The bacterium, in some embodiments, comprises a genetic mutation, wherein the mutation reduces the pathogenicity of the bacterium compared to a bacterium lacking the mutation.

As discussed above, the invention includes a method for inducing an immune response in an animal comprising administering to the animal vaccines of the invention an amount effective to induce an immune response against the antigen.

Additional embodiments are directed at a method of making a vaccine comprising incubating a sortase-transamidase, a Gram-positive bacterium having a peptidoglycan, and a recombinant polypeptide comprising a sorting signal of NPQ/KTN/G at its carboxyl-terminal end and an antigen, under conditions to allow the sortase-transamidase to cleave the polypeptide within the sorting signal. It is contemplated that the antigen can be anything against which an immune response is desired, including a tumor-associated antigen or a virus antigen or a bacterial antigen. The Gram-positive bacterium has, in some embodiments, a genetic mutation, wherein the mutation reduces the pathogenicity of the bacterium compared to a bacterium lacking the mutation.

Furthermore, methods for identifying a sorting signal constitute part of the invention. In one embodiment, the method comprises: a) searching a protein database for homologous amino acid sequences in Gram-positive bacterium using a query lacking the sequence of LPX₃X₄G and NPQTN; b) identifying at least a first protein sequence that has at least 10% identity or 20% homology to a second protein sequence; c) obtaining a polypeptide comprising an identified protein sequence; and d) incubating the polypeptide with a sortase-transamidase under conditions to allow the polypeptide to be cleaved.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Surface proteins bearing C-terminal sorting signals with an LPXTG motif are anchored to the cell wall in a srtA dependent manner. Surface proteins and their sorting signals were identified with Blast searches in the genome databases. Fusion of C-terminal sorting signals to the C-terminus of staphylococcal enterotoxin B (Seb) generates hybrid proteins that are substrate for cell wall sorting. Surface protein anchoring was measured in pulse labeling experiments as the percent amount of [³⁵S] methionine protein that was cleaved to generate the mature anchored species. ND, the ability of sorting signals to anchor fused Seb was not determined.

FIG. 2. Organization of the sasK/srtB operon. A six gene csitron is transcribed from a canonical promoter binding site. Down stream of the promoter is located a canonical fur binding site (conserved nucleotides of the fur box are highlighted in red underneath the drawing). The operon is comprised of the sasK gene (encoding a surface protein with an NPQTN type sorting signal), the samA gene (a type I membrane protein), the ficB gene (a lipoprotein and ABC transporter for ferrichromes), ficY (a ferrichrome permease membrane transporter), srtB (sortase B) and orf107 (a cytoplasmic polypeptide of unknown function). The cell wall sorting signal of SasK is described. The sasK/srtB operon was also found in B. halodurans and B. anthracis with a notable variation of the NPQTN sequence.

FIG. 3. SasK is anchored to the cell wall in a srtB dependent manner. SasK_{FLAG} contains a FLAG epitope tag inserted upstream of the C-terminal hydrophobic domain and can be detected with monoclonal ant-FLAG antibodies. Cell wall anchoring of SasK_{FLAG} requires cleavage of the N-terminal signal peptide of its P1 precursor and of the C-terminal sorting signal of the P2 precursor to generate mature anchored protein.

FIG. 4. The sorting signal of SasK is sufficient to anchor Seb to the cell wall envelope. Staphylococcal enterotoxin B (Seb) is a secreted protein. Fusion of the protein A sorting signal to the C-terminus of Seb causes cell wall anchoring of the hybrid protein as demonstrated by the cleavage of pulse-labeled P1 and P2 precursor to generate the mature anchored species. Cell wall anchoring of the Seb-Spa hybrid requires functional srtA but not srtB. Cell wall anchoring of Seb-SasK hybrids occurs under conditions when SrtB is expressed or over-expressed. Moreover, cell wall sorting of Seb-SasK does not require srtA.

FIG. 5. Purified SrtB cleaves NPQTN peptides in vitro. SrtA_{ΔN} and SrtB_{ΔN}, recombinant sortases with a six histidine tag replacing the N-terminal membrane anchor, were purified from E. coli extracts using affinity chromatography and were incubated with peptide substrate containing the NPQTN sequence motif. Peptides are modified with an N-terminal EDANS fluorophore and a C-terminal DABCYL fluorescence quencher and peptide cleavage was monitored as an increase in fluorescence over time. SrtB-mediated cleavage of NPQTN peptide was inhibited by MTSET, a methyl-methane thiosulfonate that forms disulfide with sulfhydryl residues. The addition of the strong reducing agent DTT greatly stimulated SrtB-mediated cleavage even in the presence of MTSET.

FIG. 6. Surface proteins and the pathogenesis of S. aureus infections. S. aureus Newman (human clinical isolate) and the isogenic mutants lacking SKM12 (srtA), SKM7 (srtB) or SKM14 (srtA/srtB) were injected into the tail vein of experimental mice as indicated. Five or nine days after infection, animals were sacrificed, kidneys excised, homogenized and plated. Symbols indicate colony forming units (cfu). The dashed line represents the limit of detection of staphylococci in renal tissues.

FIG. 7. Comparison of sortase A and sortase B sequences from S. aureus, L. monocytogenes and B. anthracis. Amino acid sequences were aligned with the BestFit program and are printed in a manner that reveals the relationship of sortase A and sortase B enzyme. Both A and B enzymes contain common sequence elements that are highlighted in light blue. Sortase A and sortase B family members are distinct due to the presence of unique sequence blocks (highlighted) that are present only in family members.

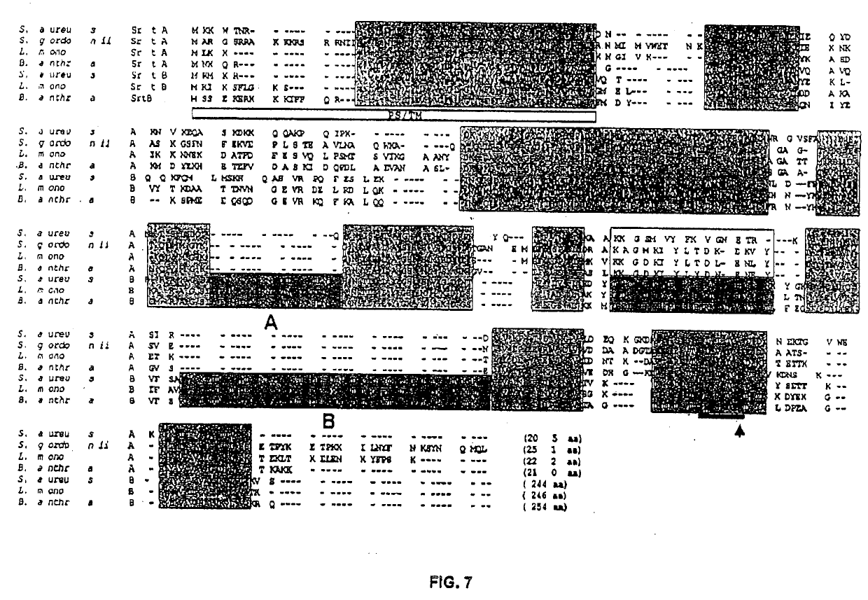

### DESCRIPTION OF ILLUSTRATIVE EMBODIMENTS

The present invention takes advantage of the observation of a sorting signal recognized by a sortase-transamidase from a Gram-positive bacterium. This sorting signal has the motif NPQ/KTN/G (asparagine, proline, glutamine or lysine, threonine, asparagine or glycine), which is cleaved by sortase B (SrtB). The present invention also concerns compositions and methods that can be used for preventative and therapeutic purposes, as well as screening and research purposes.

The invention concerns sortase-transamidase enzymes from a Gram-positive bacterium, including the enzymes identified as sortase A (srt A) and sortase B (srt B). Such enzymes may be substantially purified from a cell extract or lysate, which may be obtained from bacteria cells expressing native sortases or from cells expressing a recombinant sortase. As used herein, the term “substantially purified” means having a specific activity of at least tenfold greater than the sortase-transamidase activity present in a crude extract, lysate, or other state from which proteins have not been removed and also in substantial isolation from proteins found in association with sortase-transamidase in the cell.

### I. GRAM-POSITIVE BACTERIA

Bacteria have been classified into two groups: Gram-negative and Gram-positive. Gram-positive bacteria retain the crystal violet stain in the presence of alcohol or acetone. They have as part of their cell wall structure peptidoglycan as well as polysaccharides and/or teichoic acids. The peptidoglycans—sometimes also called murein—are heteropolymers of glycan strands, which are cross-linked through short peptides. Gram-positive bacteria include the following genera: Actinomyces, Bacillus, Bifidobacterium, Cellulomonas, Clostridium, Corynebacteriumk, Micrococcus, Mycobacterium, Nocardia, Staphylococcus, Streptococcus and Streptomyces.

A. Functionality of Cws in Cell Wall Anchoring

Screens for the functionality of Cws (cell wall sorting sequences) in anchoring fusion proteins in the cell wall have shown that five of ten Cws were functional in staphylococci. Ten surface protein Cws from various Gram-positive bacteria were fused to the C-terminus of staphylococcal enterotoxin B (Seb) or to the C-terminus of truncated protein A, which lacked its own Cws. (Schneewind et al., 1993). Without any further alteration, five of the ten were functional. Mutation of the remaining five allowed them to gain functionality. These altered, functional Cws displayed changes in the residue spacing between the LPXTG motif and the positively charged tail (Schneewind et al., 1993). Arginines at residue positions 31 and 32 downstream of the LPXTG motif act as the appropriate signal to retain the polypeptide within the secretory pathway. Changing the arginines to lysine retained the signalling function, but not if the change was to histidine. Therefore, a positive charge signals retention in the pathway. (Schneewind et al., 1993).

An initial BLAST search with the protein A Cws sequence followed by searches with other Cws so identified detected 19 surface protein genes, all of which appear to be exported by an N-terminal signal peptide. Nine of these encode well-characterized surface proteins: Spa, the bivrionectin-binding proteins FnbA and FnbB, the fibrinogen-binding clumping factors ClfA, ClfB, and SdrC, SdrD, SdrE and Pls, which contain serine and aspartate repeat regions upstream of the Cws (Uhlén et al., 1984; Flock et al, 1987; Jönsson et al., 1991; McDevitt et al., 1994; Josefsson et al., 1998a; Ní Eidhin et al., 1998). Ten of the genes found encode unknown surface proteins, identified here as S. aureus surface proteins, or sas. The S. aureus collagen adhesin (Cna), which has a Cws adhesion for bone tissue (Patti et al., 1992), is not in the four S. aureous genome sequences. Cna is found in isolates from bone and connective tissue infections by staphylococcus (Patti et al., 1994).

Each of these proteins has the Cws located at the C-terminal end. The LPXTG motif is also present in all, although the exact sequence varies. The residue at X may be acidic (glutamate or aspartate), neutral (alanine, glutamine, or asparagine), or basic (lysinel). In a single Cws sequence, the threonine (T) is replaced by alanine.

B. The Anchor Structure

The principles of how surface proteins are anchored are generally conserved. (Navarre and Schneewind, 1999). The surface proteins of S. aureus are linked through an amind bond between the carboxyl-group of threonine and the amino-group of the pentaglycine cell wall cross-bridge. Thus, the cell wall anchor may be released by treatment with N-acetylmuramidase, N-acetylglucosaminidase, N-acetylmuramyl-L-Ala amidase, D-Ala-Gly endopeptidase (Phi-11 enzyme) and lysostaphin. (Schneewind et al., 1995; Ton-That et al., 1997; Navarre et al., 1998 and 1999). Similarly, the C-terminal threonine of surface protein anchors in Listeria monocytogenes is linked through an amide bond to the side-chain amino-group of m-diaminopimelic acid within the cell wall peptides. (Dhar et al., 2000). Despite the variation in peptidoglycan structure in gram-positive bacteria, (Schleifer and Kandler, 1972) the means by which surface proteins are anchored is conserved.

C. Sortase-Transamidase Enzymes (SrtA and SrtB)

Sortase-transamidases are believed to occur in all Gram-positive bacteria. In particular, the enzyme exists in Mycobacterium, Nocardia, Actinomyces, Staphylococcus, Streptococcus, Listeria, Enterococcus, Bacillus, and Pneumococcus. Specifically, the enzyme exists in the following species: Staphylococcus aureus, S. sobrinus, Enterococcus faecalis, Streptococcus pyogenes, Bacillus subtilis, Streptococcus pneumoniae, and Listeria monocytogenes. Sorting signals for sortase-transamidase include, but are not limited to, those identified in TABLE 1.

TABLE 1

| | |
|---|---|
| NPQTN | SEQ ID NO. 5 |
| LPX₃X₄G | SEQ ID NO. 6 |

S. aureus

| | | | |
|---|---|---|---|
| LPETG | EENPFIGTTVFGGLSLALGAALLAG | RRREL | SEQ ID NO. 7 |
| LPETG | GEESTNKGMLFGGLFSILGLALL | RRNKKNHKA | SEQ ID NO. 8 |
| LPETG | GEESTNNGMLFGGLFSILGLALL | RRNKKNHKA | SEQ ID NO. 9 |
| LPDTG | SEDEANTSLIWGLLASIGSLLLF | RRKKENKDKK | SEQ ID NO. 10 |
| LPETG | DKSENTNATLFGAMMALLGSLLLF | RKRKQDHKEKA | SEQ ID NO. 11 |
| LPETG | SENNNSNNGTLFGGLFAALGSLLSFG | RRKKQNK | SEQ ID NO. 12 |
| LPETG | NENSGSNNATLFGGLFAALGSLLLFG | RRKKQNK | SEQ ID NO. 13 |
| LPETG | SENNGSNNATLFGGLFAALGSLLLFG | RRKKQNK | SEQ ID NO. 14 |
| LPDTG | NDAQNNGTLFGSLFAALGGLFLVG | RRRKNKNNEEK | SEQ ID NO. 15 |
| LPDTG | DSIKQNGLLGGVMTLLVGLGLM | KRKKKKDENDQDDSQA | SEQ ID NO. 16 |
| LPDTG | MSHNDDLPYAELALGAGMAFLI | RRFTKKDQQTEE | SEQ ID NO. 17 |
| LPNTG | SEGMDLPLKEFALITGAALLA | RRRTKN | SEQ ID NO. 18 |
| LPAAG | ESMTSSILTASIAALLLVSGLFLAF | RRRSTNK | SEQ ID NO. 19 |
| LPKTG | LTSVDNFISTVAFATLALLGSLSLLLF | KRKESK | SEQ ID NO. 20 |
| LPKAG | ETIKEHWLPISVIVGAMGVLMIWLS | RRNKLKNKA | SEQ ID NO. 21 |
| LPKTG | LESTQKGLIFSSIIGIAGLMLLA | RRRKN | SEQ ID NO. 22 |
| LPKTG | TNQSSSPEAMFVLLAGIGLIATV | RRRK | SEQ ID NO. 23 |
| LPKTG | ETTSSQSWWGLYALLGMLALFIP | KFRKESK | SEQ ID NO. 24 |
| LPQTG | EESNKDMTLPLMALLALSSIVAFVLP | RKRKN | SEQ ID NO. 25 |
| LPKTG | MKIITSWITWVFIGILGLYLIL | RKRFNS | SEQ ID NO. 26 |
| NPQTN | AGTPAYIYTIPVASLALLIAITLFV | RKKSKGNVE | SEQ ID NO. 33 |

S. pyogenes

| | | | |
|---|---|---|---|
| LPLAG | EVKSLLGILSIVLLGLLVLLYV | KKLKSRL | SEQ ID NO. 27 |
| LPATG | EKQHNMFFWMVTSCSLISSVFVISL | KTKKRLSSC | SEQ ID NO. 28 |
| LPSTG | EMVSYVSALGIVLVATITLYSIY | KKLKTSK | SEQ ID NO. 29 |
| QVPTG | VVGTLAPFAVLSIVAIGGVIYIT | KRKKA | SEQ ID NO. 30 |
| VPPTG | LTTDGAIYLWLLLLVPFGLLVWLFG | RKGLKND | SEQ ID NO. 31 |
| EVPTG | VAMTVAPYIALGIVAVGGALYFV | KKKNA | SEQ ID NO. 32 |

Sortase A from Staphylococcus aureus has a molecular weight of about 23,539 daltons. It catalyzes a reaction that covalently crosslinks the carboxyl-terminus of a protein having a sorting signal to the peptidoglycan of the Gram-positive bacterium. The sorting signal for Srt A has: (1) a motif of LPX₃X₄G therein; (2) a substantially hydrophobic domain of at least 31 amino acids carboxyl to the motif; and (3) a charged tail region with at least two positively charged residues carboxyl to the substantially hydrophobic domain, at least one of the two positively charged residues being arginine, the two positively charged residues being located at residues 31-33 from the motif. In this sorting signal, X₃ can be any of the twenty naturally-occurring L-amino acids. Xa can be alanine, serine, or threonine. Preferably, X₄ is threonine. Sortase A is an enzyme of 206 amino acids encoded by the gene srtA. The gene was identified through a screen for mutants incapable of cleaving the protein A Cws (Mazmanian et al., 1999). Sortase A has a putative N-terminal membrane-spanning domain. The C-terminus contains a catalytic domain that may be translocated across the cytoplasmic membrane.

The amino acid sequence of SrtA from S. aureus is provided below:

M-K-K-W-T-N-R-L-M-T-1-A-G-V-V-L-1-L-V-A-A-Y-L-F-A-K-P-H-I-D-N-Y-L-H-D-K-D-K-D-E-K-I-E-Q-Y-D-K-N-V-K-E-Q-A-S-K-D-K-K-Q-Q-A-K-P-Q-I-P-K-D-K-S-K-V-A-G-Y-I-E-I-P-D-A-D-I-K-E-P-V-Y-P-G-P-A-T-P-E-Q-L-N-R-G-V-S-F-A-EE-N-E-S-L-D-D-Q-N-1-S-I-A-G-H-T-F-I-D-R-P-N-Y-Q-F-T-N-L-K-A-A-K-K-G-S-M-V-YF-K-V-G-N-E-T-R-K-Y-K-M-T-S-I-R-D-V-K-P-T-D-V-G-V-L-D-E-Q-K-G-K-D-K-Q-L-T-L-I-T-C-D-D-Y-N-E-K-T-G-V-W-E-K-R-K-I-F-V-A-T-E-V-K (SEQ ID NO: 2). The DNA encoding sequence is provided as SEQ ID NO:1.

The sortase-transamidase is a cysteine protease. The Srt A enzyme first cleaves a polypeptide having a sorting signal within the LPX3X4G motif. Cleavage occurs after residue X₄, normally a threonine; as indicated above, this residue can also be a serine or alanine residue. This residue forms a covalent intermediate with the sortase. The next step is the transamidation reaction that transfers the cleaved carboxyl terminus of the protein to be sorted to the —NH₂ of the pentaglycine crossbridge within the peptidoglycan precursor. The peptidoglycan precursor is then incorporated into the cell wall by a transglycosylase reaction with the release of undecaprenyl phosphate. The mature anchored polypeptide chains are thus linked to the pentaglycine cross bridge in the cell wall which is tethered to the s-amino side chain of an unsubstituted cell wall tetrapeptide. A carboxypeptidase may cleave a D-Ala-D-Ala bond of the pentapeptide structure to yield the final branched anchor peptide in the staphylococcal cell wall.

The Srt A sorting signal has: (1) a motif of LPX;X4G therein; (2) a substantially hydrophobic domain of at least 31 amino acids carboxyl to the motif; and (3) a charged tail region.

In the Motif, X₃ can be any of the 20 naturally-occurring L-amino acids. X₄ can be any of threonine, serine, or alanine. Preferably, X₄ is threonine (Schneewind et al., 1993). Preferably, the substantially hydrophobic domain carboxyl to the motif includes no more than about 7 charged residues or residues with polar side chains. For the purposes of this specification, these residues include the following: aspartic acid, glutamic acid, lysine, and arginine as charged residues, and serine, threonine, glutamine, and asparagine as polar but uncharged residues. Preferably, the sequence includes no more than three charged residues. Representative sequences suitable for sorting signals for use with the sortase-transamidase of the present invention include, but are not limited to the following: SEQ ID NO:5-33, inclusive. Other hydrophobic domains can be used as part of the sorting signal.

The third portion of the sorting signal is a charged tail region with at least two positively charged residues carboxyl to the substantially hydrophobic domain. At least one of the two positively charged residues is arginine. The charged tail can also contain other charged amino acids, such as lysine. Preferably, the charged tail region includes two or more arginine residues. The two positively charged residues are located at residues 31-33 from the motif. Preferably, the two arginine residues are either in succession or are separated by no more than one intervening amino acid. Preferably, the charged tail is at least five amino acids long, although four is possible.

Although sortase knock-out mutants were not deleterious to growth on laboratory media, the lack of sortase activity results in accumulation of precursor protein in the cytoplasm, membrane, and cell wall fractions. (Mazmanian et al., 2000). Further, the Cws of fibronectin-binding proteins (FnbA and FnbB), and that of the clumping factor ClfA are not cleaved in these mutants. Knock out mutations of srtA may therefore casue a defect in the anchoring of a number of surface proteins (Mazmanian et al., 2000). These mutants also do not present Ig, fibronectin and fibrinogen adhesins on the staphylococcal surface, which also indicates that they produce a general defect in the sorting of proteins to the cell wall (Mazmanian et al., 2000).

Interestingly, a second sortase, SrtB has been identified in S. aureus through a BLAST search using the srtA gene sequence (Pallen, et al., 2001). In contrast to the results for knock-out mutants of srtA, replaceing the srtB gene of S. aureus Newman with the ermC marker does not disrupt the cell wall anchoring of protein A, FnbA, FnbB, or ClfA. The anchoring of other cell surface proteins may be affected, however. In any case, though related, the functions of SrtA and SrtB are not redundant. The amino acid sequence for Srt B from Stapholococcus aureus is provided in SEQ ID NO:4. The nucleic acid sequence encoding Srt B is provided in SEQ ID NO:3. The sorting signal that is cleaved by Srt B is NPQTN.

The role of sortase in S. aureus infection was assessed in the murine renal abscess model. Knock out mutants of srtA produced 100 to 1000 times fewer viable, recoverable staphylococci when compared to the human clinical isolate, S. aureus Newman. Intraperitoneal injection in a murine model showed an LD50 reduced by 1.5 on a log scale when compared to S. aureus Newman (Mazmanian et al, 2000).

Homologs of S. aureus srtA are present in all Gram-positive bacteria for which whole genomic sequence is available. These include Actinomyces naeslundii, Bacillus anthracis, Bacillus subtilis, Clostridium acetabutylicum, Corynebacterium diptheriae, Enterococcus faecalis, Listeria monocytogenes, Streptococcus mutans, Streptococcus pheumoniae and Streptococcus pyogenes (Mazmanian et al., 1999). The number of sortase genes present may be as high as three or more (Pallen et al., 2001). Oddly, Methanobacterium thermoautotrophicum contains two sortase genes, despite its ability to synthesize pseudopeptidoglycan, (N-acetylglucosamine-(Beta 1-3)-N-acetyltalosaminurate polymer) instead of peptidoglycan (Kandler and König, 1998; Pallen et al, 2001) and despite that no genes encoding a surface protein with a C-terminal Cws have been identified to date in Methanobacterium (Pallen et al, 2001). Perhaps sortase links proteins to the amino-group of lysine, since the side-chain of this residue is engaged in cross-linkage with gamma-glutamyl of neighboring peptide subunits (Kandler and Konig, 1998).

Despite this oddity, the role of SrtA in anchoring surface proteins to the cell wall of Gram-positive bacteria appears to be conserved. Knock-out mutations in other species demonstrate the role of SrtA. In Streptococcus gordonii, such knock-out mutants of srtA disrupts the anchoring and display of cell surface proteins, resulting in the loss of the adhesive properties of wild type bacteria (Bolken et al, 2001). In Actinomyces species, the fimbriae are composed of Cws-bearing subunit proteins and knock-out mutations of the srtA homolog abolishes processing at the C-terminal LPXTG motif (Yeung et al, 1998). Although, the exact nature of the linkage of the fimbral proteins and the cell wall is not yet clear (Yeung et al, 1998; Pallen et al., 2001).

### II. SORTASE-TRANSAMIDASE AS A TARGET FOR ANTIBIOTIC ACTION

A. A Site for Antibiotic Action

The reaction carried out by the sortase-transamidase of the present invention presents a possible target for a new class of antibiotics to combat medically relevant infections caused by numerous Gram-positive organisms. Because this is a novel site of antibiotic action, these antibiotics have the advantage that resistance by the bacterium has not had a chance to develop.

Such antibiotics can include compounds with structures that mimic the cleavage site, such as compounds with a structure similar to methyl methanethiosulfonate or, more generally, alkyl methanethiosulfonates. The sortase-transamidase of the present invention is believed to be a cysteine protease. Other antibiotics that may inhibit the activity of the sortase-transamidase in the present invention include inhibitors that would be specific for cysteine-modification in a β-lactam framework. These inhibitors can, but need not necessarily, have active moieties that would form mixed disulfides with the cysteine sulfhydryl. These active moieties could be derivatives of methanethiosulfonate, such as methanethiosulfonate ethylammonium, methanethiosulfonate ethyltrimethylammonium, or methanethiosulfonate ethylsulfonate (J. A. Javitch et al., 1995; Akabas et al., 1995). Similar reagents, such as alkyl alkanethiosulfonates, i.e., methyl methanethiosulfonate, or alkoxycarbonylalkyl disulfides, have been described (Smith et al., 1975; Valentine et al., 1981). Other useful inhibitors involve derivatives of 2-trifluoroacetylaminobenzene sulfonyl fluoride (Powers, “Proteolytic Enzymes and Their Active-Site-Specific Inhibitors: Role in the Treatment of Disease,” in Modification of Proteins), in a (β-lactam framework, peptidyl aldehydes and nitriles (Dufour et al. 1995; Westerik et al., 1972), peptidyl diazomethyl ketones (Björck et al., 1989), peptidyl phosphonamidates (Bartlett et al., 1983), phosphonate monoesters such as derivatives or analogues of m-carboxyphenyl phenylacetamidomethylphosphonate (Pratt, 1989), maleimides and their derivatives, including derivatives of such bifunctional maleimides as o-phenylenebismaleimide, p-phenylenebismaleimide, m-phenylenebismaleimide, 2,3-naphthalenebismaleimide, 1,5-naphthalenebismaleimide, and azophenylbismaleimide, as well as monofunctional maleimides and their derivatives (Moroney et al., 1982), peptidyl halomethyl ketones (chloromethyl or fluoromethyl ketones), peptidyl sulfonium salts, peptidyl acyloxymethyl ketones, derivatives and analogues of epoxides, such as E-64 (N-[N-(L-trans-carboxyoxiran-2-carbonyl)-L-leucylagmatine), E-64c (a derivative of E-64 in which the agmatine moiety is replaced by an isoamylamine moiety), E-64c ethyl ester, Ep-459 (an analogue of E-64 in which the agmatine moiety is replaced by a 1,4-diaminopropyl moiety), Ep-479 (an analogue of E-64 in which the agmatine moiety is replaced by a 1,7-diheptylamino moiety), Ep-460 (a derivative of Ep-459 in which the terminal amino group is substituted with a Z (benzyloxycarbonyl) group), Ep-174 (a derivative of E-64 in which the agmatine moiety is removed, so that the molecule has a free carboxyl residue from the leucine moiety), Ep-475 (an analogue of E-64 in which the agmatine moiety is replaced with a NH₂—(CH₂)₂—CH—(CH₃)₂ moiety), or Ep-420 (a derivative of E-64 in which the hydroxyl group is benzoylated, forming an ester, and the leucylagmatine moiety is replaced with isoleucyl-O-methyltyrosine), or peptidyl O-acyl hydroxamates (E Shaw, “Cysteinyl Proteases and Their Selective Inactivation), pp 271-347). Other inhibitors are known in the art. Modification of other residues may also result in inhibition of the enzyme.

B. Screening Methods

Another aspect of the present invention is a method for screening a compound for anti-sortase-transamidase activity. This is an important aspect of the present invention, because it provides a method for screening for compounds that disrupt the sorting process and thus have potential antibiotic activity against Gram-positive bacteria.

In general, this method comprises the steps of: (1) providing an active fraction of sortase-transamidase enzyme; (2) performing an assay for sortase-transamidase activity in the presence and in the absence of the compound being screened; and (3) comparing the activity of the sortase-transamidase enzyme in the presence and in the absence of the compound.

The active fraction of sortase-transamidase enzyme can be a substantially purified sortase-transamidase enzyme preparation according to the present invention, but can be a less purified preparation, such as a partially purified particulate preparation as described below.

The enzymatic activity can be measured by the cleavage of a suitable substrate, such as the construct having the Staphylococcal Enterotoxin B (SEB) gene fused to the cell wall sorting signal of Staphylococcal Protein A (SPA). The cleavage can be determined by monitoring the molecular weight of the products by sodium dodecyl sulfatepolyacrylamide gel electrophoresis or by other methods.

One particularly assay for sortase-transamidase activity is the following: Staphylococcal soluble RNA (sRNA) is prepared from S. aureus by a modification of the technique of Zubay (Zubay, 1962). An overnight culture of S. aureus is diluted 1:10 in TSB and incubated at 37° C. for 3 hr. The cells are harvested by centrifugation at 6000 rpm for 15 min. For every gram of wet cell pellets, 2 ml of 0.01 M magnesium acetate, 0.001 M Tris, pH 7.5 is used to suspend the pellets. The cell pellets are beaten by glass bead beater for 45 minutes in 5 minute intervals. The suspension is centrifuged twice at 2500 rpm for minutes to remove the glass beads, then 0.5 ml phenol is added to the suspension. The suspension is vigorously shaken for 90 min at 4° C., and then centrifuged at 18,000×g for 15 min. The nucleic acids in the top layer are precipitated by addition of 0.1 volume of 20% potassium acetate and 2 volumes of ethanol, then stored at 4° C. for at least 36 hrs. The precipitate is obtained by centrifugation at 5,000×g for 5 min. Cold NaCl (1 ml) is added to the precipitate and stirred at 4° C. for 1 hr. The suspension is centrifuged at 15,000×g for 30 min. The sediments are washed with 0.5 ml of cold 1 M NaCl. The supernatants are combined and 2 volumes of ethanol is added to precipitate the tRNA. The precipitate is suspended in 0.1 ml of 0.2 M glycine, pH 10.3 and incubated for 3 hr at 37° C. This suspension is then made 0.4 M in NaCl and the RNA is precipitated by addition of 2 volumes of ethanol. The precipitate is dissolved in 0.7 ml of 0.3 M sodium acetate, pH 7.0. To this is slowly added 0.5 volume of isopropyl alcohol, with stirring. The precipitate is removed by centrifugation at 8,000×g for 5 min. This precipitate is redissolved in 0.35 ml of 0.3 M sodium acetate, pH 7.0. To this is added 0.5 volume of isopropyl alcohol, using the same procedure as above. The precipitate is also removed by centrifugation. The combined supernatants from the two centrifugations are treated further with 0.37 ml of isopropyl alcohol. The resulting precipitate is dissolved in 75 μl of water and dialyzed against water overnight at 4° C. This sRNA is used in the sortase-transamidase assay.

Particulate sortase-transamidase enzyme is prepared for use in the assay by a modification of the procedure of Chatterjee & Park (Chatterjee et al., 1964). An overnight culture of S. aureus OS2 is diluted 1:50 in TSB and incubated at 37° C. for 3 hr. Cells are harvested by centrifugation at 6000 rpm for 15 minutes, and washed twice with ice-cold water. The cells are disrupted by shaking 7 ml of 1 3% suspension of cells in 0.05 M Tris-HCl buffer, pH 7.5, 0.1 mM MgCl₂, and 1 mM 2-mercaptoethanol with an equal volume of glass beads for 10-15 min in a beater. The glass beads are removed by centrifugation at 2000 rpm for 5 min. The crude extract is then centrifuged at 15,000×g for min. The supernatant is centrifuged again at 100,000×g for 30 min. The light yellow translucent pellet is resuspended in 2 to 4 ml of 0.02 M Tris-HCl buffer, pH 7.5, containing 0.1 mM MgCl₂ and 1 mM 2-mercaptoethanol. This suspension represents the crude particulate enzyme and is used in the reaction mixture below.

The supernatant from centrifugation at 100,000×g is passed through gel filtration using a Sephadex® G-25 agarose column (Pharmacia) to remove endogenous substrates. This supernatant is also used in the reaction mixture.

The complete reaction mixture contains in a final volume of 30 μl (M. Matsuhashi et al., 1965): 3 μmol of Tris-HCl, pH 7.8; 0.1 μmol of MgCl₂; 1.3 μmol of KCl; 2.7 mmol of [³H] glycine (200 μCi/μmol); 2 nmol of UDP-M-pentapeptide; 5 nmol of UDP-N-acetylglucosamine; 0.2 μmol of ATP; 0.05 μmol of potassium phosPhoenolpyruvate; 2.05 μg of chloramphenicol; 5 μg of pyruvate kinase; 0.025 μmol of 2-mercaptoethanol; 50 μg of staphylococcal sRNA prepared as above; 4 μg (as protein) of supernatant as prepared above; 271 μg of particulate enzyme prepared as above: and 8 nmol of a synthesized soluble peptide (HHHHHHAQALEPTGEENPF) as a substrate.

The mixture is incubated at 20° C. for 60 min. The mixture is then heated at 100° C. for 1 min. The mixture is diluted to 1 ml and precipitated with 50 μl nickel resin, and washed with wash buffer (1% Triton X-100, 0.1% sodium dodecyl sulfate, 50 mM Tris, pH 7.5). The nickel resin beads are counted in a scintillation counter to determine ³H bound to the beads.

The effectiveness of the compound being screened to inhibit the activity of the sortase-transamidase enzyme can be determined by adding it to the assay mixture in a predetermined concentration and determining the resulting degree of inhibition of enzyme activity that results. Typically, a dose-response curve is generated using a range of concentrations of the compound being screened.

The particulate enzyme preparation of sortase-transamidase employed in this protocol can be replaced with any other sortase-transamidase preparation, purified or crude, staphylococcal, recombinant, or from any other source from any other Gram-positive bacterium as described above.

The soluble peptide is captured in this embodiment by its affinity for nickel resin as a result of the six histidine residues. More than six histidine residues can be used in the peptide. As an alternative, the soluble peptide can be captured by an affinity resulting from other interactions, such as streptavidin-biotin, glutathione S-transferase-glutathione, maltose binding protein-amylose, and the like, by replacing the six histidine residues with the amino acid sequence that constitutes the binding site in the peptide and employing the appropriate solid phase affinity resin containing the binding partner. Suitable peptides can be prepared by solid phase peptide synthesis using techniques well known in the art, such as those described in M. Bodanszky, 1993). For example, if the glutathione S-transferase-glutathione interaction is used, the active site of glutathione S-transferase (Smith and Johnson, 1988) can be substituted for the six histidine residues, and glutathione can be bound to the solid support.

Alternatively, the soluble peptide can be released from the sortase by hydroxylaminolysis and then quantitated or monitored. The strong nucleophile hydroxylamine attacks thioester to form hydroxamate with carboxyl, thereby regenerating the enzyme sulfhydryl. Hydroxylaminolysis can be carried out in 50 mM Tris-HCl, pH 7.0 with a concentration of 0.1 M hydroxylamine for 60 min. The released peptide, for example, can be quantitated by mass spectroscopy or other methods.

### IV. USE OF SORTASE-TRANSAMIDASE FOR PROTEIN AND PEPTIDE DISPLAY

A. Methods for Protein and Peptide Display

The sortase-transamidase enzyme of the present invention can also be used in a method of displaying a polypeptide on the surface of a gram-positive bacterium. In general, a first embodiment of this method comprises the steps of: (1) expressing a polypeptide having a sorting signal at its carboxyl-terminal end as described above; (2) forming a reaction mixture including: (i) the expressed polypeptide; (ii) a substantially purified sortase-transamidase enzyme; and (iii) a Gram-positive bacterium having a peptidoglycan to which the sortase-transamidase can link the polypeptide; and (3) allowing the sortase-transamidase to catalyze a reaction that cleaves the polypeptide within a LPX₃X₄G motif or a NPQ/KTN/G motif of the sorting signal and covalently cross-links the amino-terminal portion of the cleaved polypeptide to the peptidoglycan to display the polypeptide on the surface of the Gram-positive bacterium.

In this method, the polypeptide having the sorting signal at its carboxy terminal end need not be expressed in a Gram-positive bacterium; it can be expressed in another bacterial system such as Escherichia coli or Salmonella typhimuriurn, or in a eukaryotic expression system.

The other method for protein targeting and display relies on direct expression of the chimeric protein in a Gram-positive bacterium and the action of the sortase-transamidase on the expressed protein. In general, such a method comprises the steps of: (1) cloning a nucleic acid segment encoding a chimeric protein into a Gram-positive bacterium to generate a cloned chimeric protein including therein a carboxyl-terminal sorting signal as described above, the chimeric protein including the polypeptide to be displayed; (2) growing the bacterium into which the nucleic acid segment has been cloned to express the cloned chimeric protein to generate a chimeric protein including therein a carboxyl-terminal sorting signal; and (3) covalent binding of the chimeric protein to the cell wall by the enzymatic action of the sortase-transamidase involving cleavage of the chimeric protein within the LPX₃X₄G motif or NPQ/KTN/G motif so that the protein is displayed on the surface of the gram-positive bacterium in such a way that the protein is accessible to a ligand.

Typically, the Gram-positive bacterium is a species of Staphylococcus. A particularly preferred species of Staphylococcus is Staphylococcus aureus. However, other Gram-positive bacteria such as Streptococcus pyogenes, other Streptococcus species, and Gram-positive bacteria of other genera can also be used.

Cloning the nucleic acid segment encoding the chimeric protein into the Gram-positive bacterium is performed by standard methods. In general, such cloning involves: (1) isolation of a nucleic acid segment encoding the protein to be sorted and covalently linked to the cell wall; (2) joining the nucleic acid segment to the sorting signal; (3) cloning by insertion into a vector compatible with the Gram-positive bacterium in which expression is to take place; and (4) incorporation of the vector including the new chimeric nucleic acid segment into the bacterium.

Typically, the nucleic acid segment encoding the protein to be sorted is DNA; however, the use of RNA in certain cloning steps is within the scope of the present invention.

When dealing with genes from eukaryotic organisms, it is preferred to use cDNA, because the natural gene typically contains intervening sequences or introns that are not translated. Alternatively, if the amino acid sequence is known, a synthetic gene encoding the protein to be sorted can be constructed by standard solid-phase oligodeoxyribonucleotide synthesis methods, such as the phosphotriester or phosphite triester methods. The sequence of the synthetic gene is determined by the genetic code, by which each naturally occurring amino acid is specified by one or more codons. Additionally, if a portion of the protein sequence is known, but the gene or messenger RNA has not been isolated, the amino acid sequence can be used to construct a degenerate set of probes according to the known degeneracy of the genetic code. General aspects of cloning are described, for example, in Sambrook et al., 1989); in B. Perbal, 1988), in S. L. Berger & A. R. Kimmel, 1987), and in D. V. Goeddel, ed., 1991).

Once isolated, DNA encoding the protein to be sorted is then joined to the sorting signal. This is typically accomplished through ligation, such as using Escherichia coti or bacteriophage T4 ligase. Conditions for the use of these enzymes arc well known and are described, for example, in the above general references.

The ligation is done in such a way so that the protein to be sorted and the sorting signal are joined in a single contiguous reading frame so that a single protein is produced. This may, in some cases, involve addition or deletion of bases of the cloned DNA segment to maintain a single reading frame. This can be done by using standard techniques.

Cloning is typically performed by inserting the cloned DNA into a vector containing control elements to allow expression of the cloned DNA. The vector is then incorporated into the bacterium in which expression is to occur, using standard techniques of transformation or other techniques for introducing nucleic acids into bacteria.

One suitable cloning system for S. aureus places the cloned gene under the control of the B1aZRI regulon (P. Z. Wang et al., 1991). Vectors and other cloning techniques for use in Staphylococcus aureus are described in B. Nilsson & L. Abrahmsen, “Fusion to Staphylococcal Protein A.” in Gene Expression Technology, supra, p. 144-161.

If the chimeric protein is cloned under control of the B1aZR1 regulon, expression can be induced by the addition of the β-lactam antibiotic methicillin.

Another aspect of the present invention is a polypeptide displayed on the surface of a Gram-positive bacterium by covalent linkage of an amino-acid sequence of LPX₃X₄ derived from cleavage of an LPX₃X₄G motif, as described above. Alternatively a sorting signal may have the NPQ/KTN/G motif and be cleaved within that sequence.

Yet another aspect of the present invention is a covalent complex comprising: (1) the displayed polypeptide; and (2) an antigen or hapten covalently cross-linked to the polypeptide.

B. Screening Methods

These polypeptides associated with the cell surfaces of Gram-positive bacteria can be used in various ways for screening. For example, samples of expressed proteins from an expression library containing expressed proteins on the surfaces of the cells can be used to screen for clones that express a particular desired protein when a labeled antibody or other labeled specific binding partner for that protein is available. Screens may be implemented with any sortase-transamidase, including Srt A and Srt B; moreover, a combination of screens, wherein the same sortase-transamidase but from different organisms is screened and/or a different sortase-transamidase from the same organisms may be screened, or a combination thereof. Multiple screens may be employed using different sortase transamidases, either from the same organism or from different organism. The more sortase-transamidases are inhibited by a prticular candidate compound the more valuable it becomes as a possible broad-spectrum antibioitic.

These methods are based on the methods for protein targeting and display described above. A first embodiment of such a method comprises: (1) expressing a cloned polypeptide as a chimeric protein having a sorting signal at its carboxy-terminal end as described above; (2) forming a reaction mixture including: (i) the expressed chimeric protein; (ii) a substantially purified sortase-transamidase enzyme; and (iii) a Gram-positive bacterium having a peptidoglycan to which the sortase-transamidase can link the polypeptide through the sorting signal; (3) binding of the chimeric protein covalently to the cell wall by the enzymatic action of a sortase-transamidase expressed by the Gram-positive bacterium involving cleavage of the chimeric protein within the LPX₃X₄G motif or NPQ/KTN/G motif so that the polypeptide is displayed on the surface of the Gram-positive bacterium in such a way that the polypeptide is accessible to a ligand; and (4) reacting the displayed polypeptide with a labeled specific binding partner to screen the chimeric protein for reactivity with the labeled specific binding partner. The nucleic acid segment encoding the chimeric protein is formed by methods well known in the art and can include a spacer. In the last step, the cells are merely exposed to the labeled antibody or other labeled specific binding partner, unreacted antibodies removed as by a wash, and label associated with the cells detected by conventional techniques such as fluorescence, chemiluminescence, or autoradiography.

A second embodiment of this method employs expression in a Gram-positive bacterium that also produces a sortase-transamidase enzyme. This method comprises: (1) cloning a nucleic acid segment encoding a chimeric protein into a Gram-positive bacterium to generate a cloned chimeric protein including therein a carboxyl-terminal sorting signal as described above, the chimeric protein including the polypeptide whose expression is to be screened; (2) growing the bacterium into which the nucleic acid segment has been cloned to express the cloned chimeric protein to generate a chimeric protein including therein a carboxyl-terminal sorting signal; (3) binding the polypeptide covalently to the cell wall by the enzymatic action of a sortase-transamidase expressed by the Gram-positive bacterium involving cleavage of the chimeric protein within a LPX3X4G motif or NPQ/KTN/G motif so that the polypeptide is displayed on the surface of the Gram-positive bacterium in such a way that the polypeptide is accessible to a ligand; and (4) reacting the displayed polypeptide with a labeled specific binding partner to screen the chimeric protein for reactivity with the labeled specific binding partner.

The present invention further comprises methods for identifying modulators of a sortase-transamidase. Modulation of a sortase-transamidase involves altering or changing its transcription, translation expression (transcription+translation), post-translational modification, processing, turnover rate, location or translocation, secretion, specificity, activity and/or function of a sortase-transamidase may be altered. Thus, inhibiting, reducing, increasing, or enhancing any of the previous characteristics constitutes modulating the protein. One modulation that is desirable is a modulation of the cleavage activity. These assays may comprise random screening of large libraries of candidate substances; alternatively, the assays may be used to focus on particular classes of compounds selected with an eye towards structural attributes that are believed to make them more likely to modulate the activity of a sortase-transamidase.

By activity, it is meant that one may assay for a measurable effect on a candidate substance activity or sortase-transamidase inhibition by the candidate substance. To identify a sortase-transamidase modulator, one generally will determine the activity or level of inhibition of a sortase-transamidase in the presence and absence of the candidate substance, wherein a modulator is defined as any substance that alters these characteristics. For example, a method generally comprises: (a) providing a candidate modulator; (b) admixing the candidate modulator with an isolated compound or cell, or a suitable experimental animal; (c) measuring one or more characteristics of the compound, cell or animal in step (b); and (d) comparing the characteristic measured in step (c) with the characteristic of the compound, cell or animal in the absence of said candidate modulator, wherein a difference between the measured characteristics indicates that said candidate modulator is, indeed, a modulator of the compound, cell or animal.

Assays may be conducted in cell free systems, in isolated cells, or in organisms including transgenic animals. Alternatively, screening may be high-throughput. In some cases it may involve attaching one or more components to a nonreacting substance or implementing chip technology. It will, of course, be understood that all the screening methods of the present invention are useful in themselves notwithstanding the fact that effective candidates may not be found. The invention provides methods for screening for such candidates, not solely methods of finding them.

As used herein the term “candidate substance” refers to any molecule that may potentially modify sortase-transamidase activity. The candidate substance may inhibit or enhance sortase-transamidase activity. The candidate substance may be a protein or fragment thereof, a small molecule, or even a nucleic acid molecule. An example of pharmacological compounds will be compounds that are structurally related to sortase-transamidase, or a substrate of a sortase-transamidase. Using lead compounds to help develop improved compounds is know as “rational drug design” and includes not only comparisons with known inhibitors and activators, but predictions relating to the structure of target molecules. An “inhibitor” is a molecule which represses or prevents another molecule from engaging in a reaction. An “activator” is a molecule that increases the activity of an enzyme or a protein that increases the production of a gene product in DNA transcription.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are well known to those of skill in the art. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

An inhibitor according to the present invention may be one which exerts its inhibitory or activating effect upstream, downstream or directly on a sortase-transamidase. Regardless of the type of inhibitor or activator identified by the present screening methods, the effect of the inhibition or activator by such a compound results in alteration in sortase-transamidase enzymatic activity as compared to that observed in the absence of the added candidate substance.

C. In Vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

One example of a cell free assay is a binding assay. While not directly addressing function, the ability of a modulator to bind to a target molecule such as a dehydrogenase or 11-cis-retinal dehydrogenase in a specific fashion is strong evidence of a related biological effect. For example, binding of a molecule to a target may, in and of itself, be inhibitory, due to steric, allosteric or charge-charge interactions. The target may be either free in solution, fixed to a support, expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide is detected by various methods.

D. In Cyto Assays

The present invention also contemplates the screening of compounds for their ability to modulate the activity of sortase-transamidase in cells. Various cell lines can be utilized for such screening assays, including cells specifically engineered for this purpose. Any Gram-positive bacteria may be employed for these assays as well.

Depending on the assay, culture may be required. The cell is examined using any of a number of different physiologic assays. Alternatively, molecular analysis may be performed, for example, looking at protein expression, mRNA expression (including differential display of whole cell or polyA RNA) and others.

E. In Vivo Assays

In vivo assays involve the use of various animal models, including transgenic animals that have been engineered to have specific defects, or carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal model derived from any of these species.

In the current invention, the preferred animal model for screening for modulation in the function of a sortase-transamidase is a murine renal abscess model (Albus et al., 1991).

In such assays, one or more candidate substances are administered to an animal that has been infected or will be infected with a Gram-positive bacteria, and the ability of the candidate substance(s) to alter one or more characteristics, as compared to a similar animal not treated with the candidate substance(s), identifies a modulator. The characteristics may be any of those discussed above with regard to the function of a particular compound (e.g., enzyme, receptor, hormone) or cell (e.g., growth, tumorigenicity, survival), or instead a broader indication such as behavior, anemia, immune response, etc. In this particular case, abscess formation, pathogenicity or virulence can be evaluated.

The present invention provides methods of screening for a candidate substance that changes the activity of one or more sortase-transamidases such as SrtA or SrtB or both. In these embodiments, the present invention is directed to a method for determining the ability of a candidate substance to change the activity of a sortase-transamidase, generally including the steps of: administering a candidate substance to the animal; and determining the ability of the candidate substance to reduce one or more characteristics of a sortase-transamidase.

Treatment of these animals with test compounds will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

Determining the effectiveness of a compound in vivo may involve a variety of different criteria. Also, measuring toxicity and dose response can be performed in animals in a more meaningful fashion than in in vitro or in cyto assays.

### V. USE OF SORTED MOLECULES FOR DIAGNOSIS AND TREATMENT OF BACTERIAL INFECTIONS

Sorted molecules can also be used for the diagnosis and treatment of bacterial infections caused by Gram-positive bacteria. Antibiotic molecules or fluorescent or any other diagnostic molecules can be chemically linked to a sorted peptide segment, which may include a spacer as described above, and then can be injected into animals or humans. These molecules are then sorted by the sortase-transamidase so that they are covalently linked to the cell wall of the bacteria.

In general, these methods comprise: (1) conjugating an antibiotic or a detection reagent to a protein including therein a carboxyl-terminal sorting signal to produce a conjugate; and (2) introducing the conjugate to an organism infected with a Gram-positive bacterium in order to cause the conjugate to be sorted and covalently cross-linked to the cell walls of the bacterium in order to treat or diagnose the infection.

The antibiotic used can be, but is not limited to, a penicillin, ampicillin, vancomycin, gentamicin, streptomycin, a cephalosporin, amikacin, kanamycin, neomycin, paromomycin, tobramycin, ciprofloxacin, clindamycin, rifampin, chloramphenicol, or norfloxacin, or a derivative of these antibiotics.

The detection reagent is typically an antibody or other specific binding partner labeled with a detectable label, such as a radiolabel. Such methods are well known in the art and need not be described further here.

Accordingly, another aspect of the present invention is a conjugate comprising an antibiotic or a detection reagent covalently conjugated to a protein including therein a carboxyl-terminal sorting signal as described above to produce a conjugate.

Yet another aspect of the present invention is a composition comprising the conjugate and a pharmaceutically acceptable carrier. In this context, the conjugates can be administered using conventional modes of administration, including, but not limited to, intravenous, intraperitoneal, oral, or intralymphatic. Other routes of administration can alternatively be used. Oral or intraperitoneal administration is generally preferred. The composition can be administered in a variety of dosage forms, which include, but are not limited to, liquid solutions or suspensions, tablets, pills, powders, suppositories, polymeric microcapsules or microvesicles, liposomes, and injectable or infusible solutions. The preferred form depends on the mode of administration and the quantity administered.

The compositions for administration preferably also include conventional pharmaceutically acceptable carriers and adjuvants known in the art such as human serum albumin, ion exchangers, alumina, lecithin, buffered substances such as phosphate, glycine, sorbic acid, potassium sorbate, and salts or electrolytes such as protamine sulfate. The most effective mode of administration and dosage regimen for the conjugates as used in the methods in the present invention depend on the severity and course of the disease, the patient's health, the response to treatment, the particular strain of bacteria infecting the patient, other drugs being administered and the development of resistance to them, the accessibility of the site of infection to blood flow, pharmacokinetic considerations such as the condition of the patient's liver and/or kidneys that can affect the metabolism and/or excretion of the administered conjugates, and the judgment of the treating physician. According, the dosages should be titrated to the individual patient.

### VI. USE OF SORTED POLYPEPTIDES FOR PRODUCTION OF VACCINES

Additionally, the sorted polypeptides covalently crosslinked to the cell walls of Gram-positive bacteria according to the present invention have a number of uses. One use is use in the production of vaccines that can be used to generate immunity against infectious diseases affecting mammals, including both human and non-human mammals, such as cattle, sheep, and goats, as well as other animals such as poultry and fish. This invention is of special importance to mammals. The usefulness of these complexes for vaccine production lies in the fact that the proteins are on the surface of the cell wall and are accessible to the medium surrounding the bacterial cells, so that the antigenic part of the chimeric protein is accessible to the antigen processing system. It is well known that presenting antigens in particulate form greatly enhances the immune response. In effect, bacteria containing antigenic peptides on the surfaces linked to the bacteria by these covalent interactions function as natural adjuvants. Here follows a representative list of typical microorganisms that express polypeptide antigens against which useful antibodies can be prepared by the methods of the present invention:

(1) Fungi: Candida albicans, Aspergillus fumigatus, Histoplasma capsulatum (all cause disseminating disease), Microsporum canis (animal ringworm).

(2) Parasitic protozoa: (1) Plasmodium falciparum (malaria), Trypanosoma cruzei (sleeping sickness).

(3) Spirochetes: (1) Borrelia bergdorferi (Lyme disease), Treponema pallidum (syphilis), Borrelia recurrentis (relapsing fever), Leptospira icterohaemorrhagiae (leptospirosis).

(4) Bacteria: Neisseria gonorrhoeae (gonorrhea), Staphylococcus aureus (endocarditis), Streptococcus pyogenes (rheumatic fever), Salmonella typhosa (salmonellosis), Hemophilus influenzae (influenza), Bordetella pertussis (whooping cough), Actinomyces israelii (actinomycosis), Streptococcus mutans (dental caries), Streptococcus egui (strangles in horses), Streptococcus agalactiae (bovine mastitis), Streplococcus anginosus (canine genital infections).

(5) Viruses: Human immunodeficiency virus (HIV), poliovirus, influenza virus, rabies virus, herpes virus, foot and mouth disease virus, psittacosis virus, paramyxovirus, myxovirus, coronavirus.

Additionally responses to nonmicrobial organisms may be evoked using polypeptide antigens. Tumor-associated antigens or other disease-associated antigens may be targeted. Typically, the resulting immunological response occurs by both humoral and cell-mediated pathways. One possible immunological response is the production of antibodies, thereby providing protection against infection by the pathogen.

This method is not limited to protein antigens. As discussed below, nonprotein antigens or haptens can be covalently linked to the C-terminal cell-wall targeting segment, which can be produced as an independently expressed polypeptide, either alone, or with a spacer at its amino-terminal end. If a spacer at the amino-terminal end is used, typically the spacer will have a conformation allowing the efficient interaction of the nonprotcin antigen or hapten with the immune system, most typically a random coil or a-helical form. The spacer can be of any suitable length; typically, it is in the range of about 5 to about 30 amino acids; most typically, about 10 to about 20 amino acids. In this version of the embodiment, the independently expressed polypeptide, once expressed, can then be covalently linked to the hapten or non-protein antigen. Typical non-protein antigens or haptens include drugs, including both drugs of abuse and therapeutic drugs, alkaloids, steroids, carbohydrates, aromatic compounds, including many pollutants, and other compounds that can be covalently linked to protein and against which an immune response can be raised.

Alternatively, a protein antigen can be covalently linked to the independently expressed cell-wall targeting segment or a cell-wall targeting segment including a spacer. Many methods for covalent linkage of both protein and non-protein compounds to proteins are well known in the art and are described, for example, in P. Tijssen, 1985, pp. 221-295, and in S. S. Wong, 1993.

Many reactive groups on both protein and non-protein compounds are available for conjugation. For example, organic moieties containing carboxyl groups or that can be carboxylated can be conjugated to proteins via the mixed anhydride method, the carbodiimide method, using dicyclohexylcarbodiimide, and the N-hydroxysuccinimide ester method. If the organic moiety contains amino groups or reducible nitro groups or can be substituted with such groups, conjugation can be achieved by one of several techniques. Aromatic amines can be converted to diazonium salts by the slow addition of nitrous acid and then reacted with proteins at a pH of about 9. If the organic moiety contains aliphatic amines, such groups can be conjugated to proteins by various methods, including carbodiimide, tolylene-2,4-diisocyanate, or malemide compounds, particularly the N-hydroxysuccinimide esters of malemide derivatives. An example of such a compound is 4-(N-maleimidomethyl)cyclohexane-1-carboxylic acid. Another example is m-maleimidobenzoyl-Nhydroxysuccinimide ester. Still another reagent that can be used is N-succinimidyl-3-(2pyridyldithio) propionate. Also, bifunctional esters, such as dimethylpimelimidate, dimethyladipimidate, or dimethylsuberimidate, can be used to couple amino-group containing moieties to proteins.

Additionally, aliphatic amines can also be converted to aromatic amines by reaction with e-nitrobenzoylchloride and subsequent reduction to a p-aminobenzoylamide, which can then be coupled to proteins after diazotization.

Organic moieties containing hydroxyl groups can be cross-linked by a number of indirect procedures. For example, the conversion of an alcohol moiety to the half ester of succinic acid (hemisuccinate) introduces a carboxyl group available for conjugation. The bifunctional reagent sebacoyldichloride converts alcohol to acid chloride which, at pH 9.5, reacts readily with proteins. Hydroxyl-containing organic moieties can also be conjugated through the highly reactive chlorocarbonates, prepared with an equal molar amount of phosgene.

For organic moieties containing ketones or aldehydes, such carbonyl containing groups can be derivatized into carboxyl groups through the formation of O(carboxymethyl) oximes. Ketone groups can also be derivatized with p-hydrazinobenzoic acid to produce carboxyl groups that can be conjugated to the specific binding partner as described above. Organic moieties containing aldehyde groups can be directly conjugated through the formation of Schiff bases which are then stabilized by a reduction with sodium borohydride.

One particularly useful cross-linking agent for hydroxyl-containing organic moieties is a photosensitive noncleavable heterobifunctional cross-linking reagent, sulfosuccinimidyl 6-[4′-azido-2′-nitrophenylamino] hexanoate. Other similar reagents are described in S. S. Wong, “Chemistry of Protein Conjugation and Cross-Linking,” supra.

Other cross-linking reagents can be used that introduce spacers between the organic moiety and the specific binding partner.

### VII. PRODUCTION OF SUBSTANTIALLY PURIFIED SORTASE-TRANSAMIDASE ENZYME

Another aspect of the present invention is methods for the production of substantially purified sortase-transamidase enzyme.

A. Methods Involving Expression of Cloned Gene

One method for the production of substantially purified sortase-transamidase enzyme involves the expression of the cloned gene, such as the srtA gene or the srtB gene. The isolation of the nucleic acid segment or segments encoding the sortase-transamidase enzyme is described above; these nucleic acid segment or segments are then incorporated into a vector and then use to transform a host in which the enzyme can be expressed. In one alternative, the host is a Gram-positive bacterium.

The next step in this alternative is expression in a Gram-positive bacterium to generate the cloned sortase-transamidase enzyme. Expression is typically under the control of various control elements associated with the vector incorporating the DNA encoding the sortase-transamidase gene. such as the coding region of the srtA gene; such elements can include promoters and operators, which can be regulated by proteins such as repressors. The conditions required for expression of cloned proteins in gram-positive bacteria, particularly S. aureus, are well known in the art and need not be further recited here. An example is the induction of expression of lysostaphin under control of the B1aZRI regulon induced by the addition of methicillin.

When expressed in Staphylococcus aureus, the chimeric protein is typically first exported with an amino-terminal leader peptide, such as the hydrophobic signal peptide at the amino-terminal region of the cloned lysostaphin of Recsei et al., 1987.

Alternatively, the cloned nucleic acid segment encoding the sortasetransamidase enzyme can be inserted in a vector that contains sequences allowing expression of the sort ase-transamidase in another organism, such as E. coli or S. lyphimurium. A suitable host organism can then be transformed or transfected with the vector containing the cloned nucleic acid segment. Expression is then performed in that host organism. The expressed enzyme is then purified using standard techniques. Techniques for the purification of cloned proteins are well known in the art and need not be detailed further here. One particularly suitable method of purification is affinity chromatography employing an immobilized antibody to sortase. Other protein purification methods include chromatography on ion-exchange resins, gel electrophoresis, isoelectric focusing, and gel filtration, among others.

One particularly useful form of affinity chromatography for purification of cloned proteins, such as sortase-transamidase, as well as other proteins, such as glutathione Stransferase and thioredoxin, that have been extended with carboxyl-terminal histidine residues, is chromatography on a nickel-sepharose column. This allows the purification of a sortase-transamidase enzyme extended at its carboxyl terminus with a sufficient number of bistidine residues to allow specific binding of the protein molecule to the nickel-sepharose column through the histidine residues. The bound protein is then eluted with imidazole. Typically, six or more histidine residues are added; preferably, six histidine residues are added. One way of adding the histidine residues to a cloned protein, such the sortasetransamidase, is through PCR with a primer that includes nucleotides encoding the histidine residues. The histidine codons are CAU and CAC expressed as RNA, which are CAT and CAC as DNA. Amplification of the cloned DNA with appropriate primers will add the histidine residues to yield a new nucleic acid segment, which can be recloned into an appropriate host for expression of the enzyme extended with the histidine residues.

A. Other Methods

Alternatively, the sortase-transamidase can be purified from Gram-positive bacteria by standard methods, including precipitation with reagents such as ammonium sulfate or protamine sulfate, ion-exchange chromatography, gel filtration chromatography, affinity chromatography, isoelectric focusing, and gel electrophoresis, as well as other methods known in the art.

Because the sortase-transamidase is a cysteine protease, one particularly useful method of purification involves covalent chromatography by thiol-disulfide interchange, using a two-protonic-state gel containing a 2-mcrcaptopyridine leaving group, such as Sepharose 2B-glutathione 2-pyridyl disulfide or Sepharose 6B-hydroxypropyl 2-pyridyl disulfide. Such covalent chromatographic techniques are described in Brocklehurst et al., 1987, ch. 2.

### VIII. FURTHER APPLICATIONS OF SORTASE-TRANSAMIDASE

A. Production of Antibodies

Antibodies can be prepared to the substantially purified sortase-transamidase of the present invention, whether the sortase-transamidase is purified from bacteria or produced from recombinant bacteria as a result of gene cloning procedures. Because the substantially purified enzyme according to the present invention is a protein, it is an effective antigen, and antibodies can be made by well-understood methods such as those disclosed in E. Harlow & D. Lane, 1988. In general, antibody preparation involves immunizing an antibody-producing animal with the protein, with or without an adjuvant such as Freund's complete or incomplete adjuvant, and purification of the antibody produced. The resulting polyclonal antibody can be purified by techniques such as affinity chromatography.

Once the polyclonal antibodies are prepared, monoclonal antibodies can be prepared by standard procedures, such as those described in Chapter 6 of Harlow & Lane, supra.

B. Derivatives for Affinity Chromatography

Another aspect of the present invention is derivatives of the cloned, substantially purified sortase-transamidase of the present invention extended at its carboxyl terminus with a sufficient number of histidinc residues to allow specific binding of the protein molecule to a nickel-sepharose column through the histidine residues. Typically, six or more histidine residues are added; preferably, six histidine residues are added. The histidine residues can be added to the carboxyl terminus through PCR cloning as described above. Additional detail for compositions and methods that may be employed with methods described above are provided.

### IX. PROTEINACEOUS COMPOSITIONS

In certain embodiments, the present invention concerns novel compositions comprising at least one proteinaceous molecule, and it concerns methods involving proteinaceous molecules. Proteinaceous molecules of the invention include, but are not limited to, a sortase-transamidase, a peptide or polypeptide with a sorting signal, an antibody used to detect a cleaved peptide or polypeptide that contained a sorting signal prior to being cleaved, a label or detection reagent, a candidate compound that may modulate the activity or expression of a sortase-transamidase, an antigen, a vaccine, or a conjugate. Such proteinaceous molecules may be used in treatment, prevention, screening, production, and expression methods of the invention.

In many embodiments of the invention, a polypeptide having sortase-transamidase activity is employed, such as Srt A and/or Srt B; other sortases known to those of skill in the art may also be employed. A polypeptide with a sorting signal is also employed in several aspects of the invention. The sorting signal may include all or part of the sequences NPQ/KTN/G or LPX₃X₄G, where X3 is any of the 20 naturally occurring amino acid and X₄ is an alanine, serine, or threonine. The sorting signal may also include all or part of SEQ ID NO:2, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, or SEQ ID NO:33.

As used herein, a “proteinaceous molecule,” “proteinaceous composition,” “proteinaceous compound,” “proteinaceous chain” or “proteinaceous material” generally refers, but is not limited to, a protein of greater than about 200 amino acids or the full length endogenous sequence translated from a gene; a polypeptide of greater than about 100 amino acids; and/or a peptide of from about 3 to about 100 amino acids. All the “proteinaceous” terms described above may be used interchangeably herein.

In certain embodiments the size of the at least one proteinaceous molecule may comprise, but is not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 or greater amino molecule residues, and any range derivable therein. Such a molecule may comprise, be at least, or be at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300 or more contiguous amino acids of SEQ ID NO:2, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, or SEQ ID NO:33. Further, it is contemplated that proteinaceous compounds may include, for example, five contiguous amino acids from one part of a SEQ ID NO and additional contiguous amino acids from another part of the same or different SEQ ID NO. Spacing between sets of contiguous amino acids may or may not be maintained.

As used herein, an “amino molecule” refers to any amino acid, amino acid derivative or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

Accordingly, the term “proteinaceous composition” encompasses amino molecule sequences comprising at least one of the 20 common amino acids in naturally synthesized proteins, or at least one modified or unusual amino acid, including but not limited to those shown on Table 2 below.

TABLE 2
Modified and Unusual Amino Acids

| Abbr. | Amino Acid |
|---|---|
| Aad | 2-Aminoadipic acid |
| Baad | 3-Aminoadipic acid |
| Bala | β-alanine, β-Amino-propionic acid |
| Abu | 2-Aminobutyric acid |
| 4Abu | 4-Aminobutyric acid, piperidinic acid |
| Acp | 6-Aminocaproic acid |
| Ahe | 2-Aminoheptanoic acid |
| Aib | 2-Aminoisobutyric acid |
| Baib | 3-Aminoisobutyric acid |
| Apm | 2-Aminopimelic acid |
| Dbu | 2,4-Diaminobutyric acid |
| Des | Desmosine |
| Dpm | 2,2′-Diaminopimelic acid |
| Dpr | 2,3-Diaminopropionic acid |
| EtGly | N-Ethylglycine |
| EtAsn | N-Ethylasparagine |
| Hyl | Hydroxylysine |
| AHyl | allo-Hydroxylysine |
| 3Hyp | 3-Hydroxyproline |
| 4Hyp | 4-Hydroxyproline |
| Ide | Isodesmosine |
| AIle | allo-Isoleucine |
| MeGly | N-Methylglycine, sarcosine |
| MeIle | N-Methylisoleucine |
| MeLys | 6-N-Methyllysine |
| MeVal | N-Methylvaline |
| Nva | Norvaline |
| Nle | Norleucine |
| Orn | Ornithine |

In certain embodiments the proteinaceous composition comprises at least one protein, polypeptide or peptide. In further embodiments the proteinaceous composition comprises a biocompatible protein, polypeptide or peptide. As used herein, the term “biocompatible” refers to a substance which produces no significant untoward effects when applied to, or administered to, a given organism according to the methods and amounts described herein. Such untoward or undesirable effects are those such as significant toxicity or adverse immunological reactions. In preferred embodiments, biocompatible protein, polypeptide or peptide containing compositions will generally be mammalian proteins or peptides or synthetic proteins or peptides each essentially free from toxins, pathogens and harmful immunogens.

Also within the scope of the present invention are substantially purified protein molecules that are mutants of the sequence of SEQ ID NO:2 or SEQ ID NO:4 that preserve the sortase-transamidase activity. In particular, the conservative amino acid substitutions can be any of the following: (1) any of isoleucine for leucine or valine, leucine for isoleucine, and valine for leucine or isoleucine; (2) aspartic acid for glutamic acid and glutamic acid for aspartic acid; (3) glutamine for asparagine and asparagine for glutamine; and (4) serine for threonine and threonine for serine.

Other substitutions can also be considered conservative, depending upon the environment of the particular amino acid. For example, glycine (G) and alanine (A) can frequently be interchangeable, as can be alanine and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the different pK's of these two amino acid residues or their different sizes are not significant. Still other changes can be considered “conservative” in particular environments. For example, if an amino acid on the surface of a protein is not involved in a hydrogen bond or salt bridge interaction with another molecule, such as another protein subunit or a ligand bound by the protein, negatively charged amino acids such as glutamic acid and aspartic acid can be substituted for by positively charged amino acids such as lysine or arginine and vice versa. Histidine (H), which is more weakly basic than arginine or lysine, and is partially charged at neutral pH, can sometimes be substituted for these more basic amino acids. Additionally, the amides glutamine (Q) and asparagine (N) can sometimes be substituted for their carboxylic acid homologues, glutamic acid and aspartic acid.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. The nucleotide and protein, polypeptide and peptide sequences for various genes have been previously disclosed, and may be found at computerized databases known to those of ordinary skill in the art. One such database is the National Center for Biotechnology Information's Genbank and GenPept databases (http://www.ncbi.nlm.nih.gov/). The coding regions for these known genes may be amplified and/or expressed using the techniques disclosed herein or as would be know to those of ordinary skill in the art. Alternatively, various commercial preparations of proteins, polypeptides and peptides are known to those of skill in the art.

In certain embodiments a proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

In certain embodiments, the proteinaceous composition may comprise at least one antibody. It is contemplated that antibodies can be used to detect polypeptides, such as polypeptides cleaved by a sortase-transamidase and displayed on the surface of a bacterium. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting. An antibody may be polyclonal or monoclonal; it may be humanized or be a single-chain antibody.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

It is contemplated that virtually any protein, polypeptide or peptide containing component may be used in the compositions and methods disclosed herein. However, it is preferred that the proteinaceous material is biocompatible. In certain embodiments, it is envisioned that the formation of a more viscous composition will be advantageous in that will allow the composition to be more precisely or easily applied to the tissue and to be maintained in contact with the tissue throughout the procedure. In such cases, the use of a peptide composition, or more preferably, a polypeptide or protein composition, is contemplated. Ranges of viscosity include, but are not limited to, about 40 to about 100 poise. In certain aspects, a viscosity of about 80 to about 100 poise is preferred.

Proteins and peptides suitable for use in this invention may be autologous proteins or peptides, although the invention is clearly not limited to the use of such autologous proteins. As used herein, the term “autologous protein, polypeptide or peptide” refers to a protein, polypeptide or peptide which is derived or obtained from an organism. Organisms that may be used include, but are not limited to, a bovine, a reptilian, an amphibian, a piscine, a rodent, an avian, a canine, a feline, a fungal, a plant, or a prokaryotic organism, with a selected animal or human subject being preferred. The “autologous protein, polypeptide or peptide” may then be used as a component of a composition intended for application to the selected animal or human subject. In certain aspects, the autologous proteins or peptides are prepared, for example from whole plasma of the selected donor. The plasma is placed in tubes and placed in a freezer at about −80° C. for at least about 12 hours and then centrifuged at about 12,000 times g for about 15 minutes to obtain the precipitate. The precipitate, such as fibrinogen may be stored for up to about one year (Oz, 1990).

In certain embodiments, it will be advantageous to employ proteinaceous compositions in combination with an appropriate means, such as a label, for determining the presence of a particular compound, such as a polypeptide. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, calorimetric indicator substrates are known that can be employed to provide a detection means that is visibly or spectrophotometrically detectable, to identify specific binding to a targeted compound whose presence or activity is being assayed.

A. Immunological Reagents

In certain aspects of the invention, one or more antibodies against a proteinaceous compound of the invention may be desirable. These antibodies may be used in various diagnostic, therapeutic, preventative, or screening applications, described herein below. An antibody can be used to detect the presence of a particular antigen or polypeptide. An antibody to a sortase-transamidase may be used to identify, characterize, or purify the sortase-transamidase. Alternatively, in other aspects of the invention, a compound that elicits or induces an immune response is desirable for administration to an organism to elicit an immune response against the compound in the organism.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting. As used herein, the term “antigen” refers to substance that elicits an immune response in an organism.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

Monoclonal antibodies (MAbs) are recognized to have certain advantages, e.g., reproducibility and large-scale production, and their use is generally preferred. The invention thus provides monoclonal antibodies of the human, murine, monkey, rat, hamster, rabbit and even chicken origin. Due to the ease of preparation and ready availability of reagents, murine monoclonal antibodies will often be preferred.

However, “humanized” antibodies are also contemplated, as are chimeric antibodies from mouse, rat, or other species, bearing human constant and/or variable region domains, bispecific antibodies, recombinant and engineered antibodies and fragments thereof.

A wide range of animal species can be used for the production of antisera. Typically the animal used for production of antisera is a rabbit, a mouse, a rat, a hamster, a guinea pig or a goat. The choice of animal may be decided upon the ease of manipulation, costs or the desired amount of sera, as would be known to one of skill in the art.

As is also well known in the art, the immunogenicity of a particular immunogen composition can be enhanced by the use of non-specific stimulators of the immune response, known as adjuvants. Suitable adjuvants include all acceptable immunostimulatory compounds, such as cytokines, chemokines, cofactors, toxins, plasmodia op synthetic compositions.

Adjuvants that may be used include IL-1, IL-2, IL-4, IL-7, IL-12, γ-interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion is also contemplated. MHC antigens may even be used. Exemplary, often preferred adjuvants include complete Freund's adjuvant (a non-specific stimulator of the immune response containing killed Mycobacterium tuberculosis), incomplete Freund's adjuvants and aluminum hydroxide adjuvant.

In addition to adjuvants, it may be desirable to coadminister biologic response modifiers (BRM), which have been shown to upregulate T cell immunity or downregulate suppressor cell activity. Such BRMs include, but are not limited to, Cimetidine (CIM; 1200 mg/d) (Smith/Kline, PA); low-dose Cyclophosphamide (CYP; 300 mg/m²) (Johnson/Mead, NJ), cytokines such as γ-interferon, IL-2, or IL-12 or genes encoding proteins involved in immune helper functions, such as B-7.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified protein, polypeptide, peptide or domain, be it a wild-type or mutant composition. The immunizing composition is administered in a manner effective to stimulate antibody producing cells.

It is also contemplated that a molecular cloning approach may be used to generate monoclonals. In one embodiment, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 10⁴ times as many antibodies can be produced and screened in a single round, and that new specificities are generated by H and L chain combination which further increases the chance of finding appropriate antibodies. In another example, LEEs or CEEs can be used to produce antigens in vitro with a cell free system. These can be used as targets for scanning single chain antibody libraries. This would enable many different antibodies to be identified very quickly without the use of animals.

Alternatively, monoclonal antibody and antigenic fragments encompassed by the present invention can be synthesized using an automated peptide synthesizer, or by expression of full-length gene or of gene fragments in E. coli.

In other embodiments, derivatization of immunoglobulins by selectively introducing sulfhydryl groups in the Fe region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fe region have also been disclosed in the literature (O'Shannessy et al., 1987). This approach has been reported to produce diagnostically and therapeutically promising antibodies which are currently in clinical evaluation.

B. Conjugates, Including Antibody Conjugates

The present invention further provides polypeptides, including antigens and antibodies against translated proteins, polypeptides and peptides, generally of the monoclonal type, that may be linked to at least one agent to form a conjugate. In order to increase the efficacy of proteinaceous molecules as screening or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules, which have been attached to antibodies, include toxins, anti-tumor agents, antiobiotics, therapeutic enzymes, radio-labeled nucleotides, antiviral agents, chelating agents, cytokines, growth factors, and oligo- or poly-nucleotides. By contrast, a label or a detection agent is defined as any moiety that may be detected using an assay. Non-limiting examples of labels or detection reagents that have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin. The examples that involve detection by color are generally understood to be colorimetric labels or detection reagents. Herein, “label” and “detection reagent” are used interchangeably.

Antibodies have been the main focus of protein conjugates and are discussed below. However, the examples of antibody conjugates may be applied more generally to any proteinaceous composition described herein. Thus, the teachings with respect to antibody conjugates may be applied to a polypeptide that comprises a sorting signal.

Any antibody of sufficient selectivity, specificity or affinity may be employed as the basis for an antibody conjugate. Such properties may be evaluated using conventional immunological screening methodology known to those of skill in the art. Sites for binding to biological active molecules in the antibody molecule, in addition to the canonical antigen binding sites, include sites that reside in the variable domain that can bind pathogens, B-cell superantigens, the T cell co-receptor CD4 and the HIV-1 envelope (Sasso et al., 1989; Shorki et al., 1991; Silvermann et al., 1995; Cleary et al., 1994; Lenert et al., 1990; Berberian et al., 1993; Kreier et al., 1991). In addition, the variable domain is involved in antibody self-binding (Kang et al., 1988), and contains epitopes (idiotopes) recognized by anti-antibodies (Kohler et al., 1989).

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to which they are attached to be detected, and/or further quantified if desired. Another such example is the formation of a conjugate comprising an antibody linked to a cytotoxic or anti-cellular agent, and may be termed “immunotoxins”.

Antibody conjugates are generally preferred for use as diagnostic agents. Antibody diagnostics generally fall within two classes, those for use in in vitro diagnostics, such as in a variety of immunoassays, and/or those for use in vivo diagnostic protocols, generally known as “antibody-directed imaging”.

Many appropriate imaging agents are known in the art, as are methods for their attachment to antibodies (see, for e.g., U.S. Pat. Nos. 5,021,236; 4,938,948; and 4,472,509, each incorporated herein by reference). The imaging moieties used can be paramagnetic ions; radioactive isotopes; fluorochromes; NMR-detectable substances; X-ray imaging.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred for use in certain embodiments, and technicium^{99m} and/or indium¹¹¹ are also often preferred due to their low energy and suitability for long range detection. Radioactively labeled monoclonal antibodies of the present invention may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the invention may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Another type of antibody conjugates contemplated in the present invention are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter & Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; and Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

1. Linkers/Coupling Agents

If desired, compounds may be joined with other compounds of the inention. A therapeutic, preventative, or screening compound may be joined via a biologically-releasable bond, such as a selectively-cleavable linker or amino acid sequence. For example, peptide linkers that include a cleavage site for an enzyme preferentially located or active within a particular environment are contemplated. Exemplary forms of such peptide linkers are those that are cleaved by urokinase, plasmin, thrombin, Factor IXa, Factor Xa, or a metallaproteinase, such as collagenase, gelatinase, or stromelysin.

Amino acids such as selectively-cleavable linkers, synthetic linkers, or other amino acid sequences may be used to separate a compounds from one another.

Additionally, while numerous types of disulfide-bond containing linkers are known that can successfully be employed to conjugate compounds, such as an antiobiotic to a polypeptide or a label to a polypeptide, certain linkers will generally be preferred over other linkers, based on differing pharmacologic characteristics and capabilities. For example, linkers that contain a disulfide bond that is sterically “hindered” are to be preferred, due to their greater stability in vivo, thus preventing release of the toxin moiety prior to binding at the site of action. Furthermore, while certain advantages in accordance with the invention will be realized through the use of any of a number of toxin moieties, the inventors have found that the use of ricin A chain, and even more preferably deglycosylated A chain, will provide particular benefits.

a. Biochemical Cross-Linkers

The joining of any of the above components, to targeting peptide will generally employ the same technology as developed for the preparation of immunotoxins. It can be considered as a general guideline that any biochemical cross-linker that is appropriate for use in an immunotoxin will also be of use in the present context, and additional linkers may also be considered.

Cross-linking reagents are used to form molecular bridges that tie together functional groups of two different molecules, e.g., a stablizing and coagulating agent. To link two different proteins in a step-wise manner, hetero-bifunctional cross-linkers can be used that eliminate unwanted homopolymer formation.

TABLE 3
HETERO-BIFUNCTIONAL CROSS-LINKERS

| | | | Spacer Arm | |
|---|---|---|---|---|
| | | | Length\after cross- | |
| linker | Reactive Toward | Advantages and Applications | linking | |
| SMPT | Primary amines | Greater stability | 11.2 | A |
| | Sulfhydryls | | | |
| SPDP | Primary amines | Thiolation | 6.8 | A |
| | Sulfhydryls | Cleavable cross-linking | | |
| LC-SPDP | Primary amines | Extended spacer arm | 15.6 | A |
| | Sulfhydryls | | | |
| Sulfo-LC-SPDP | Primary amines | Extended spacer arm | 15.6 | A |
| | Sulfhydryls | Water-soluble | | |
| SMCC | Primary amines | Stable maleimide reactive group | 11.6 | A |
| | Sulfhydryls | Enzyme-antibody conjugation | | |
| | | Hapten-carrier protein conjugation | | |
| Sulfo-SMCC | Primary amines | Stable maleimide reactive group | 11.6 | A |
| | Sulfhydryls | Water-soluble | | |
| | | Enzyme-antibody conjugation | | |
| MBS | Primary amines | Enzyme-antibody conjugation | 9.9 | A |
| | Sulfhydryls | Hapten-carrier protein conjugation | | |
| Sulfo-MBS | Primary amines | Water-soluble | 9.9 | A |
| | Sulfhydryls | | | |
| SIAB | Primary amines | Enzyme-antibody conjugation | 10.6 | A |
| | Sulfhydryls | | | |
| Sulfo-SIAB | Primary amines | Water-soluble | 10.6 | A |
| | Sulfhydryls | | | |
| SMPB | Primary amines | Extended spacer arm | 14.5 | A |
| | Sulfhydryls | Enzyme-antibody conjugation | | |
| Sulfo-SMPB | Primary amines | Extended spacer arm | 14.5 | A |
| | Sulfhydryls | Water-soluble | | |
| EDC/Sulfo-NHS | Primary amines | Hapten-Carrier conjugation | 0 | |
| | Carboxyl groups | | | |
| ABH | Carbohydrates | Reacts with sugar groups | 11.9 | A |
| | Nonselective | | | |

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It can therefore be seen that a targeted peptide composition will generally have, or be derivatized to have, a functional group available for cross-linking purposes. This requirement is not considered to be limiting in that a wide variety of groups can be used in this manner. For example, primary or secondary amine groups, hydrazide or hydrazine groups, carboxyl alcohol, phosphate, or alkylating groups may be used for binding or cross-linking. For a general overview of linking technology, one may wish to refer to Ghose & Blair (1987).

The spacer arm between the two reactive groups of a cross-linkers may have various length and chemical compositions. A longer spacer arm allows a better flexibility of the conjugate components while some particular components in the bridge (e.g., benzene group) may lend extra stability to the reactive group or an increased resistance of the chemical link to the action of various aspects (e.g., disulfide bond resistant to reducing agents). The use of peptide spacers, such as L-Leu-L-Ala-L-Leu-L-Ala, is also contemplated.

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagents for use in immunotoxins is SMPT, which is a bifunctional cross-linker containing a disulfide bond that is “sterically hindered” by an adjacent benzene ring and methyl groups. It is believed that stearic hindrance of the disulfide bond serves a function of protecting the bond from attack by thiolate anions such as glutathione which can be present in tissues and blood, and thereby help in preventing decoupling of the conjugate prior to the delivery of the attached agent to the tumor site. It is contemplated that the SMPT agent may also be used in connection with the bispecific coagulating ligands of this invention.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art.

Once conjugated, the polypeptide generally will be purified to separate the conjugate from unconjugated compounds and from other contaminants. A large a number of purification techniques are available for use in providing conjugates of a sufficient degree of purity to render them clinically useful. Purification methods based upon size separation, such as gel filtration, gel permeation or high performance liquid chromatography, will generally be of most use. Other chromatographic techniques, such as Blue-Sepharose separation, may also be used.

In addition to chemical conjugation, a purified proteinaceous compound may be modified at the protein level. Included within the scope of the invention are protein fragments or other derivatives or analogs that are differentially modified during or after translation, for example by glycosylation, acetylation, phosphorylation, amidation, derivatization by known protecting/blocking groups, and proteolytic cleavage. Any number of chemical modifications may be carried out by known techniques, including but not limited to specific chemical cleavage by cyanogen bromide, trypsin, chymotrypsin, papain, V8 protease, NaBH₄; acetylation, formylation, farnesylation, oxidation, reduction; metabolic synthesis in the presence of tunicamycin.

C. Chimeric Polypeptides and Proteins

In accordance with the objects of the present invention, a polynucleotide that encodes a chimeric protein, mutant polypeptide, biologically active fragment of chimeric protein, or functional equivalent thereof, may be used to generate recombinant DNA molecules that direct the expression of the chimeric protein, chimeric peptide fragments, or a functional equivalent thereof, in appropriate host cells. A chimeric protein or polypeptide is characterized by an amino acid sequence not normally found in nature. Such a protein or polypeptide generally has an amino acid sequence from more than one protein or polypeptide or from the same protein or polypeptide but from a different species of organism. A chimeric protein may have sequences from one polypeptide inserted into a second polypeptide recombinantly.

D. Fusion Proteins

A specialized kind of insertional variant of a chimeric protein is the fusion protein. Fusion proteins are contemplated as part of the invention. In some embodiments, a sorting signal from a polypeptide of a Gram-positive bacterium may be combined with an amino acid sequence that is lacking such a sorting signal. This molecule may have all or part of one proteinaceous molecule, linked at the N- or C-terminus, to all or a portion of a second polypeptide. Other general examples include fusions that typically employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes such as a hydrolase, glycosylation domains, cellular targeting signals or transmembrane regions. Additionally, a proteinaceous label may be placed onto the end of a polypeptide. Fusions may be generated recombinantly, as distinguished from protein conjugates, which are chemically generated. The use of recombinant DNA techniques to achieve such ends is now standard practice to those of skill in the art. These methods include, for example, in vitro recombinant DNA techniques, synthetic techniques and in vivo recombination/genetic recombination. DNA and RNA synthesis may, additionally, be performed using an automated synthesizers (see, for example, the techniques described in Sambrook et al., 1989; and Ausubel et al., 1989).

The preparation of such a fusion protein generally entails the preparation of a first and second DNA coding region and the functional ligation or joining of said regions, in frame, to prepare a single coding region that encodes the desired fusion protein. In the present context, a sorting signal DNA sequence will generally be joined in frame with a DNA sequence encoding a polypeptide desired to be cleaved by a sortase-transamidase and/or to be expressed on the surface of a Gram-positive bacterium.

Once the coding region desired has been produced, an expression vector is created. Expression vectors contain one or more promoters upstream of the inserted DNA regions that act to promote transcription of the DNA and to thus promote expression of the encoded recombinant protein. This is the meaning of “recombinant expression” and has been discussed elsewhere in the specification.

### X. PROTEIN PURIFICATION

To prepare a composition comprising a sortase-transamidase or a polypeptide with a sorting signal, it may be desirable to purify the components or variants thereof. Other proteinaceous compounds described herein may also be purified. According to one embodiment of the present invention, purification of sortase-transamidase can be utilized in a variety of methods of the invention. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide, such as a sortase-transamidase. The term “purified protein or peptide” as used herein is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition, such as the dehydrogenase, that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (e.g., alter pH, ionic strength, and temperature.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand also should provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

A. Synthetic Peptides

The present invention also describes a sorting signal for use in various embodiments of the present invention. In some aspects of the invention, peptides with a sorting signal motif are employed to compete effectively as substrates for sortase-transamidases as a therapeutic or diagnostic agent. The peptides of the invention can be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Peptides with at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 50, 55, 60, 65, 70, 75, 80, 85, 90, 95 or up to about 100 amino acid residues are contemplated by the present invention.

The compositions of the invention may include a peptide comprising a sorting sequence that has been modified to enhance its activity or to render it biologically protected. Biologically protected peptides have certain advantages over unprotected peptides when administered to human subjects and, as disclosed in U.S. Pat. No. 5,028,592, incorporated herein by reference, protected peptides often exhibit increased pharmacological activity.

Compositions for use in the present invention may also comprise peptides that include all L-amino acids, all D-amino acids, or a mixture thereof. The use of D-amino acids may confer additional resistance to proteases naturally found within the human body and are less immunogenic and can therefore be expected to have longer biological half lives.

### XI. PROTEIN ASSAYS

Assays to detect or characterize a a proteinaceous compound are well known to those of skill in the art. In some cases the proteinaceous compound to be detected is labeled or attached to a detection reagent. Other immunodetection methods are disclosed below.

In still further embodiments, the present invention concerns immunodetection methods for binding, purifying, removing, quantifying and/or otherwise generally detecting biological components such as dehydrogenase-expressed message(s), protein(s), polypeptide(s) or peptide(s). Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle M H and Ben-Zeev O, 1999; Gulbis B and Galand P, 1993; De Jager R et al., 1993; and Nakamura et al., 1987, each incorporated herein by reference.

In general, the immunobinding methods include obtaining a sample suspected of containing dehydrogenase expressed message and/or protein, polypeptide and/or peptide, and contacting the sample with an antibody in accordance with the present invention, as the case may be, under conditions effective to allow the formation of immunocomplexes.

These methods include methods for purifying a sortase-transamidase or a polypeptide containing a sorting signal message, protein, polypeptide and/or peptide from organelle, cell, tissue or organism's samples. In these instances, the antibody removes the antigenic message, protein, polypeptide and/or peptide component from a sample. The antibody will preferably be linked to a solid support, such as in the form of a column matrix, and the sample suspected of containing the message, protein, polypeptide and/or peptide antigenic component will be applied to the immobilized antibody. The unwanted components will be washed from the column, leaving the antigen immunocomplexed to the immobilized antibody to be eluted.

The immunobinding methods also include methods for detecting and quantifying the amount of an antigen component in a sample and the detection and quantification of any immune complexes formed during the binding process. Here, one would obtain a sample suspected of containing an antigen, and contact the sample with an antibody against the dehydrogenase produced antigen, and then detect and quantify the amount of immune complexes formed under the specific conditions.

In terms of antigen detection, the biological sample analyzed may be any sample that is suspected of containing an antigen, such as, for example, a tissue section or specimen, a homogenized tissue extract, a cell, an organelle, separated and/or purified forms of any of the above antigen-containing compositions, or even any biological fluid that comes into contact with the cell or tissue, including blood and/or serum, although tissue samples or extracts are preferred.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any dehydrogenase antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The antigen antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection designed by Charles Cantor uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

The immunodetection methods of the present invention have evident utility in the diagnosis and prognosis of conditions such as various diseases wherein a specific dehydrogenase is expressed, such as an viral dehydrogenase of a viral infected cell, tissue or organism; a cancer specific gene product, etc. Here, a biological and/or clinical sample suspected of containing a specific disease associated dehydrogenase expression product is used. However, these embodiments also have applications to non-clinical samples, such as in the titering of antigen or antibody samples, for example in the selection of hybridomas.

In the clinical diagnosis and/or monitoring of patients with various forms a disease, such as, for example, macular or retinal degeneration, the detection of a macular specific gene product, and/or an alteration in the levels of a macular or retinal degeneration specific gene product, in comparison to the levels in a corresponding biological sample from a normal subject is indicative of a patient with macular or retinal degeneration. However, as is known to those of skill in the art, such a clinical diagnosis would not necessarily be made on the basis of this method in isolation. Those of skill in the art are very familiar with differentiating between significant differences in types and/or amounts of biomarkers, which represent a positive identification, and/or low level and/or background changes of biomarkers. Indeed, background expression levels are often used to form a “cut-off” above which increased detection will be scored as significant and/or positive. Of course, the antibodies of the present invention in any immunodetection or therapy known to one of ordinary skill in the art.

### XII. NUCLEIC ACID COMPOSITIONS

Certain embodiments of the present invention involve the synthesis, creation, and/or mutation of a nucleic acid molecule and recombinant vectors encoding one or more sortase-transamidases of SEQ ID NO: 2 or 4, or any other known sortase-transamidase, or one or more polypeptides comprising a sorting signal. For example, any polypeptide can be engineered for use with the present invention by incorporating a sorting signal that is recognized by a sortase-transamidase. Other embodiments may involve generating a bacterium that has been mutated such that the progeny bacterium expresses one fewer type of sortase-transamidase than their parents that were not mutated. Thus, a mutation may be introduced in the sortase-transamidase gene of a bacterium, such that the bacterium no longer expresses a function version of that sortase-transamidase. Embodiments of the invention also involve the creation and use of recombinant host cells through the application of DNA technology, that express one or more of the proteinaceous compounds described herein. In certain aspects, a nucleic acid encoding a sortase-transamidase or polypeptide having a sorting signal comprises a wild-type or a mutant nucleic acid. The nucleic acid compositions can, for example, be used to screen for inhibitors of sortase-transamidases or to express sortase-transamidases, or for generating a polypeptide with a sorting signal.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length.

These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule. As used herein, a single stranded nucleic acid may be denoted by the prefix “ss,” a double stranded nucleic acid by the prefix “ds,” and a triple stranded nucleic acid by the prefix “ts.”

A. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 1989, incorporated herein by reference).

B Purification of Nucleic Acids

A nucleic acid may be purified on polyacrylamide gels, cesium chloride centrifugation gradients, or by any other means known to one of ordinary skill in the art (see for example, Sambrook et al., 1989, incorporated herein by reference).

In certain aspect, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

C. Nucleic Acid Segments

In certain embodiments, the nucleic acid is a nucleic acid segment, such as one encoding a proteinaceous composition described herein. As used herein, the term “nucleic acid segment,” are smaller fragments of a nucleic acid, such as for non-limiting example, those that encode only part of the dehydrogenase peptide or polypeptide sequence. Thus, a “nucleic acid segment” may comprise any part of a gene sequence, of from about 2 nucleotides to the full length of the dehydrogenase peptide or polypeptide encoding region.

In a non-limiting example, nucleic acid segments may comprise or be limited to 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 4000, or 5000 nucleotides. Contiguous nucleic acids segments of SEQ ID NO: 1 and 3 may be used in the present invention, as well as any nucleic acid segment encoding any proteinaceous composition described herein such as a sortase-transamidase or any polypeptide that can have a sorting signal inserted into it. Nucleic acid segments may also contain up to 10,000, 20,000, 30,000, 50,000, 100,000, 250,000, 500,000, 750,000, to 1,000,000 nucleotides in length, as well as constructs of greater size, up to and including chromosomal sizes are contemplated for use in the present invention.

As used herein “wild-type” refers to the naturally occurring sequence of a nucleic acid at a genetic locus in the genome of an organism, or a sequence transcribed or translated from such a nucleic acid. Thus, the term “wild-type” also may refer to an amino acid sequence encoded by a nucleic acid. As a genetic locus may have more than one sequence or alleles in a population of individuals, the term “wild-type” encompasses all such naturally occurring allele(s). As used herein the term “polymorphic” means that variation exists (i.e., two or more alleles exist) at a genetic locus in the individuals of a population. As used herein “mutant” refers to a change in the sequence of a nucleic acid or its encoded protein, polypeptide or peptide that is the result of the hand of man.

The present invention also concerns the isolation or creation of a recombinant construct or a recombinant host cell through the application of recombinant nucleic acid technology known to those of skill in the art or as described herein. A recombinant construct or host cell may express a dehydrogenase protein, peptide or peptide, or at least one biologically functional equivalent thereof. The recombinant host cell may be a prokaryotic cell. In a more preferred embodiment, the recombinant host cell is a eukaryotic cell. As used herein, the term “engineered” or “recombinant” cell is intended to refer to a cell into which a recombinant gene, such as a gene encoding a dehydrogenase, has been introduced. Therefore, engineered cells are distinguishable from naturally occurring cells which do not contain a recombinantly introduced gene. Engineered cells are thus cells having a gene or genes introduced through the hand of man. Recombinantly introduced genes will either be in the form of a cDNA gene (i.e., they will not contain introns), a copy of a genomic gene, or will include genes positioned adjacent to a promoter not naturally associated with the particular introduced gene.

Herein certain embodiments, a “gene” refers to a nucleic acid that is transcribed. In certain aspects, the gene includes regulatory sequences involved in transcription, or message production or composition. In particular embodiments, the gene comprises transcribed sequences that encode for a protein, polypeptide or peptide, termed “coding sequence.” As will be understood by those in the art, this function term “gene” includes both genomic sequences, RNA or cDNA sequences or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express using nucleic acid manipulation technology, proteins, polypeptides, domains, peptides, fusion proteins, mutants and/or such like.

The nucleic acid(s) of the present invention, regardless of the length of the sequence itself, may be combined with other nucleic acid sequences, including but not limited to, promoters, enhancers, polyadenylation signals, restriction enzyme sites, multiple cloning sites, coding segments, and the like, to create one or more nucleic acid construct(s). As used herein, a “nucleic acid construct” is a nucleic acid engineered or altered by the hand of man, and generally comprises one or more nucleic acid sequences organized by the hand of man.

In a non-limiting example, one or more nucleic acid constructs may be prepared containing about 3, about 5, about 8, about 10 to about 14, or about 15, about 20, about 30, about 40, about 50, about 100, about 200, about 500, about 1,000, about 2,000, about 3,000, about 5,000, about 10,000, about 15,000, about 20,000, about 30,000, about 50,000, about 100,000, about 250,000, about 500,000, about 750,000, to about 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges”, as used herein, means any length or range including or between the quoted values (i.e., all integers including and between such values). Non-limiting examples of intermediate lengths include about 11, about 12, about 13, about 16, about 17, about 18, about 19, etc.; about 21, about 22, about 23, etc.; about 31, about 32, etc.; about 51, about 52, about 53, etc.; about 101, about 102, about 103, etc.; about 151, about 152, about 153, etc.; about 1,001, about 1002, etc; about 50,001, about 50,002, etc; about 750,001, about 750,002, etc.; about 1,000,001, about 1,000,002, etc. Non-limiting examples of intermediate ranges include about 3 to about 32, about 150 to about 500,001, about 3,032 to about 7,145, about 5,000 to about 15,000, about 20,007 to about 1,000,003, etc.

The term “functionally equivalent codon” is used herein to refer to codons that encode the same amino acid, such as the six codons for arginine and serine, and also refers to codons that encode biologically equivalent amino acids. Codon usage for various organisms and organelles can be found at the website http://www.kazusa.or.jp/codon/, incorporated herein by reference, allowing one of skill in the art to optimize codon usage for expression in various organisms using the disclosures herein. Thus, it is contemplated that codon usage may be optimized for other animals, as well as other organisms such as a prokaryote (e.g., an cubacteria, an archaea), an eukaryote (e.g., a protist, a plant, a fungi, an animal), a virus and the like, as well as organelles that contain nucleic acids, such as mitochondria, chloroplasts and the like, based on the preferred codon usage as would be known to those of ordinary skill in the art.

It will also be understood that amino acid sequences or nucleic acid sequences may include additional residues, such as additional N- or C-terminal amino acids or 5′ or 3′ sequences, or various combinations thereof, and yet still be essentially as set forth in one of the sequences disclosed herein, so long as the sequence meets the criteria set forth above, including the maintenance of biological protein, polypeptide or peptide activity where expression of a proteinaceous composition is concerned. The addition of terminal sequences particularly applies to nucleic acid sequences that may, for example, include various non-coding sequences flanking either of the 5′ and/or 3′ portions of the coding region or may include various internal sequences, i.e., introns, which are known to occur within genes.

The nucleic acids of the present invention encompass biologically functional equivalent sortase-transamidase proteins, polypeptides, or peptides or proteinaceous compositions comprising sorting signals. Such sequences may arise as a consequence of codon redundancy or functional equivalency that are known to occur naturally within nucleic acid sequences or the proteins, polypeptides or peptides thus encoded. Alternatively, functionally equivalent proteins, polypeptides or peptides may be created via the application of recombinant DNA technology, in which changes in the protein, polypeptide or peptide structure may be engineered, based on considerations of the properties of the amino acids being exchanged. Changes designed by man may be introduced, for example, through the application of site-directed mutagenesis techniques as discussed herein below, e.g., to introduce improvements or alterations to the antigenicity of the protein, polypeptide or peptide, or to test mutants in order to examine dehydrogenase protein, polypeptide or peptide activity at the molecular level.

Fusion proteins, polypeptides or peptides may be prepared, e.g., where the coding regions are aligned within the same expression unit with other proteins, polypeptides or peptides having desired functions. Non-limiting examples of such desired functions of expression sequences include purification or immunodetection purposes for the added expression sequences, e.g., proteinaceous compositions that may be purified by affinity chromatography or the enzyme labeling of coding regions, respectively.

Encompassed by the invention are nucleic acid sequences encoding relatively small peptides or fusion peptides, such as, for example, peptides of from about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, to about 100 amino acids in length, or more preferably, of from about 15 to about 30 amino acids in length. These can be applied with respect to SEQ ID NO:2 or SEQ ID NO:4.

As used herein an “organism” may be a prokaryote, eukaryote, virus and the like. As used herein the term “sequence” encompasses both the terms “nucleic acid” and “proteancecous” or “proteanaceous composition.” As used herein, the term “proteinaceous composition” encompasses the terms “protein”, “polypeptide” and “peptide.” As used herein “artificial sequence” refers to a sequence of a nucleic acid not, derived from sequence naturally occurring at a genetic locus, as well as the sequence of any proteins, polypeptides or peptides encoded by such a nucleic acid. A “synthetic sequence”, refers to a nucleic acid or proteinaceous composition produced by chemical synthesis in vitro, rather than enzymatic production in vitro (i.e., an “enzymatically produced” sequence) or biological production in vivo (i.e., a “biologically produced” sequence).

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

Other examples of expression systems include STRATAGENE®'s COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

It is contemplated that the proteins, polypeptides or peptides produced by the methods of the invention may be “overexpressed”, i.e., expressed in increased levels relative to its natural expression in cells. Such overexpression may be assessed by a variety of methods, including radio-labeling and/or protein purification. However, simple and direct methods are preferred, for example, those involving SDS/PAGE and protein staining or Western blotting, followed by quantitative analyses, such as densitometric scanning of the resultant gel or blot. A specific increase in the level of the recombinant protein, polypeptide or peptide in comparison to the level in natural cells is indicative of overexpression, as is a relative abundance of the specific protein, polypeptides or peptides in relation to the other proteins produced by the host cell and, e.g., visible on a gel.

In some embodiments, the expressed proteinaceous sequence forms an inclusion body in the host cell, the host cells are lysed, for example, by disruption in a cell homogenizer, washed and/or centrifuged to separate the dense inclusion bodies and cell membranes from the soluble cell components. This centrifugation can be performed under conditions whereby the dense inclusion bodies are selectively enriched by incorporation of sugars, such as sucrose, into the buffer and centrifugation at a selective speed. Inclusion bodies may be solubilized in solutions containing high concentrations of urea (e.g. 8M) or chaotropic agents such as guanidine hydrochloride in the presence of reducing agents, such as β-mercaptoethanol or DTT (dithiothreitol), and refolded into a more desirable conformation, as would be known to one of ordinary skill in the art.

The nucleotide and protein, polypeptide and peptide sequences for various genes have been previously disclosed, and may be found at computerized databases known to those of ordinary skill in the art. One such database is the National Center for Biotechnology Information's Genbank and GenPept databases (http://www.ncbi.nlm.nih.gov/). The coding regions for these known genes may be amplified and/or expressed using the techniques disclosed herein or by any technique that would be know to those of ordinary skill in the art. Additionally, peptide sequences may be synthesized by methods known to those of ordinary skill in the art, such as peptide synthesis using automated peptide synthesis machines, such as those available from Applied Biosystems (Foster City, Calif.).

### XII. LIPID COMPOSITIONS

In certain embodiments, the present invention concerns a novel composition comprising one or more lipids associated with at least one polypeptide. For example, in some embodiments of the invention there is a polypeptide comprising a sorting signal. A lipid is a substance that is characteristically insoluble in water and extractable with an organic solvent. Lipids include, for example, the substances comprising the fatty droplets that naturally occur in the cytoplasm as well as the class of compounds which are well known to those of skill in the art which contain long-chain aliphatic hydrocarbons and their derivatives, such as fatty acids, alcohols, amines, amino alcohols, and aldehydes. Of course, compounds other than those specifically described herein that are understood by one of skill in the art as lipids are also encompassed by the compositions and methods of the present invention.

A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof.

A. Lipid Types

A neutral fat may comprise a glycerol and a fatty acid. A typical glycerol is a three carbon alcohol. A fatty acid generally is a molecule comprising a carbon chain with an acidic moeity (e.g., carboxylic acid) at an end of the chain. The carbon chain may of a fatty acid may be of any length, however, it is preferred that the length of the carbon chain be of from about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, to about 30 or more carbon atoms, and any range derivable therein. However, a preferred range is from about 14 to about 24 carbon atoms in the chain portion of the fatty acid, with about 16 to about 18 carbon atoms being particularly preferred in certain embodiments. In certain embodiments the fatty acid carbon chain may comprise an odd number of carbon atoms, however, an even number of carbon atoms in the chain may be preferred in certain embodiments. A fatty acid comprising only single bonds in its carbon chain is called saturated, while a fatty acid comprising at least one double bond in its chain is called unsaturated.

Specific fatty acids include, but are not limited to, linoleic acid, oleic acid, palmitic acid, linolenic acid, stearic acid, lauric acid, myristic acid, arachidic acid, palmitoleic acid, arachidonic acid ricinoleic acid, tuberculosteric acid, lactobacillic acid. An acidic group of one or more fatty acids is covalently bonded to one or more hydroxyl groups of a glycerol. Thus, a monoglyceride comprises a glycerol and one fatty acid, a diglyceride comprises a glycerol and two fatty acids, and a triglyceride comprises a glycerol and three fatty acids.

A phospholipid generally comprises either glycerol or an sphingosine moiety, an ionic phosphate group to produce an amphipathic compound, and one or more fatty acids. Types of phospholipids include, for example, phophoglycerides, wherein a phosphate group is linked to the first carbon of glycerol of a diglyceride, and sphingophospholipids (e.g., sphingomyelin), wherein a phosphate group is esterified to a sphingosine amino alcohol. Another example of a sphingophospholipid is a sulfatide, which comprises an ionic sulfate group that makes the molecule amphipathic. A phopholipid may, of course, comprise further chemical groups, such as for example, an alcohol attached to the phosphate group. Examples of such alcohol groups include serine, ethanolamine, choline, glycerol and inositol. Thus, specific phosphoglycerides include a phosphatidyl serine, a phosphatidyl ethanolamine, a phosphatidyl choline, a phosphatidyl glycerol or a phosphotidyl inositol. Other phospholipids include a phosphatidic acid or a diacetyl phosphate. In one aspect, a phosphatidylcholine comprises a dioleoylphosphatidylcholine (a.k.a. cardiolipin), an egg phosphatidylcholine, a dipalmitoyl phosphalidycholine, a monomyristoyl phosphatidylcholine, a monopalmitoyl phosphatidylcholine, a monostearoyl phosphatidylcholine, a monooleoyl phosphatidylcholine, a dibutroyl phosphatidylcholine, a divaleroyl phosphatidylcholine, a dicaproyl phosphatidylcholine, a diheptanoyl phosphatidylcholine, a dicapryloyl phosphatidylcholine or a distearoyl phosphatidylcholine.

A glycolipid is related to a sphinogophospholipid, but comprises a carbohydrate group rather than a phosphate group attached to a primary hydroxyl group of the sphingosine. A type of glycolipid called a cerebroside comprises one sugar group (e.g., a glucose or galactose) attached to the primary hydroxyl group. Another example of a glycolipid is a ganglioside (e.g., a monosialoganglioside, a GM1), which comprises about 2, about 3, about 4, about 5, about 6, to about 7 or so sugar groups, that may be in a branched chain, attached to the primary hydroxyl group. In other embodiments, the glycolipid is a ceramide (e.g., lactosylceramide).

A steroid is a four-membered ring system derivative of a phenanthrene. Steroids often possess regulatory functions in cells, tissues and organisms, and include, for example, hormones and related compounds in the progestagen (e.g., progesterone), glucocoricoid (e.g., cortisol), mineralocorticoid (e.g., aldosterone), androgen (e.g., testosterone) and estrogen (e.g., estrone) families. Cholesterol is another example of a steroid, and generally serves structural rather than regulatory functions. Vitamin D is another example of a sterol, and is involved in calcium absorption from the intestine.

A terpene is a lipid comprising one or more five carbon isoprene groups. Terpenes have various biological functions, and include, for example, vitamin A, coenyzme Q and carotenoids (e.g., lycopene and β-carotene).

B. Charged and Neutral Lipid Compositions

In certain embodiments, a lipid component of a composition is uncharged or primarily uncharged. In one embodiment, a lipid component of a composition comprises one or more neutral lipids. In another aspect, a lipid component of a composition may be substantially free of anionic and cationic lipids, such as certain phospholipids (e.g., phosphatidyl choline) and cholesterol. In certain aspects, a lipid component of an uncharged or primarily uncharged lipid composition comprises about 95%, about 96%, about 97%, about 98%, about 99% or 100% lipids without a charge, substantially uncharged lipid(s), and/or a lipid mixture with equal numbers of positive and negative charges.

In other aspects, a lipid composition may be charged. For example, charged phospholipids may be used for preparing a lipid composition according to the present invention and can carry a net positive charge or a net negative charge. In a non-limiting example, diacetyl phosphate can be employed to confer a negative charge on the lipid composition, and stearylamine can be used to confer a positive charge on the lipid composition.

C. Making Lipids

Lipids can be obtained from natural sources, commercial sources or chemically synthesized, as would be known to one of ordinary skill in the art. For example, phospholipids can be from natural sources, such as egg or soybean phosphatidylcholine, brain phosphatidic acid, brain or plant phosphatidylinositol, heart cardiolipin and plant or bacterial phosphatidylethanolamine. In another example, lipids suitable for use according to the present invention can be obtained from commercial sources. For example, dimyristyl phosphatidylcholine (“DMPC”) can be obtained from Sigma Chemical Co., dicetyl phosphate (“DCP”) is obtained from K & K Laboratories (Plainview, N.Y.); cholesterol (“Chol”) is obtained from Calbiochem-Behring; dimyristyl phosphatidylglycerol (“DMPG”) and other lipids may be obtained from Avanti Polar Lipids, Inc. (Birmingham, Ala.). In certain embodiments, stock solutions of lipids in chloroform or chloroform/methanol can be stored at about −20° C. Preferably, chloroform is used as the only solvent since it is more readily evaporated than methanol.

D. Lipid Composition Structures

In an embodiment of the invention, a polypeptide, such as one having a sorting signal, may be associated with a lipid. A chimeric polypeptide associated with a lipid may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure. A lipid or lipid/chimeric polypeptide associated composition of the present invention is not limited to any particular structure. For example, they may also simply be interspersed in a solution, possibly forming aggregates which are not uniform in either size or shape. In another example, they may be present in a bilayer structure, as micelles, or with a “collapsed” structure. In another non-limiting example, a lipofectamine (Gibco BRL)-chimeric polypeptide or Superfect (Qiagen)-chimeric polypeptide complex is also contemplated.

In certain embodiments, a lipid composition may comprise about 1%, about 2%, about 3%, about 4% about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, or any range derivable therein, of a particular lipid, lipid type or non-lipid component such as a drug, protein, sugar, nucleic acids or other material disclosed herein or as would be known to one of skill in the art. In a non-limiting example, a lipid composition may comprise about 10% to about 20% neutral lipids, and about 33% to about 34% of a cerebroside, and about 1% cholesterol. In another non-limiting example, a liposome may comprise about 4% to about 12% terpenes, wherein about 1% of the micelle is specifically lycopene, leaving about 3% to about 11% of the liposome as comprising other terpenes; and about 10% to about 35% phosphatidyl choline, and about 1% of a drug. Thus, it is contemplated that lipid compositions of the present invention may comprise any of the lipids, lipid types or other components in any combination or percentage range.

1. Emulsions

A lipid may be comprised in an emulsion. A lipid emulsion is a substantially permanent heterogenous liquid mixture of two or more liquids that do not normally dissolve in each other, by mechanical agitation or by small amounts of additional substances known as emulsifiers. Methods for preparing lipid emulsions and adding additional components are well known in the art (e.g., Modern Pharmaceutics, 1990, incorporated herein by reference).

For example, one or more lipids are added to ethanol or chloroform or any other suitable organic solvent and agitated by hand or mechanical techniques. The solvent is then evaporated from the mixture leaving a dried glaze of lipid. The lipids are resuspended in aqueous media, such as phosphate buffered saline, resulting in an emulsion. To achieve a more homogeneous size distribution of the emulsified lipids, the mixture may be sonicated using conventional sonication techniques, further emulsified using microfluidization (using, for example, a Microfluidizer, Newton, Mass.), and/or extruded under high pressure (such as, for example, 600 psi) using an Extruder Device (Lipex Biomembranes, Vancouver, Canada).

2. Micelles

A lipid may be comprised in a micelle. A micelle is a cluster or aggregate of lipid compounds, generally in the form of a lipid monolayer, and may be prepared using any micelle producing protocol known to those of skill in the art (e.g., Canfield et al., 1990; El-Gorab et al, 1973; Colloidal Surfactant, 1963; and Catalysis in Micellar and Macromolecular Systems, 1975, each incorporated herein by reference). For example, one or more lipids are typically made into a suspension in an organic solvent, the solvent is evaporated, the lipid is resuspended in an aqueous medium, sonicated and then centrifuged.

3. Liposomes

In particular embodiments, a lipid comprises a liposome. A “liposome” is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition.

A multilamellar liposome has multiple lipid layers separated by aqueous medium. They form spontaneously when lipids comprising phospholipids are suspended in an excess of aqueous solution. The lipid components undergo self-rearrangement before the formation of closed structures and entrap water and dissolved solutes between the lipid bilayers (Ghosh and Bachhawat, 1991). Lipophilic molecules or molecules with lipophilic regions may also dissolve in or associate with the lipid bilayer.

In certain less preferred embodiments, phospholipids from natural sources, such as egg or soybean phosphatidylcholine, brain phosphatidic acid, brain or plant phosphatidylinositol, heart cardiolipin and plant or bacterial phosphatidylethanolamine are preferably not used as the primary phosphatide, i.e., constituting 50% or more of the total phosphatide composition or a liposome, because of the instability and leakiness of the resulting liposomes.

In particular embodiments, a lipid and/or polypeptide may be, for example, encapsulated in the aqueous interior of a liposome, interspersed within the lipid bilayer of a liposome, attached to a liposome via a linking molecule that is associated with both the liposome and the polypeptide, entrapped in a liposome, complexed with a liposome, etc.

### XIII. PHARMACEUTICAL PREPARATIONS

A. Effective Dosages

The proteins of the invention will generally be used in an amount effective to achieve the intended purpose. For use to treat or prevent a disease condition, the proteins of the invention, or pharmaceutical compositions thereof, are administered or applied in a therapeutically effective amount. A therapeutically effective amount is an amount effective to ameliorate or prevent the symptoms, or prolong the survival of, the patient being treated. Determination of a therapeutically effective amount is well within the capabilities of those skilled in the art, especially in light of the detailed disclosure provided herein.

For systemic administration, a therapeutically effective dose can be estimated initially from in vitro assays. For example, a dose can be formulated in animal models to achieve a circulating concentration range that includes the IC₅ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the proteins which are sufficient to maintain therapeutic effect. Usual patient dosages for administration by injection range from about 0.1 to 5 mg/kg/day, preferably from about 0.5 to 1 mg/kg/day. Therapeutically effective serum levels may be achieved by administering multiple doses each day.

In cases of local administration or selective uptake, the effective local concentration of the proteins may not be related to plasma concentration. One having skill in the art will be able to optimize therapeutically effective local dosages without undue experimentation.

The amount of protein administered will, of course, be dependent on the subject being treated, on the subject's weight, the severity of the affliction, the manner of administration and the judgment of the prescribing physician.

The therapy may be repeated intermittently while symptoms detectable or even when they are not detectable. The therapy may be provided alone or in combination with other drugs. In the case of bacterial infection, the drugs that may be used in combination with compositions of the invention include, but are not limited to, antibiotics.

Pharmaceutical compositions of the present invention comprise an effective amount of one or more candidate substance, therapeutic agents, a polypeptide of interest or additional agent dissolved or dispersed in a pharmaceutically acceptable carrier. It is an aspect of the invention that candidate substance, which is shown to modulate a sortase-transamidase such as SrtA or SrtB, be prepared for pharmaceutical administration. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one polypeptide of interest, candidate substance or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The sortase-transamidase or candidate substance may comprise different types of carriers depending on whether it is to be administered in solid, liquid or aerosol form, and whether it need to be sterile for such routes of administration as injection. The present invention can be administered intraocularly, intravenously, intradermally, intraarterially, intraperitoneally, intracranially, topically, intramuscularly, intraperitoneally, subcutaneously, intravesicularlly, mucosally, orally, topically, locally, inhalation (e.g. aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The sortase-transamidase, polypeptide of interest, or candidate substance may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present invention. Such compositions are generally designed to be compatible with the target tissue type. In a non-limiting example, the sortase-transamidase, the polypeptide of interest, or candidate substance is prepared for administration by eye drops. The pupil may be dilated prior to administration of the candidate substance.

In certain embodiments the sortase-transamidase, the polypeptide of interest, or candidate substance is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments, the composition may comprise an ophthalmic solution, an ophthalmic suspension, an ophthalmic ointment, an ocular insert or an intraocular solution. Other modes of administration to the eye include packs, intracameral injections which are made directly into the anterior chamber, iontophoresis wherein an eyecup bearing an electrode keeps the therapeutic agent in contact with the cornea, subconjunctival injections for the introduction of therapeutic agents that do not penetrate into the anterior segment or penetrate too slowly and retrobulbar injections for delivery of therapeutic agents predominantly into the posterior section of the globe.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof, an excipient, such as, for example, dicalcium phosphate, mannitol, lactose, starch, magnesium stearate, sodium saccharine, cellulose, magnesium carbonate or combinations thereof, a disintegrating agent, such as, for example, corn starch, potato starch, alginic acid or combinations thereof, a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

B. In Vitro, Ex Vivo, In Vivo Administration

As used herein, the term in vitro administration refers to manipulations performed on cells removed from an animal, including, but not limited to, cells in culture. The term ex vivo administration refers to cells which have been manipulated in vitro, and are subsequently administered to a living animal. The term in vivo administration includes all manipulations performed on cells within an animal.

In certain aspects of the present invention, the compositions may be administered either in vitro, ex vivo, or in vivo. In certain in vitro embodiments, a Gram-positive bacteria is incubated with a sortase-transamidase and a polypeptide comprising a sorting signal and an antigen against which an immune response is desired. Examples of antigens include bacterial and viral proteins, but also tumor-associated antigens and other disease-related antigens. The resultant bacteria displaying the antigen can then be implemented as a vaccine in vitro analysis, or alternatively for in vivo administration.

U.S. Pat. Nos. 6,150,170 and 6,022,728, incorporated herein by reference, discloses methods for making bacterial vaccines and for use in therapeutic applications.

In vivo administration of the compositions of the present invention are also contemplated. Examples include, but are not limited to, transduction of bladder epithelium by administration of the transducing compositions of the present invention through intravesicle catheterization into the bladder (Bass, 1995), and transduction of liver cells by infusion of appropriate transducing compositions through the portal vein via a catheter (Bao, 1996). Additional examples include direct injection of tumors with the instant transducing compositions, and either intranasal or intratracheal (Dong, 1996) instillation of transducing compositions to effect transduction of lung cells.

C. Vaccines

The present invention includes methods for preventing or treating a disease or infection using a vaccine based on a Gram-positive bacterium of the invention based on the ability of the bacterium to display any desired antigen on its surface. The use of attenuated bacteria is contemplated, wherein the attenuated bacteria has reduced pathogenicity, reduced virulence, an inability to replicate, reduced immunogencity against the bacteria itself, or any combination thereof. As such, the invention contemplates vaccines for use in both active and passive immunization embodiments. Immunogenic compositions, proposed to be suitable for use as a vaccine, may be prepared most readily directly from polypeptides prepared in a manner disclosed herein. Preferably the antigenic material is extensively dialyzed to remove undesired small molecular weight molecules and/or lyophilized for more ready formulation into a desired vehicle.

Alternatively, other viable and important options for a vaccine involve introducing the polypeptide sequences as nucleic acids, either as direct DNA vaccines or recombinant vaccinia virus-based polyepitope vaccine. In this regard, recent reports described construction of recombinant vaccinia viruses expressing either 10 contiguous minimal CTL epitopes (Thomson, 1996) or a combination of B cell, CTL, and TH epitopes from several microbes (An, 1997), and successful use of such constructs to immunize mice for priming protective immune responses. Thus, there is ample evidence in the literature for successful utilization of peptides, peptide-pulsed APCs, and peptide-encoding constructs for efficient in vivo priming of protective immune responses, in particular, CMI.

The preparation of other vaccines, for example that contain HIV peptide sequences as active ingredients, is generally well understood in the art, as exemplified by U.S. Pat. Nos. 4,608,251; 4,601,903; 4,599,231; 4,599,230; 4,596,792; and 4,578,770, all incorporated herein by reference. Typically, such vaccines are prepared as injectables either as liquid solutions or suspensions: solid forms suitable for solution in or suspension in liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, or the like and combinations thereof. In addition, if desired, the vaccine may contain minor amounts of auxiliary substances such as wetting or emulsifying agents, pH buffering agents, or adjuvants that enhance the effectiveness of the vaccines.

Vaccines may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkalene glycols or triglycerides: such suppositories may be formed from mixtures containing the active ingredient in the range of about 0.5% to about 10%, preferably about 1% to about 2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, preferably about 25% to about 70%.

The polypeptides and Gram-positive bacteria of the present invention may be formulated into the vaccine as neutral or salt forms. Pharmaceutically-acceptable salts include the acid addition salts (formed with the free amino groups of the peptide) and those that are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The vaccines are administered in a manner compatible with the dosage formulation, and in such amount as will be therapeutically effective and immunogenic. The quantity to be administered depends on the subject to be treated, including, e.g., the capacity of the individual's immune system to synthesize antibodies and the degree of protection desired. Precise amounts of active ingredient required to be administered depend on the judgment of the practitioner. However, suitable dosage ranges are of the order of several hundred micrograms active ingredient per vaccination. Suitable regimes for initial administration and booster shots are also variable, but are typified by an initial administration followed by subsequent inoculations or other administrations.

The manner of application may be varied widely. Any of the conventional methods for administration of a vaccine are applicable. These are believed to include oral application on a solid physiologically acceptable base or in a physiologically acceptable dispersion, parenterally, by injection or the like. The dosage of the vaccine will depend on the route of administration and will vary according to the size of the host.

Various methods of achieving adjuvant effect for the vaccine includes use of agents such as aluminum hydroxide or phosphate (alum), commonly used as about 0.05 to about 0.1% solution in phosphate buffered saline, admixture with synthetic polymers of sugars (Carbopol®) used as an about 0.25% solution, aggregation of the protein in the vaccine by heat treatment with temperatures ranging between about 70° to about 101° C. for a 30-second to 2-minute period, respectively. Aggregation by reactivating with pepsin-treated (Fab) antibodies to albumin, mixture with bacterial cells such as C. parvum or endotoxins or lipopolysaccharide components of Gram-negative bacteria, emulsion in physiologically acceptable oil vehicles such as mannide mono-oleate (Aracel A), or emulsion with a 20% solution of a perfluorocarbon (Fluosol-DA®) used as a block substitute may also be employed.

In many instances, it will be desirable to have multiple administrations of the vaccine, usually not exceeding six vaccinations, more usually not exceeding four vaccinations and preferably one or more, usually at least about three vaccinations. The vaccinations will normally be at from two to twelve week intervals, more usually from three to five week intervals. Periodic boosters at intervals of 1-5 years, usually three years, will be desirable to maintain protective levels of the antibodies. The course of the immunization may be followed by assays for antibodies for the supernatant antigens. The assays may be performed by labeling with conventional labels, such as radionuclides, enzymes, fluorescents, and the like. These techniques are well known and may be found in a wide variety of patents, such as U.S. Pat. Nos. 3,791,932; 4,174,384 and 3,949,064, as illustrative of these types of assays.

In particular embodiments, prolonged absorption of an injectable composition can be brought about by the use in the compositions of agents delaying absorption, such as, for example, aluminum monostearate, gelatin or combinations thereof.

D. Toxicity

Preferably, a therapeutically effective dose of the chimeric proteins described herein will provide therapeutic benefit without causing substantial toxicity.

Toxicity of the proteins described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. Proteins which exhibit high therapeutic indices are preferred. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1, p. 1).

### XIV. COMBINATIONAL THERAPIES

Therapies for bacterial infection, including infection from Gram-positive bacteria, are known to one of skill in the art, may be used in combination with the therapeutic agents obtained from polypeptides of the invention. Thus, in order to increase the effectiveness of the therapy using a therapeutic agent, or expression construct coding therefor, it may be desirable to combine these compositions with other agents effective in the treatment of bacterial infections such as but not limited to those described below. For example, one can use one can use the therapeutic agent-based therapy of the invention in conjunction with surgery and/or antiobiotics, and/or other therapeutic methods.

The other therapy may precede or follow the therapeutic agent-based therapy by intervals ranging from minutes to days to weeks. In embodiments where the other macular or retinal degeneration therapy and the therapeutic agent-based therapy are administered together, one would generally ensure that a significant period of time did not expire between the time of each delivery. In such instances, it is contemplated that one would administer to a patient both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either the other antibacterial therapy and the therapeutic agent-based therapy will be required to treat the infection. Various combinations may be employed, where the other macular or retinal degeneration therapy is “A” and the therapeutic agent-based therapy treatment is “B”, as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations also are contemplated. The exact dosages and regimens can be suitable altered by those of ordinary skill in the art.

Antiobiotics often used for Gram-positive infections is shown in the table below (TABLE 4). Any of the antiobiotics shown, or a combination thereof, may be implemented with a therapeutic or preventative agent of the invention.

TABLE 4

| | | | Spectrum | |
|---|---|---|---|---|
| Chemical class | Examples | Biological source | (effective against) | Mode of action |
| Beta-lactams | Penicillin G, | Penicillium notatum | Gram-positive | Inhibits steps in cell |
| (penicillins and | Cephalothin | and Cephalosporium | bacteria | wall (peptidoglycan) |
| cephalosporins) | Methicillin | species | | synthesis and murein |
| | Cefaclor | | | assembly |
| Semisynthetic | Ampicillin, | | Gram-positive and | Inhibits steps in cell |
| penicillin and | Amoxycillin | | Gram-negative | wall (peptidoglycan) |
| cephalosporins | Cefaclor | | bacteria | synthesis and murein |
| | Cefazolin | | | assembly |
| | Cefotaxime | | | |
| | Nafcillin | | | |
| | Oxacillin | | | |
| Clavulanic Acid | Clavamox is | Streptomyces | Gram-positive and | Suicide inhibitor of |
| | clavulanic acid | clavuligerus | Gram-negative | beta-lactamases |
| | plus amoxycillin | | bacteria | |
| Novobiocin | Novobiocin | Streptomyces nivens | Gram-positive | |
| | | | bacteria | |
| Monobactams | Aztreonam | Chromobacter | Gram-positive and | Inhibits steps in cell |
| | | violaceum | Gram-negative | wall (peptidoglycan) |
| | | | bacteria | synthesis and murein |
| | | | | assembly |
| Carboxypenems | Imipenem | Streptomyces | Gram-positive and | Inhibits steps in cell |
| | | cattleya | Gram-negative | wall (peptidoglycan) |
| | | | bacteria | synthesis and murein |
| | | | | assembly |
| Aminoglycosides | Streptomycin | Streptomyces | Gram-positive and | Inhibit translation |
| | | griseus | Gram-negative | (protein synthesis) |
| | | | bacteria | |
| | Gentamicin | Micromonospora | Gram-positive and | Inhibit translation |
| | | species | Gram-negative | (protein synthesis) |
| | | | bacteria esp. | |
| | | | Pseudomonas | |
| Glycopeptides | Vancomycin | Streptomyces | Gram-positive | Inhibits steps in |
| | | orientales | bacteria, esp. | murein |
| | | | Staphylococcus | (peptidoglycan) |
| | | | aureus | biosynthesis and |
| | | | | assembly |
| Lincomycins | Clindamycin | Streptomyces | Gram-positive and | Inhibits translation |
| | | lincolnensis | Gram-negative | (protein synthesis) |
| | | | bacteria esp. | |
| | | | anaerobic | |
| | | | Bacteroides | |
| Macrolides | Erythromycin | Streptomyces | Gram-positive | Inhibits translation |
| | Azithromycin | erythreus | bacteria, Gram- | (protein synthesis) |
| | Oleandomycin | | negative bacteria not | |
| | | | enterics, | |
| | | | Neisseria, Legionella, | |
| | | | Mycoplasma | |
| Oxazolidines | Linezolid | | Gram positive | |
| | | | bacteria | |
| Polypeptides | Polymyxin | Bacillus polymyxa | Gram-negative | Damages cytoplasmic |
| | | | bacteria | membranes |
| | Bacitracin | Bacillus subtilis | Gram-positive | Inhibits steps in |
| | | | bacteria | murein |
| | | | | (peptidoglycan) |
| | | | | biosynthesis and |
| | | | | assembly |
| Isoniazids | Isoniazid | | Gram-positive | Inhibits mycolic acid |
| | | | bacteria | synthesis, analog of |
| | | | | pyridoxine. |
| Rifamycins | Rifampicin | Streptomyces | Gram-positive and | Inhibits transcription |
| | | mediterranei | Gram-negative | (eubacterial RNA |
| | | | bacteria, | polymerase) |
| | | | Mycobacterium | |
| | | | tuberculosis | |
| Tetracyclines | Tetracycline | Streptomyces | Gram-positive and | Inhibit translation |
| | Chlortetracycline | species | Gram-negative | (protein synthesis) |
| | | | bacteria, Rickettsias | |
| Semisynthetic | Doxycycline | | Gram-positive and | Inhibit translation |
| tetracycline | | | Gram-negative | (protein synthesis) |
| | | | bacteria, Rickettsias | |
| | | | Ehrlichia, Borellia | |
| Chloramphenicol | Chloramphenicol | Streptomyces | Gram-positive and | Inhibits translation |
| | | venezuelae | Gram-negative | (protein synthesis) |
| | | | bacteria | |

E. Kits

All the essential materials and/or reagents required for detecting SEQ ID NO. 2, SEQ ID NO. 4 in a sample may be assembled together in a kit. This generally will comprise a probe or primers designed to hybridize specifically to individual nucleic acids of interest in the practice of the present invention, including SEQ ID NO. 1, SEQ ID NO. 3. Also included may be enzymes suitable for amplifying nucleic acids, including various polymerases (reverse transcriptase, Taq, etc.), deoxynucleotides and buffers to provide the necessary reaction mixture for amplification. Such kits may also include enzymes and other reagents suitable for detection of specific nucleic acids or amplification products. Such kits generally will comprise, in suitable means, distinct containers for each individual reagent or enzyme as well as for each probe or primer pair.

### XV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Examples 1-4 are copied from the U.S. Patent Application of Olaf Schneewind filed in 2001 and entitled “Identification of Sortase Gene,” which is specifically incorporated by reference in its entirety herein.

### Example 1

### Identification of a Staphylococcal Mutant Defective in Cell Wall Sorting Generation of Temperature Sensitive (ts) Mutants Through Chemical Mutagenesis

Cell wall sorting mutants were created and isolated from a population of conditional lethal mutants of S. aureus strain OS2. Staphylococci were mutagenized with nitrosoguanidine and colonies were formed by plating at 30° C. Bacteria were streaked and incubated at 30° C. and 42° C. to identify mutants that are temperature sensitive for growth (ts). A collection of one thousand is mutants was transformed with pSEB-SPA₄₉₀₋₅₂₄ (Schneewind et al., 1993), specifying a reporter protein for measurements of surface protein anchoring. The SEB-SPA₄₉₀₋₅₂₄ precursor (P1) is exported from the cytoplasm and its NH₂ terminal leader peptide removed to generate the P2 intermediate. The P2 precursor is the substrate for sortase, which cleaves the polypeptide between the threonine and the glycine of the LPXTG motif and generates mature, anchored surface protein (M). When analyzed by labeling wild-type staphylococci with [³⁵S]methionine for 5 min, cleavage of P1 precursor is faster than that of the P2 species, yielding a ratio of P1 (5%), P2 (19%), and M (76%) concentration. This assay was employed to screen one thousand ts mutants and two strains were identified that accumulated P2 precursor at 47% (SM317) and 26% (SM329), respectively. To examine the sorting reaction further, mutant and wild-type staphylococci were subjected to pulsechase analysis. S. aureus OS2 (wild-type) cleaved and anchored the P1 precursor within 2 min. The sorting reaction in strain SM317 was severely reduced as cleavage and cell wall anchoring of pulse-labeled P2 required more than 10 min. Strain SM329 displayed only a weak defect and P2 processing -required 3 min. When examined by pulse-labeling staphylococci grown in minimal medium, SM329 displayed a much more severe defect in cell wall sorting.

Anchor Structure of Surface Proteins in the Mutant Strain SM317

To examine whether the mutant strains SM317 and SM329 are defective in the synthesis of bacterial cell wall, two tests were performed. Lysostaphin is a bacteriolytic enzyme that cuts the pentaglycine crossbridges of the staphylococcal cell wall predominantly at the central glycine residue (Schindler et al., 1992). As reported previously, fem mutants display resistance to this bacteriocin and grow even in the presence of large amounts of lysostaphin (Kopp et al., 1996). Strains SM317 and SM329 were sensitive to lysostaphin at concentrations that also inhibited growth of wild-type staphylococci, indicating that the sorting defect in SM317 is not caused by a mutationally altered cell wall crossbridge. To measure bacterial cell wall synthesis, staphylococci were grown in minimal medium and labeled with [³H]lysine and [³H]leucine (Boothby et al., 1971). As lysine, but not leucine, is a component of the bacterial cell wall, the ratio of [³H]lysine/[³H]leucine incorporation into acid precipitable and protease resistant murein polymer is a measure for cell wall synthesis (Boothby et al., 1971). Wild-type staphylococci displayed a ratio of 30, while the addition of vancomycin to the culture medium reduced the ratio of incorporated lysine/leucine to 1.5 (20-fold inhibition). Strains SM317 and SM329 displayed a ratio of 18 and 19 (1.6 fold less than wild-type cells), suggesting that the accumulation of P2 precursor in the mutant SM317 is not caused by a defect in cell wall synthesis.

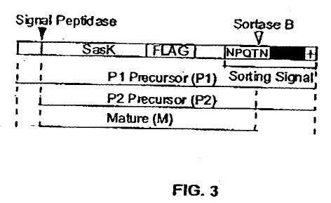

The cell wall anchor structure of surface protein in strain SM317 was determined. Plasmid pHTT4 specifying the reporter protein SEB-MH₆-CWS was transformed into S. aureus SM317 (Ton-That et al., 1997). The staphylococcal cell wall was purified and digested with mutanolysin, a muramidase that hydrolyzes the glycan strands (Yokogawa et al., 1974). Mutanolysin-released surface protein was purified by chromatography on Ni-NTA and cleaved at methionine residues with cyanogen bromide (Ton-That et al., 1997). COOH-terminal peptides bearing cell wall anchor structures were purified by a second affinity chromatography step and analyzed by MALDI-MS. A series of ion signals with regularly spaced mass increments was revealed, measurements that are consistent with one, two, three, four, five and six peptidoglycan subunits linked to the COON-terminal threonine of surface protein. Ion signals of muanolysin-solubilized anchor peptides were explained as H₆AQALPET-GIy5 linked to cell wall tetrapeptide (predicted mass 2235; observed 2236), pentapeptide (predicted mass 2306; observed 2306), N,06-diacetyIMurNac-GicNac tetrapeptide (predicted mass 2755, observed 2756), N,06-diacetylMurNac-GicNac pentapeptide (predicted mass 2826, observed 2826), murein-tetrapeptide-murein-pentapeptide (predicted mass 3991, observed 3995), (murein-tetrapeptide)₂-murein-pentapeptide (predicted mass 5194; observed 5196), (murein-tetrapeptide)₄ (predicted mass 6285 observed 6285), (murein-tetrapeptide)₄-murein-pentapeptide (predicted mass 7581; observed 7583), (murein-tetrapeptide)5-murein-pentapeptide (predicted mass 8783; observed 8784). If surface protein is tethered to cross-linked peptidoglycan of strain SM317, digestion of muramidase-solubilized anchor peptides with f11 hydrolase should produce anchor peptide linked to murein tetrapeptide and disaccharide-tetrapeptide (Ton-That et al., 1999). This was tested and the doubly digested anchor peptides generated ion signals at m/z 2236 [L-Ala-D-iGln-L-Lys(NH₂-H6_{A}QALPET-GLy₅)-D-Ala, predicted mass 2235], 2714 [MurNac(L-Ala-D-iGln-L-Lys(NH₂-H₆AQALPE-Gly₅)-D-AIa)-GlcNac, predicted mass 2713) and 2756 [06-acetyl-MurNac(L-Ala-D-iGln-L-Lys(NH₂-H6_{A}QALPET-Gly₅)-D-Ala)-GIcNac, predicted mass 2756] (FIG. 3C). Thus, surface proteins of S. aureus SM317 are tethered to cross-linked peptidoglycan in a manner that is indistinguishable from the anchor structure of polypeptides in wild-type staphylococci (Navarre et al., 1998). These results suggest that the accumulation of P2 precursor in strain SM317 is likely caused by a defect in sortase.

Screening for the Sortase Gene

Over-expression of sortase from a multi-copy plasmid should reduce the concentration of P2 in both wild-type and mutant staphylococci. A plasmid library of two thousand 3-5 kb random S. aureus OS2 chromosomal DNA insertions was screened for sequences that caused a reduction in the concentration of P2 precursor in strain SM317. Two plasmids pGL1631 and pGL1834, answered this screen. Transformation with pGL1834 reduced the P2 concentration in strain SM317 from 44% to 9%, in strain SM329 from 26% to 12%, and in wild-type S. aureus OS2 from 17% to 8%. When measured by pulse-chase analysis, S. aureus OS2 (pGL1834) displayed a rapidly increased processing of P2 precursors, a phenotype that was also observed in strains SM317 and SM329. DNA sequencing revealed that pGL1631 and pGL1834 contained staphylococcal chromosomal DNA insertions with identical overlapping sequences. The DNA sequence sufficient to promote a reduction in P2 concentration was mapped to a gene which was named srtA (surface protein sorting A).

The srtA gene

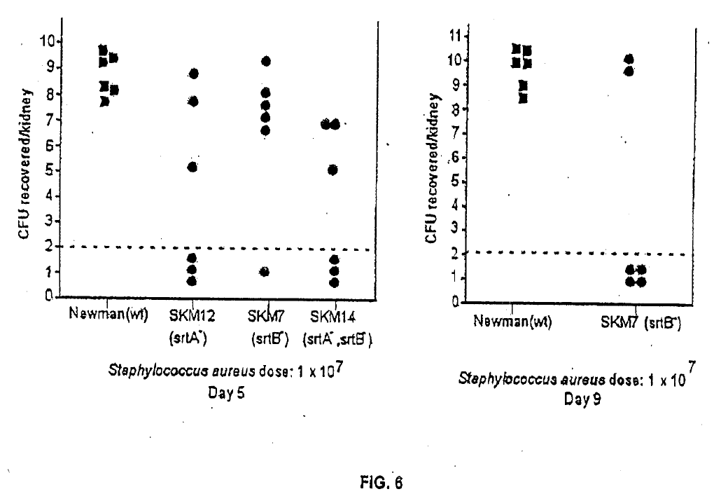

The srtA gene (SEQ ID NO:1) specifies a polypeptide chain of 206 amino acids (FIG. 6; SEQ ID NO:2). A sequence of 18 hydrophobic amino acids near the NH₂-terminus suggests the presence of a signal peptide/membrane anchor sequence. This feature is consistent with the notion that cell wall anchoring occurs on the cell surface, after polypeptide substrates bearing an LPXTG motif have been translocated across the cytoplasmic membrane. Another property of the srtA gene consistent with its function as sortase is the presence of codon 184 specifying cysteine. As the cell wall sorting reaction is sensitive to methanethiosulfonate, a reagent that forms disulfide with sulfhydryl (Smith et al., 1975), the presence of a cysteine must be a conserved feature of sortase homologues.

Many, if not all, Gram-positive pathogens display proteins on their surface via a sorting signal mediated mechanism (Navarre, 1999). Thus, if the srtA gene specifies sortase, homologous genes should be found in the genomes of other Gram-positive pathogens. Chromosomal DNA sequences of Enterococcus faecalis, Staphylococcus aureus, Streptococcus pyogenes, Streptococcus pneumoniae, and Streptococcus mutans were searched and the presence of srtA genes revealed. Database searches also identified sequences homologous to srtA in Bacillus subtilis and Actinomyces naeslundii. All srtA homologues displayed absolute conservation of the cysteine and striking conservation of the peptide sequences surrounding it. S. pneumoniae harbors more than one srtA homologue, which we have named srtB and srtC, respectively. The srtA like genes of E. faecalis and A. naeslundii are immediately adjacent to structural genes specifying surface proteins with a COOH-terminal sorting signal. The presence of a srtA homologue in the chromosome of B. subtilis is surprising as LPXTG motif containing sorting signals have not yet been identified in this organism. One of the srtA homologues in A. naeslundii, previously designated orf365, has been mutated. which abolished fimbrial assembly of mutant Actinomyces (Yeung, et al., 1998). Actinomyces fimbriae are composed of protein subunits bearing LPXTG motifs (Yeung et al., 1990), however the mechanism of fimbrial assembly (polymerization) is not yet understood.

The srtA Gene in Strain SM317

To examine whether the defect in cell wall sorting of S. aureus SM317 is caused by a mutation in the srtA gene, corresponding sequences were PCR amplified from the chromosomal DNA of S. aureus OS2 and SM317. When cloned into a multicopy vector and transformed into S. aureus SM317, the srtA gene amplified from wild-type staphylococci reduced the P2 concentration from 44% to 12%, while the same gene amplified from the chromosomal DNA of S. aureus SM317 did not reduce the P2 concentration of the parent strain. Thus, the srtA gene is defective in strain SM317 and DNA sequencing identified mutations in codons 35 and 180. The expression of wild-type srtA in SM317 in the is phenotype of the mutant strain was examined. Multi-copy expression of srtA (pGL1894) allowed growth of SM317 at 42° C. albeit at a rate that was less than that observed for wild-type staphylococci. This result suggests that the conditional lethal phenotype of S. aureus SM317 is not only caused a mutation in the srtA gene. Expression of plasmid encoded wild-type srtA did not alter the is growth phenotype of S. aureus SM329.

Sortase and the Cell Wall Sorting Reaction

The srtA gene was isolated as a multi-copy suppressor of P2 precursor accumulation, a scheme that should only be answered by the gene for sortase. Only one gene (srtA) from a library of two thousand plasmid transformants bearing random 35 kb chromosomal DNA insertions was observed this screen. Additional observations show SrtA protein catalyzes the in vitro transpeptidation of substrates bearing an LPXTG motif, thereby demonstrating that SrtA displays sortase activity. Purified SrtA protein can be used for the screening of compounds that inhibit sortase. Such compounds may be useful for the treatment of human infections caused by Gram-positive bacteria.

Materials and Methods

Mutagenesis of S. aureus Strain OS2

Staphylococci (1×1012 cfu) were treated with 0.2 mg/ml N-methyl-N′-nitro-N-nitrosoguanidine for 45 min at 30° C. and mutagenesis was quenched by the addition of 2 volumes of 100 mM sodium phosphate, pH 7.0. Approximately 80% of the mutagenized population was killed and the mutational frequency of rifampicin resistant rpo8 mutations was increased to 1.2×10⁻⁴. Temperature sensitive mutants were selected by growing the mutagenized population in tryptic soy broth at 42° C. and treating with 8 μg/ml penicillin G for two hours, a selection that was repeated twice. Colonies were formed at 30° C., streaked on tryptic soy agar and examined for growth at 42° C.

Transformation of Competent Cells

Staphylococci were grown in tryptic soy broth supplemented with chloramphenicol (10 mg/ml) or tetracycline (2 mg/ml) at 30° C. until OD₆₆₀ 0.6. Cells were incubated at 42° C. for 20 min, sedimented by centrifugation at 15,000×g for 3 min and washed with 1 ml of prewarmed minimal medium [Schneewind et al., 1992]. Staphylococci were labeled with 50 mCi of [³⁵S]-Promix (Amersham) for 5 min and surface protein processing quenched by the addition of 75 ml 100% TCA. The TCA precipitates were collected by centrifugation, washed in acetone and dried under vacuum. Samples were suspended in 1 ml of 0.5 M Tris-HCl, pH 7.0 and staphylococcal peptidoglycan was digested by adding 50 ml 2 mg/ml lysostaphin (AMBI Pharmaceuticals) for 1 h at 37° C. Proteins were again precipitated with TCA, washed with acetone and, after immunoprecipitation with a-SEB, were analyzed by 14% SDS-PAGE and Phosphorlmager.

Pulse-Chase Screen of Mutants

Staphylococci were grown as described above and 5 ml were labeled with 500 mCi of [³⁵S]-Promix (Amersham) for 45 secs. Incorporation of radioactivity was quenched by adding 50 ml chase (100 mg/ml casamino acids, 20 mg/ml methionine and cysteine). At timed intervals after the addition of the chase, 1 ml aliquots were removed and protein was precipitated by the addition of 75 ml 100% TCA. Sample preparation followed the same steps as described above.

DNA Sequencing

The DNA insertions pf pGL1631 and 1834 were mapped and sequenced by synthesizing of oligonucleotide primers that annealed to sequenced template DNA 500 nucleotides apart. The primers for the amplification of srtA from the chromosomal DNA of S. aureus strains OS2 and SM317 were 5′-AAAAA-3′ and 5′-TTTTTT-3′.

### Example 2

### Inhibitors of Cell Wall Sorting

To study the effects of antibiotic cell wall synthesis inhibitors interfered with the anchoring of surface proteins, the activity of several inhibitors were examined in a Gram-pcsitive bacteria sorting assay. A search for chemical inhibitors of the sorting reaction identified methanethiosulfonates and p-hydroxymercuribenzoic acid. Thus, sortase, the enzyme proposed to cleave surface proteins at the LPXTG motif, appears to be a sulfhydryl containing enzyme that utilizes peptidoglycan precursors but not assembled cell wall as a substrate for the anchoring of surface protein.

In order to identify compounds that interfere with the anchoring of surface proteins a reporter protein Seb-Spa₄₉₀₋₅₂₄ which, when expressed in S. aureus OS2 cells, is synthesized as a precursor in the cytoplasm and initiated into the secretory pathway by an NH₂-terminal leader peptide (P1 precursor) was utilized (Schneewind et al., 1993). After signal peptide cleavage, the P2 precursor bearing a COOH-terminal sorting signal serves as a substrate for sortase, an enzyme that cleaves between the threonine and the glycine of the LPXTG motif (Navarre et al., 1994). Amide linkage of the carboxyl of threonine to the cell wall crossbridge generates mature, anchored surface protein (M) (Schneewind et al., 1995). Surface protein processing was investigated by pulse-labeling polypeptides with [³⁵S]methionine. During the pulse, all three species, P1 and P2 precursors as well as mature Seb-Spa₄₉₀₋₅₂₄ can be detected. Within 1 min after the addition of the chase, most pulse-labeled surface protein was converted to the mature, anchored species. Surface protein anchoring was complete 3 min after the quenching of [³⁵S]methionine incorporation.

Sodium azide is an inhibitor of SecA, an essential component of the secretory pathway in bacteria (Oliver et al., 1990). Addition of 5 mM sodium azide to staphylococcal cultures 5 min prior to pulse-labeling significantly reduced protein export and led to the accumulation of leader peptide bearing P1 precursor (Schneewind et al., 1992). Methanethiosulfonates react with sulfhydryl (Akabas et al., 1995) and one of these compounds. [2-(trimethylammonium) ethyl]methanethiosulfonate) (MTSET) prevented incorporation of [³⁵S]methionine by staphylococci. However, when added 15 sec after the beginning of the pulse, MTSET interfered with the cleavage of sorting signals at the LPXTG motif, while the Sec-dependent export of P1 precursor remained unaltered. This result revealed that sortase must harbor a sulfhydryl that is necessary for enzymatic cleavage at LPXTG bearing sorting signals.

Sortase's requirement of sulfhydryl for enzymatic activity was tested by the addition of other sulfhydryl reagents and analysis of inhibition of the cleavage of sorting signals at the LPXTG motif. MTSES, another methanethiosulfonate, also interfered with sorting albeit not as effectively as MTSET. pHMB, an organic mercurial known to inhibit cysteine proteases, also displayed an inhibitory effect, whereas alkylating reagents such as N-ethylmaleimide, iodoacetate and iodoacetamide did not (Creighton, 1993). Sulfhydryl reducing agents, i.e. dithiothreitol and mercaptoethanol, did not affect the sorting reaction. Neither PMSF, which reacts with hydroxyl (Creighton, 1993), nor treatment with the divalent cation chelator EDTA interfered with cell wall sorting, indicating that sortase likely does not require divalent cations or hydroxyl for cleavage and anchoring of surface protein.

Antibiotic Inhibition of Bacterial Cell Wall Synthesis and Cell Wall Sorting.

To examine the effect of known antibiotics on cell wall sorting three compounds, penicillin, vancomycin and moenomycin were used. S. aureus OS2 (pSebSpa₄₉₀₋₅₂₄) was grown in minimal medium until A₆₀₀ of 0.3, treated with 10 μg/ml of either penicillin, vancomycin, or moenomycin and incubated for an additional 5 h. At 30 min intervals during this experiment, aliquots were withdrawn for measurements of surface protein sorting and cell wall synthesis. The effect of antibiotics on the rate of bacterial cell wall synthesis was determined as the ratio of [³H]lysine/[³H]leucine label incorporated into acid precipitable, pronase resistant peptidoglycan. Lysine is a component of peptidoglycan, whereas leucine is not. Hence, the ratio of incorporation of these two amino acids is a measure for cell wall synthesis. Surface protein anchoring was measured by pulse-labeling and quantified as the ratio between the concentration of P2 precursor [P2] and mature, anchored Seb-Spa₄₉₀₋₅₂₄ [M].

Addition of vancomycin, penicillin or moenomycin reduced the growth rate of staphylococci as compared to a mock treated control. While the rate of cell wall sorting precursor cleavage remained constant during the growth of mock treated staphylococci, the addition of vancomycin led to a steady accumulation of P2 precursor, indicating that this compound caused a reduction of the sorting reaction. A similar, albeit weaker effect was observed when moenomycin was added to staphylococcal cultures. In contrast, penicillin G did not alter the rate of cell wall sorting. As expected, all three antibiotics diminished the rate of peptidoglycan synthesis. Together these data revealed that vancomycin and moenomycin cause a reduction in the rate of cell wall sorting, while penicillin had no effect on surface protein anchoring.

Cell Wall Sorting in Staphylococcal Protoolasts

Previous work revealed that protoplasts, generated by muralytic digestion of staphylococci or penicillin selection of streptococcal L forms, secretec surface protein into the surrounding medium (van de Rijn et al., 1976). This can be explained in two ways. Either the C-terminal sorting signals cannot retain surface proteins in the envelope of protoplasts or the presence of intact, assembled cell wall is not required to cleave sorting signals at their LPXTG motif. To distinguish between these possibilities, the surface protein anchoring in intact bacteria and staphylococcal protoplasts was measured. Wild-type staphylococci cleaved the Seb-Cws-BlaZ precursor to generate the mature, anchored NH₂-terminal Seb and COON-terminal, cytoplasmic BlaZ fragments (Navarre et al., 1994). When tested in staphylococcal protoplasts generated by lysostaphin-digestion of the cell wall, precursor cleavage occurred similar to whole cells, indicating that the presence of mature, assembled cell wall is not required for cleavage of sorting signals. Unique sorting products in protoplasts that migrated more slowly than mature, anchored Seb were observed. As these species were immunoprecipitated with a-Seb but not with a-BlaZ, they likely represent products of the sorting reaction. The COON-terminal anchor structure of these protoplast species are distinct from those generated by lysostaphin-digestion (three glycyl attached to the carboxyl of threonine), as they migrated more slowly on SDS-PAGE than lysostaphin-released Seb.

To examine whether all cleaved Seb fragments were released into the extracellular medium, pulse-labeled protoplasts were sedimented by centrifugation and separated from the extra-cellular medium in the supernatant. All Seb-Cws-BlaZ precursor and COOH-terminal BlaZ cleavage fragment sedimented with the protoplasts. In contrast, NH₂-terminal Seb fragments that migrated at the same sped as Seb released by lysostaphin-digestion from the cell wall of intact staphylococci were soluble in the culture medium. Some, but not all, of the more slowly migrating Seb species sedimented into the pellet, suggesting that these products of the sorting reaction may be attacned to protoplast membranes. No precursor cleavage was observed for Seb-Cws_{DLPXTG}-BlaZ in either whole cells or staphylococcal protoplasts.

Materials and Methods

Bacterial Strains and Plasmids

Plasmids pSeb-Spa₄₉₀₋₅₂₄(3), pSeb-Csw-BlaZ, and pSeb-Cws_{DLPXTG}-BlaZ (Navarre, W. W. and Schneewind. O. Mol. Microbiol., 14:115-121, 1994) were transformed into S. aureus OS2 (spa:ermC, r) (Schneewind et al., 1992) and have been described previously. Staphylococci were generally grown in tryptic soy broth or agar. All chemicals were purchased from Sigma unless indicated otherwise.

Characterization of Cell Wall Sorting Intermediates

S. aureus OS2 (pSeb-Spa₄₉₀₋₅₂₄) was grown overnight in CDM (van de Rijn et al., 1980) (Jeol BioSciences) supplemented with chloramphenicol (10 mg/ml), diluted 1:10 into minimal medium and grown with shaking at 37° C. until A₆₀₀ 0.6. Cells were labeled with 100 mCi of [³⁵S]-Promix (Amersham) for 1 min. Labeling was quenched by the addition of an excess non-radioactive amino acid [50 ml chase (100 mg/ml casamino acids, 20 mg/ml methionine and cysteine)]. At timed intervals after the addition of the chase, 0, 1, 3, and 10 min, 250 ml aliquots were removed and protein, was precipitated by the addition or 250 ml 10% TCA. The precipitate was sedimented by centrifugation 15,000×g for 10 min. washed with 1 ml acetone and dried. Samples were suspended in 1 ml of 0.5 M Tris-HCl, pH 6.8 and staphylococcal peptidoglycan was digested by adding 50 ml lysostaphin (Schindler, C. A. and Schuhardt, V. T., Proc. Natl. Acad. Sci. USA, 51, 414-421, 1964) (100 mg, AMBI Pharmaceuticals) and incubating for 1 h at 37° C. Proteins were again precipitated with TCA, washed with acetone and subjected to immunoprecipitation with a-Seb followed by SDS-PAGE and Phosphorlmager analysis.

To characterize the P1 and P2 precursors, 1 ml of culture was either incubated with 5 mM sodium azide for 5 min prior to labeling or 5 mM MTSET was added 15 sec after the beginning of the pulse.

Antibiotic Inhibition of Cell Wall Sorting

Overnight cultures of S. aureus OS2 (pSeb-Spa₄₉₀₋₅₂₄) grown in CDM were diluted into fresh minimal medium and incubated for until A₆₀₀ 0.3. Cultures were then treated with either penicillin (10 mg/ml), vancomycin (10 mg/ml), moenomycin (10 mg/ml) or left untreated. A 0.5 ml culture sample was removed for pulse labeling with 100 mCi of [³⁵SJ-Promix (Amersham) for 5 min. Labeling was quenched and proteins precipitated by the addition of 0.5 ml 10% TCA. The precipitate was collected by centrifugation, washed in acetone and dried under vacuum. The pellets were suspended in 1. ml 0.5 M Tris-HCl, pH 7.0, 50 ml lysostaphin (100 mg/ml, AMBI Pharmaceuticals) added and the staphylococcal cell wall digested by incubating for 1 h at 37° C. Proteins were precipitated with TCA, washed in acetone, dried and solubilized in 50 ml 0.5 M Tris-HCl, pH 7.5, 4% SDS and boiled for 10 min. Aliquots of solubilized surface protein were immunoprecipitated with a-Seb followed by SDS-PAGE and Phosphorlmager analysis.

Peptidoglycan Synthesis Measurements

Staphylococci were grown in the presence or absence of antibiotics as described above. At 30 min intervals, 0.5 ml culture samples were withdrawn and labeled with either 50 mCi [³H]Iysine or 50 mCi [³H]leucine for 20 min (Boothby et al., 1971). All labeling was quenched by the addition of 0.5 ml 20% TCA. Samples were heated to 96° C. for 30 min. cooled to room temperature and pipetted onto glass fiber filters. The filters were placed into a holder and washed under vacuum suction with 25 ml 75% ethanol and 2 ml 50 mM Tris-HCl, pH 7.8. After incubation in 5 ml pronase solution (50 mM Tris-HCl, pH 7.8, 1 mg/ml pronase) at 30° C. for 30 min, filters were washed again with ml of distilled water and 4 ml ethanol. The amount of radioactivity retained by the filter was determined by scintillation counting (Boothby et al., 1971).

Chemical Inhibitors of the Sorting Reaction

S. aureus OS2 (pSeb-Spa₄₉₀₋₅₂₄) was grown overnight in CDM supplemented with chloramphenicol (10 mg/ml), diluted 1:10 into minimal medium and grown with shaking at 37° C. until A₆₀₀ 0.6. Cells were labeled with 100 mCi of [³⁵S]-Promix (Amersham) for 5 min. Chemicals were added to a final concentration of 5 mM 15 sec after the beginning of the pulse. All labeling was quenched by adding TCA to 10%. Precipitated cells and proteins were collected by centrifugation, washed in acetone aihd and the staphylococcal cell wall digested with lysostaphin as described above. The digests were again precipitated with TCA, immunoprecipitated with a-Seb followed by SDS-PAGE and Phosphorlmager analysis.

Cell Wall Sorting in Staphylococcal Protoplasts

Overnight cultures of S. aureus OS2 (pSeb-Cws-BlaZ) or S. aureus OS2 (pSeb-CwsDLPXTG-BlaZ) grown in CDM were diluted 1:10 into minimal medium and grown with shaking at 37° C. until A₆₀₀ 0.6. One ml of culture was pulse-labeled with 100 mCi of [³⁵S]-Promix (Amersham) for 2 min and labeling was quenched by the addition of 50 ml chase solution. Culture aliquots (0.5 ml) were removed for TCA precipitation either during the pulse or 20 min after the addition of chase. Another culture aliquot was first converted to protoplasts and then subjected to labeling. The cells were sedimented by centrifugation at 15,000×g for 5 min and suspended in 1 ml 50 mM Tris-HCl, 0.4 M sucrose, 10 mM MgC12, pH 7.5. The cell wall was digested with lysostaphin (100 mg) for 30 min at 37° C. The protoplasts were labeled with 100 mCi of [³⁵S]-Promix (Amersham) for 2 min and labeling quenched by the addition of 50 ml chase solution. For sedimentation analysis, pulse-labeled staphylococci were centrifuged at 15,000×g for 10 min to separate soluble surface protein from those that were bound to protoplasts. All samples were precipitated with TCA, washed in acetone and suspended in 50 ml 4% SIDS, 0.5 M Tris-HCl pH 7.5 with boiling for 10 min. Aliquots of solubilized surface protein precursor and anchored products were immunoprecipitated with a-Seb and a-BlaZ, subjected to SIDS-PAGE: and Phosphorlmager analysis.

### Example 3

### Purification and Characterization of Sortase-Transpeotidase

To examine whether staphylococcal sortase captures surface proteins after their cleavage at the LPXTG motif as acyl-enzyme intermediates, the proposed acylenzyme intermediates between surface protein and sortase were treated by hydroxylaminolysis (Lawrence et al., 1970; Kozarich et al., 1977). In this model, the sulfhydryl of sortase may function as a nucleophile at the peptide bonal between threonine and glycine, thereby forming a thioester with the carboxyl of threonine and releasing the amino of glycine. Lipmann first used hydroxylamine to demonstrate the existence of acyl-enzyme intermediates as this strong nucleophile attacks thioester to form hydroxamate with carboxyl, thereby regenerating enzyme sulfhydryl (Lipmann et al., 1945).

Hydroxylaminolysis of Surface Proteins

Hydroxylaminolysis of surface proteins was examined by pulse-labeling staphylococci with [³⁵S]methionine in either the presence or absence of 0.2 M NH₂OH. Cultures were labeled with [³⁵S]methionine and divided into two aliquots, each of which was precipitated with 5% TCA. One sample was boiled in hot SIDS, whereas the other was first treated with lysostaphin to release all anchored surface protein, and then boiled in hot SDS. Surface protein (SEB-SPA₄₉₀₋₅₂₄) of mock treated staphylococci was insoluble in hot SDS (3.8%) unless the peptidoglycan had been digested with lysostaphin prior to boiling in SDS (100%). Addition of 0.2 M NH₂OH caused 25.3% of all labeled SEB-SPA490-524 to be released into the extra-cellular medium and to be soluble in hot SDS. This phenomenon was not strain specific as S. aureus OS2 and S. aureus BB270 displayed similar amounts of surface protein hydroxylaminolysis.

If the solubility of surface proteins in hot SDS is caused by hydroxylaminolysis of acyl-enzyme intermediates, addition of NH₂OH after the pulse labeling of staphylococci should not release SEB-SPA₄₉₀₋₅₂₄ as this polypeptide is rapidly anchored to the cell wall. Addition of NH₂OH either before or during the pulse with [³⁵S]methionine released surface proteins into the extra-cellular medium (16.9% and 12.7% respectively) (FIG. 12B). Very little SDS-soluble SEB-SPA₄₉₀₋₅₂₄ was detected when NH₂OH was added after the pulse (4%). Increasing the amount of NH₂OH prior to pulse-labeling resulted in increased amounts of released surface proteins.

Characterization of NH₂OH-Released Surface Proteins

Hydroxylaminolysis of sortase acyl-intermediates should result in the formation of surface protein hydroxamate at the threonine of the LPXTG motif. To characterize NH₂OH-released surface protein, staphylococci (10¹³ cfu) expressing the surface protein SEB-MH₆-CWS (Ton-That et al., 1997) were incubated in the presence or absence of 0.1 M NH₂OH. Samples were centrifuged to sediment bacteria and SEB-MH₆-CWS was purified from the supernatant by affinity chromatography and analyzed on Coomassie-stained SDS-PAGE. Treatment with 0.1 M NH₂OH caused the release of SEB-MH₆-CWS by S. aureus strains OS2 and-BB270. SEB-MH₆-CWS purified from strain BB270 was cleaved at methionine with cyanogen bromide. COOH-terminal peptides bearing anchor structures were purified by affinity chromatography and analyzed by rpHPLC (Ton-That et al., 1997). The chromatogram of anchor peptides released from mock treated bacteria revealed a major absorbance peak at 29% CH₃CN (FIG. 13B). The sample was subjected to electrospray-ionization mass spectrometry (ESI-MS) and a compound with an average mass of 2236 Da was detected. This measurement is consistent with the structure of anchor peptide linked to a branched cell wall tetrapeptide [L-Ala-D-iGln-L-Lys(NH₂H₆AQALPET-Gly₅)-D-Ala, predicted mass 2235]. This surface protein species is not linked to the glycan strands of the staphylococcal cell wall and is therefore released into the culture medium. The chromatogram of anchor peptides released by treatment with 0.1 M NH₂OH revealed a major absorbance peak at 32% CH₃CN. ESI-MS identified a compound with the average mass of 1548 Da. When subjected to Edman degradation, the peptide sequence NH₂-H₆AQALPET* was obtained, in which the thirteenth cleavage cycle released a phenylthiohydantoin moiety of unknown structure. The predicted mass of NH₂-H₆AQALPET> (T> indicates threonine hydroxamate) is 1565 Da, 17 Da more than the observed mass of 1548 Da. Fractions of both chromatograms were scanned by rpHPLC for the presence of ion signals with an average mass of 1548, 1565 or 2236. rpHPLC fractions of anchor peptides from mock-treated cultures contained the compound with mass 2236, however no ions of the predicted mass 1548 or 1565 were detected. In contrast, rpHPLC fractions collected from anchor:peptides of NH₂OH-treated staphylococci harbored compounds with an average mass of 1548 Da (NH₂-H₆AQALPET*, 32% CH₃CN) and 1565 Da (NH₂H₆AQALPET>, 31% CH₃CN), but not the anchor peptide of 2235 Da. Thus, treatment with 0.1 M NH₂OH released surface protein from staphylococci as a hydroxamate of the threonine within) the LPXTG motif, suggesting that sortase forms an acyl-enzyme intermediate with cleaved surface protein. The peptide NH₂-H₆AQALPET> appears to be unstable during our purification, thereby generating NH₂-H₆AQALPET* with a loss of 17 Da at the threonine hydroxmate.

Analysis of Sortase Hydroxylaminolvsis Activity In Vitro in the Presence of NH₂OH

If NH₂OH can release surface protein from staphylococci in vivo, sortase may catalyze the cleavage of LPXTG motif bearing peptides in the presence of NH₂OH in vitro. Fluoresence of the EDANS fluorophore within the peptide DABCYL-QALPETGEE-EDANS is quenched by the close proximity of DABCYL (Wang et al., 1990). When the peptide is cleaved and the fluorophore separated from DABCYL, an increase in fluorescence is observed (Matayoshi et al., 1989). Incubation of the LPXTG peptide with crude staphylococcal extracts caused only a small increase in fluorescence. However, the addition of 0.1 M NH₂OH to staphylococcal extracts resulted in a forty fold increase in fluorescence intensity. This activity appears to be specific for sortase as it can be inhibited by pre-incubation of staphylococcal extracts with methanethiosulfonate (MTSET) (Smith et al., 1975), a known inhibitor of the sorting reaction. These results suggest that sortase catalyzes the hydroxylaminolysis of LPXTG peptide in vitro. Thus, surface protein is cleaved between the threonine and the glycine of the LPXTG motif, resulting in the formation of a NH₂OH-sensitive thioester linkage between the carboxyl of threonine and the active site sulfhydryl of sortase. In vivo, the acyl-enzyme intermediate is resolved by a nucleophilic attack of the amino within the pentaglycine crossbridge. Recent observations suggest that the pentaglycine crossbridge of the lipid II precursor functions as a nucieophile for the sorting reaction. We show here that hydroxylamine can substitute for pentaglycine both in vivo and in vitro.

Purification and Characterization of Sortase

When expressed in E. coli and analyzed by centrifugation of crude lysates, the staphylococcal SrtA protein sedimented with membranes. To obtain a soluble enzyme and to examine its properties, the NH₂-terminal membrane anchor segment of SrtA was replaced with a six histidine tag (SrtA_{DN}). SrtA_{DN} was expressed in E. coli XL-1 Blue and purified by affinity chromatography from cleared lysates. When incubated with the LPXTG peptide and measured as an increase in fluorescence, SrtA_{DN} catalyzed cleavage of the substrate. Addition of 0.2 M NH₂OH to this reaction resulted in an increase in fluorescence, indicating that cleavage of the LPXTG peptide occurred more efficiently. Hydroxylaminolysis of LPXTG peptide was dependent on the sulfhydryl of SrtA_{DN} as pre-incubation with MTSET abolished all enzymatic activity. Methanethiosulfonate forms disulfide with sulfhydryl (Smith et al., 1975; Akabas et al., 1995) which can be reversed by reducing reagents such as dithiothreitol (DTT) (Pathak et al., 1995). MTSET-inactivated SrtADN was incubated in the presence of 10 mM DTT, which restored 80% of LPXTG peptide cleavage activity. The availability of purified, soluble sortase (SrtA_{DN}) and an in vitro assay for the hydroxylaminolysis of LPXTG peptide should allow the screening for compounds that interfere with the anchoring of surface protein in Gram-positive bacteria. Such compounds may be useful for the therapy of human infections with Gram-positive bacteria that have gained resistance to all known antibiotics.

Materials and Methods

Pulse-Chase Screen of Hydroxylaminolvsis of Surface Proteins

Staphylococci were grown in minimal medium until OD₆₀₀ 0.6 and pulse-labeled with 100 μCi Pro-Mix ([³⁵S] methionine and cysteine) for 1 min. Incorporation of radio-label into polypeptides was quenched by the addition of 50 μl chase solution (100 mg/ml casamino acids, 20 mg/ml methionine and cysteine) and incubation was continued at 37° C. for 5 min. Two 0.5 ml aliquots of labeled culture were each precipitated with 0.5 ml 10% TCA, washed in acetone and dried under vacuum. One sample was suspended in 50 μl 0.5 M tris, 4% SDS and boiled. The other sample was first suspended in 1 ml 0.5 M Tris Ph 7.0 and the cell wall digested for 1 h at 37° C. by adding 50 μL 2 mg/ml lysostaphin. The sample was precipitated with 75 μl 100% TCA, washed in acetone, dried and then boiled in SDS. Aliquots were subjected to immunoprecipitation with a-SEB and analyzed after SDS-PAGE on Phosphorlmager.

Purification of NH₂OH Surface Proteins

Staphylococci (10¹³ cells) were incubated in 200 ml 50 mM Tris-HCl, pH 7.0 with or without 0.1 M NH₂OH for 60 min. Samples were centrifuged at 10,000×g for 15 min and the supernatants applied to 1 ml Ni-NTA column pre-equilibrated with column buffer (CB, 50 mM Tris-HCl, 15 mM NaCl, pH 7.5). The column was washed first with 20 ml CB and 2 ml C8 containing 10% glycerol and eluted with 4 ml of column buffer and 0.5 imidazol. Aliquots were mixed with sample buffer and separated on SDS-PAGE. The eluate was precipitated with TFA (10%), washed in acetone and dried under vacuum. The sample was suspended in 600 μl 70% formic acid and, after addition of a crystal of cyanogen bromide, incubated overnight. Cleaved peptides were repeatedly dried and suspended in water to evaporate cyanogen bromide, solubilized in 1 ml buffer A and subjected to affinity chromatography as previously described. Peptides were eluted in 4 ml of 6 M guanidine-hydrochloride, 0.2 M acetic acid, desalted over C18 cartridge and dried. Pellets were solubilized in 50 μl buffer B (8 M urea, 50 mM phosphate, 10 mM Tris-HCl, pH 7.3) and subjected to rpHPLC on C18 column (Hypersil, Keystone Scientific) with a linear gradient from 1%-99% CH₃CN in 0.1% TFA in 90 min. MALDI-MS and ESI-MS was performed as described (Ton-That et al., 1997).

Identification of Peptide Structure by Mass Spectrometry

The structure or the peptides with mass 1548 and 1565 was determined by tandem mass spectrometry, MS/MS using the parent ions. Collisionally induced dissociation of the parent ions produced daughter ion spectra consistent with compound structures NH₂-H₆AQALPET> (T> is threonine hydroxamate, predicted compound mass 1565) and NH₂-H₆AQALPET* (T> represents a loss of 17 Da of threonine hydroxamate; the structure of this residue is unknown).

Assay of Sortase Activity by Fluorescent Assay

Reactions were assembled in a volume of 120 μI containing 50 mM Tris-HCl, 50 mM NaCl, pH 7.5. The concentration of LPXTG peptide substrate (DABCYL-QALPETGEE-EDANS) was 10 μM, of MTSET 5 mM, of NH₂OH 0.2 M. Staphylococcal cell extracts were obtained by subjecting 10¹³ cells to disruption in a bead beater instrument. The crude extract was subjected to slow speed centrifugation at 3,000×g for 15 min to remove beads and intact cells. A 10 μl aliquot of the supernatant, containing approximately 50 mg/ml protein, was used as enzyme preparation. Incubations were carried out for 1 h at 37° C., followed by centrifugation of the sample at 15,000×g for 5 min. The supernatant was subjected to analysis in a fluorimeter using 395 nm for excitation and 495 nm for recordings.

Purification of Sortase by Addition of Histidine Tag

The primers orf6N-ds-B (5′-AAAGGATCCAAACCACATATCGATAATTATC-3′) and orf6C-dT-B (5′-AAAGGATCCTTTGACTTCTGTAGCTACAAAG-3′) were used to PCR amplify the srtA sequence from the chromosome of S. aureus OS2. The DNA fragment was cut with BamHI, inserted into pQE16 (Qiagen) cut BamHI to generate pHTT5, transformed into E. coli XL-1 Blue and selected on Luria broth with ampicillin (100 μg/ml). E. coli XL-1 Blue (pHTT5) (10¹² cells) were suspended in 30 ml C buffer (50 mM Bis-Tris-HCl, 150 mM NaCl, 10% glycerol, pH 7.2) and lysed by one passage through a French pressure cell at 14,000 psi. The extract was centrifuged at 29,000×g fcr 30 min and the supernatant applied to 1 ml Ni-NTA resin, pre-equilibrated with C buffer. The column was washed with 40 ml C buffer and SrtA_{DN} protein was eluted in 4 ml C buffer with 0.5 M imidazol at a concentration of 30 μg/μl.

Reactions were assembled in a volume of 260 μl containing 50 mM Hepes buffer, 150 mM NaCl, pH 7.5 and as indicated 5 μM SrtA_{DN} in 50 mM BisTris, pH 7.5, 10 μM LPXTG peptide (DABCYL-QALPETGEE-EDANS), 10 uM TGXLP peptide (DABCYL-QiATGELPEE-EDANS), 5 M MTSET, 0.2 M NH₂OH, 5 mM pHMB or 10 mM DTT. Incubations were carried out for 1 h at 37° C. Samples were analyzed in a fluorimeter using 395 nm for excitation and 495 nm for recordings.

### Example 4

### Identification of a Second Sortase Gene srtB

A second sortase gene, srtB, was identified with Blast searches using the srtA gene as query (SEQ ID NO:1). All S. aureus strains examined had both srtA and srtB genes. The srtB gene (SEQ ID NO:3) specifies a polypeptide chain of 244 amino acids (SEQ ID NO:4). Alignment of SrtB and SrtA amino acid sequences indicates that SrtB has 22% identity and 37% similarity with the sequence of SrtA as well as 11 conserved amino acid residues. This degree of identity and similarity are the degree of identity and similarity determined with the Blast program (Tatusova et al., 1999).

Role of Multiple Sortase Enzymes in Staphylococci

The N-terminal membrane anchor segment of SrtB (residues 2-25) were replaced with a six-histidine tag (SrtBDN). In the absence of the peptidoglycan substrane, SrtA DN catalyzes peptide bond hydrolysis and cleaves LPETG peptide, presumably between the threonine and the glycine (Ton-That et al., 2000). This reaction was inhibited with methylmethane thiosulfonate, indicating that SrtB sortase catalyzes peptide bond hydrolysis and transpeptidation reaction, also via the conserved cystein residue.

S. aureus knockout variants were generated by replacing the srtB gene of wild-type S. aureus Newman with the ermC marker gene (strain SKM9). Elimination of the srtB did not result in a defect in cell wall anchoring of surface proteins such as: protein A. FnbA, FnbB or ClfA. However, it is likely that srtB mutant staphylococci display a sorting defect for some of the remaining surface proteins. Thus, SrtB and SrtA catalyze similar reactions using different surface protein substrates. It is possible that different sortase enzymes modify specific secretion pathways. For example, SrtA with the Sec-1 secretion pathway and SrtB with the Sec-2 secretion pathway, or vice-versa. Presence of multiple sets of secretion, signal peptidase and sortase genes in S. aureus indicate existence of more than one pathway for surface protein transport.

Effect of srtB Knockout Variant S. aureus on in Vivo Infectivity

The in vivo activity of srtB mutant staphylococci was determined using a kidney staphylococcal-abscess assay. S. aureus Newman and the srtB mutant, isogenic srtB:ermC knockout variant SKM7 were injected into the tail vein of Balb/c mice. Infection was allowed to proceed for 5 days. On day 5, all infected animals were euthanized, and their kidneys excised and homogenized. Kidney homogenates were then plated on tryptic soy agar plates. The level of staphylococcal infection in each animal, resulting from either the wild type (wt) or mutant strain was then correlated with the number of staphylococci obtained per kidney.

### Example 5

### Identification and Function of Sortase Genes and Their Substrates

Genome sequencing and homology searches have shown that gram-positive pathogens encode at least two, in some cases up to seven sortase genes (Mazmanian et al., 2001; Pallen et al., 2001). This Example concerns investigating the roles of multiple sortase genes. The present inventors contemplated that if each sortase anchored distinct surface protein species to the cell wall envelope, sortase genes encoding diverse substrates in the microbial genome sequence may be identified (Ton-That et al., 2001).

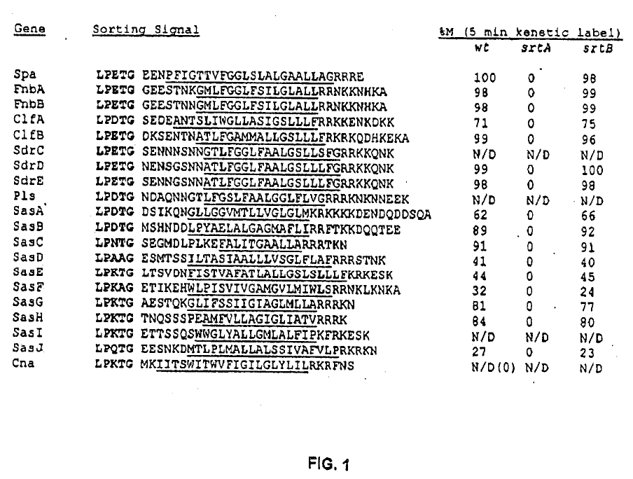

Accordingly, Blast searches were performed using the genomes of the S. aureus strains Mu50, N315, COL and NTCC8325-4 (Kuroda et al., 2001). Initial searches were performed using the protein A sorting signal to identify sorting signals. Subsequent searches utilized the initially identified sorting signals to perform further queries. Twenty genes, comprising sorting signal signals which also comprise a LPXTG motif were identified. In the LPXTG motif, X may be a glutamic acid (E), an aspartic acid (D), a lysine (K), an alanine (A) or an asparagine (N) residue (FIG. 1) (Mazmanian et al., 2001).

To test whether the newly identified sorting signals were substrates for SrtA or SrtB, isogenic mutants of S. aureus RN4220 (wild-type) were generated that lacked either srtA or srtB (Mazmanian et al., 2000). Staphylococcal strains were transformed with plasmids that encoded hybrid proteins in which each sorting signal had been fused to the C-terminal of enterotoxin B (Seb), a protein that is normally secreted into the extracellular environment (Scheenwind et al., 1993). Anchoring of hybrid proteins to the cell wall envelope was measured by determining the mobility of pulse-labeled precursor proteins and their cleavage products on SDS-PAGE. Transport of the P1 precursor by the Sec pathway results in cleavage of the N-terminal signal peptide and generates the P2 species. Sortase cleaves the P2 precursor and anchors the mature (M) polypeptide to the cell wall envelope (Ton-That and Schneewind, 1999). When analyzed in wild-type S. aureus, sixteen Seb hybrids were cleaved to generate P2 and mature (M) anchored species (FIG. 1). A staphylococcal mutant with a chromosomal deletion of srtA failed to cleave the P2 precursor species of all sixteen fusions, indicating that sorting signals with a LPXTG motif require SrtA for their anchoring to the cell wall envelope (FIG. 1). In contrast, mutants with a deletion of srtB anchored LPXTG motif containing precursor proteins to the cell wall in a manner that was indistinguishable from that of wild-type staphylococci (FIG. 1).

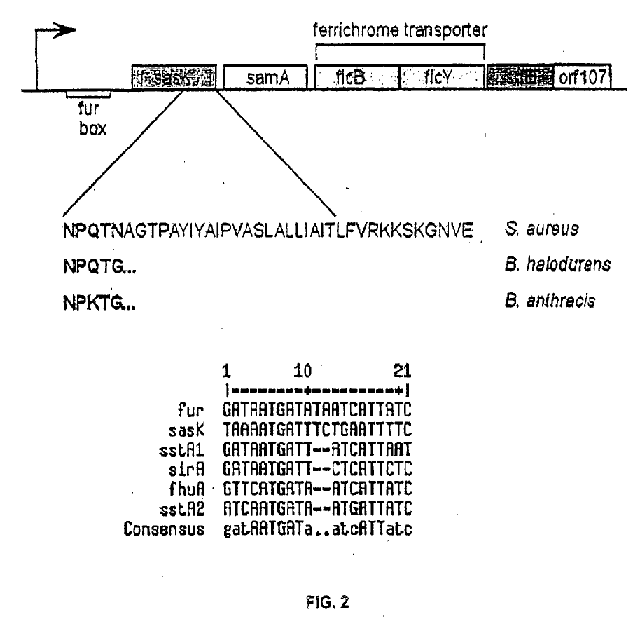

To identify sorting substrates of SrtB, the LPXTG motif was removed from sorting signal queries and homologous gene sequences were identified with Blast searches. One of the genes so identified is the sasK gene which encodes a 227 residue polypeptide with a N-terminal signal peptide and a C-terminal hydrophobic domain and charged tail, resembling the sorting signal of SasG. sasK (Staphylococcus aureus surface protein K) maps on the chromosome to the same operon as srtB as evidenced by the presence of short intervening sequences or short overlaps between open reading frames (FIG. 2). Initial analysis shows that the sasK/srtB operon comprises five open reading frames, sasK-samA-ficB-ficY-srtB-orf107, with samA encoding a presumed type I membrane protein and ficB and ficY specifying a putative ferrichrome ABC binding protein (a lipoprotein) and ferrichrome transport permease, respectively (Braun, 1998; Koster, 1991). The orf107 gene encodes a 107 amino acid polypeptide of unknown function that is presumably located in the bacterial cytoplasm (FIG. 2). The promoter region of the sasK/srtB operon encompasses a canonical nucleotide-binding site for Fur, the ferric uptake regulator (Xiong et al., 2000) (FIG. 2). Fur is a DNA binding protein that prevents transcription of Fur-regulated genes when environmental iron concentrations are high. To test whether expression of sasK/srtB is controlled by Fur, staphylococci were grown in media with or without iron and expression measured by immunoblotting with SrtB-specific antibody. SrtB was expressed in the absence but not in the presence of iron. Further, deletion of the fur gene caused mutant staphylococci to express srtB both in the presence and absence of iron. Together, these results suggest that expression of the sasK/srtB operon is controlled by Fur and is induced under iron limiting conditions, as is typically encountered when invasive pathogens such as S. aureus enter a human host. srtA was expressed both in the presence or absence of iron and its concentration was not affected by deletion of fur or srtB.

To test whether SasK is a substrate for SrtB-mediated anchoring to the cell wall envelope, a mutant gene with a DNA insertion encoding a FLAG epitope tag upstream of the C-terminal hydrophobic domain, SasK_{FLAG}, was constructed (FIG. 3). The cell wall of staphylococci expressing SasK_{FLAG}, was digested with two different enzymes and SasK_{FLAG} was detected by immunoblotting. Lysostaphin cuts the pentaglycine crossbridge of the cell wall (Schindler and Schuhardt, 1964), close to the anchoring point of protein A or of other surface proteins bearing the LPXTG motif. Mutanolysin, a N-acetylmuramidase (Yokogawa et al., 1974), cleaves the β1-4 glycosidic bond between N-acetylmuramic acid and N-acetylglucosamine. Mutanolysin releases protein A as a spectrum of fragments with linked peptidoglycan, all of which migrate more slowly on SDS-PAGE than the lysostaphin-released counterpart (Schneewind et al., 1993). When expressed in S. aureus RN4220 under iron-replete conditions, the mobility of SasK_{FLAG} on SDS-PAGE was not altered after digesting the cell wall with lysostaphin or muramidase. However, expression of SasK_{FLAG} in the fur mutant caused some, but not all, SasK_{FLAG} to migrate faster on SDS-PAGE than the P2 species observed in wild-type staphylococci. These species represent mature, cell wall anchored surface protein, as solubilization with mutanolysin resulted in their altered (slowed) mobility on SDS-PAGE. Cell wall anchoring of SasK_{FLAG} is dependent on srtB, as neither srtB nor srtB/fur double mutants generated the mature species. Further, over-expression of SrtB in fur mutants caused all SasK_{FLAG} to be linked to the cell wall, while over-expression of SrtB in wild-type cells resulted in cell wall anchoring of some, but not all SasK_{FLAG}. Thus, SasK is a cell wall anchored surface protein that requires srtB sortase for proper attachment to the envelope.

### Example 6

### Anchoring Function of the Sorting Signals

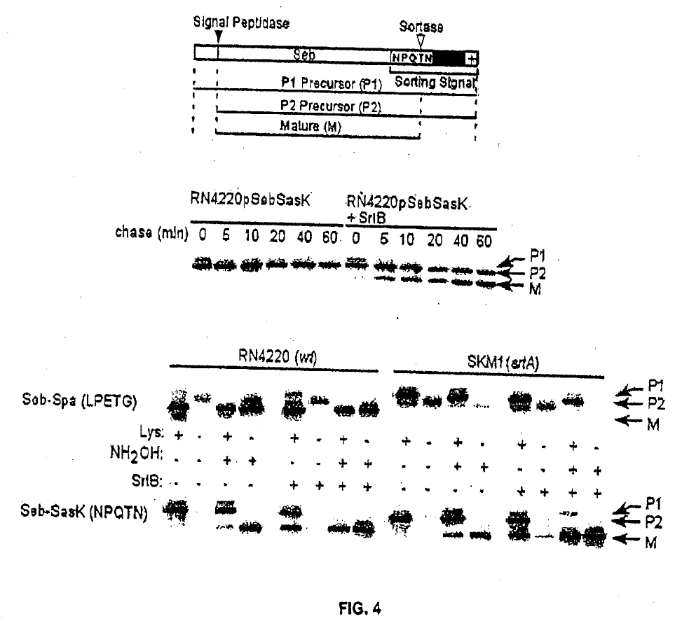

The present inventors also investigated if the sorting signal of SasK is capable of anchoring a secreted protein to the cell wall envelope. Anchoring of the Seb-SasK sorting signal fusion occurred only in staphylococci that over-expressed SrtB but not in wild-type cells or in srtB mutants that were grown in the presence of iron (FIG. 4). The cell wall anchoring of a Seb-Spa sorting signal fusion was not affected by deletion or over-expression of srtB, however deletion of srtA abrogated cell wall anchoring of this hybrid polypeptide (FIG. 4). Thus, when tethered to a secreted polypeptide, the sorting signal of SasK is sufficient to anchor the hybrid protein to the cell wall, provided that srtB sortase is expressed at sufficiently high levels.

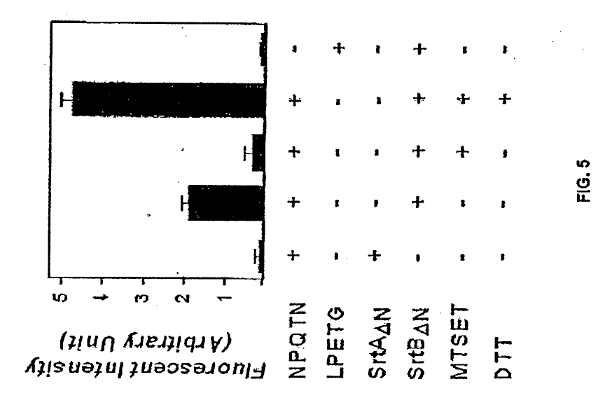

SrtA_{ΔN} and SrtB_{ΔN}, recombinant sortases with a six histidine tag replacing the N-terminal membrane anchor, were purified from E. coli extracts using affinity chromatography and were incubated with peptide substrates containing either the LPXTG or the NPQTN sequence motifs (FIG. 5). As expected, purified SrtA_{ΔN}, but not purified SrtB_{ΔN}, cleaved the LPXTG containing peptides (Ton-That et al., 1998; Ton-That 1999). In contrast, purified SrtB_{ΔN}, but not SrtA_{ΔN}, cleaved the NPQTN peptides. Both enzymes, SrtA_{ΔN} and SrtB_{ΔN}, were inhibited by incubation with methylmethane thiosulfonates or p-mercury benzoate, indicating that SrtB, similar to SrtA, utilizes an active site cysteine as a nucleophile to cleave its substrate.

To analyze the role of srtB and SrtB-mediated anchoring of surface protein during infection, the virulence of staphylococcal strains was assessed using a murine renal abscess model (Albus et al., 1991). Mice were injected into the tail vein with 10⁷ cfu of S. aureus Newman, a clinical human isolate, or isogenic derivatives lacking srtA (SKM12), srtB (SKM7) or srtA/srtB (SKM14). Mice were sacrificed five days after infection and homogenized kidneys were analyzed for abscess formation by plating on agar medium and colony formation (FIG. 6). As reported previously, srtA mutant S. aureus displayed a two-log reduction in virulence (>99% reduction of bacteria in kidneys) as compared to wild-type strain Newman (Mazmanian et al., 2000). No discernable defect in pathogenicity was detected for the srtB mutant strain, while srtA/srtB mutant staphylococci displayed a reduction in virulence similar to srtA mutants. Incubation of mice for a prolonged period of nine days, revealed a significant reduction in the number of srtB mutant staphylococci within infected kidneys as compared to strain Newman. Thus, although srtB may not be involved in the initial establishment of S. aureus disease, the present data indicates that srtB is required for staphylococcal persistence in infected tissues.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references and the references identified in the text, to the extent that they provide exemplary procedural or other details supplementary to those set forth herein, are specifically incorporated herein by reference.

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,162,282
U.S. Pat. No. 4,275,149
U.S. Pat. No. 4,277,437
U.S. Pat. No. 4,310,505
U.S. Pat. No. 4,533,254
U.S. Pat. No. 4,683,195
U.S. Pat. No. 4,683,202
U.S. Pat. No. 4,728,575
U.S. Pat. No. 4,728,578
U.S. Pat. No. 4,737,323
U.S. Pat. No. 4,797,368
U.S. Pat. No. 4,800,159
U.S. Pat. No. 4,883,750
U.S. Pat. No. 4,921,706
U.S. Pat. No. 5,028,592
U.S. Pat. No. 5,139,941
U.S. Pat. No. 5,279,721
U.S. Pat. No. 5,432,260
U.S. Pat. No. 5,786,214
U.S. Pat. No. 5,840,317
U.S. Pat. No. 5,840,873
U.S. Pat. No. 5,843,640
U.S. Pat. No. 5,843,651
U.S. Pat. No. 5,843,663
U.S. Pat. No. 5,846,708
U.S. Pat. No. 5,846,717
U.S. Pat. No. 5,846,726
U.S. Pat. No. 5,846,729
U.S. Pat. No. 5,849,481
U.S. Pat. No. 5,849,486
U.S. Pat. No. 5,849,487
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,861,244
U.S. Pat. No. 5,863,732
U.S. Pat. No. 5,863,753
U.S. Pat. No. 5,866,331
U.S. Pat. No. 5,871,727
U.S. Pat. No. 5,879,703
U.S. Pat. No. 5,882,654
U.S. Pat. No. 5,900,481
U.S. Pat. No. 5,905,024
U.S. Pat. No. 5,910,407
U.S. Pat. No. 5,912,124
U.S. Pat. No. 5,912,145
U.S. Pat. No. 5,919,626
U.S. Pat. No. 5,919,630
U.S. Pat. No. 5,925,517
U.S. Pat. No. 5,928,862
U.S. Pat. No. 5,928,869
U.S. Pat. No. 5,929,227
U.S. Pat. No. 5,932,413
U.S. Pat. No. 5,935,791
U.S. Pat. No. 5,939,277
U.S. Pat. No. 6,107,090
U.S. Pat. No. 6,135,965
U.S. Pat. No. 6,214,874
U.S. Pat. No. 6,238,659
U.S. Pat. No. 6,245,568
U.S. Pat. No. 6,258,576
EPA No. 320,308
EPA No. 329,822
EPO 0273085
GB Application No. 2202328
GB Application No. 2193095 A
PCT Application No. PCT/US85/01161
PCT Application No. PCT/US87/00880
PCT Application No. PCT/US89/01025
PCT Application No. PCT/US89/05040;
PCT Application WO 88/10315
PCT Application WO 89/06700
WO 90/07641
WO 98/0748
WO 99/18933
Abbondanzo, et al., Am J Clin Pathol., 93(5):698-702, 1990.
Abe, et al., Neurosci Res., 38(4):325-9, 2000.
Aichele, et al., J Exp Med., 171(5):1815-20, 1990.
Akabas et al., Biochemistry, 34:12496-12500, 1995.
Aksentijevich et al., Hum Gene Ther., 7(9):1111-22, 1996.
Albus et al., Infect. Immun., 59:1008-1014, 1991.
Allred, et al., Arch Surg. 1990 January; 125(1):107-13. Review.
Altman et al., Science, 274:94-96, 1996.
Baichwal et al., “Vectors for gene transfer derived from animal DNA viruses: Transient and stable expression of transferred genes,” In: Gene Transfer, Kucherlapati, R., ed.,
Bajorin, et al., J Clin Oncol., 6(5):786-92, 1988.
Bangham, et al., J. Mol. Biol., 13:238-252, 1965.
Barany, et al., “Solid-Phase Peptide Synthesis,” In: The Peptides: Analysis, Synthesis, Biology, Gross and Meinhofer, eds., Academic Press, New York, pp. 3-284, 1980.
Bartlett et al., Biochemistry, 22:4618-4624, 1983.
Berger et al., “Guide to Molecular Cloning Techniques” (Methods in Enzymology, vol. 152, Academic Press, Inc., San Diego, 1987).
Bevan, et al., Nature, 342(6249):478-9, 1989.
Birner, et al., Mod. Pathol., 14(7):702-9, 2001.
Bjorck et al., Nature, 337:385-386, 1989.
Bodanszky, “Peptide Chemistry: A Practical Testbook” (2d ed., Springer-Verlag, Berlin, 1993).
Bolken et al., Infect. Immun., 69:75-80, 2001.
Boothby et al., Anal. Biochem. 44:645, 1971
Boussif et al., Proc. Nat'l Acad. Sci. USA, 92:7297-7301, 1995.
Braun, Science, 282:2202-2203, 1998.
Brinton, et al., In: N. Munoz, F. X. Bosch, K. V. Shah, and A. Meheus (eds.), The Epidemiology of Cervical Cancer and Human Papillomavirus (IARC Scientific Publications, 119), pp. 3-23. Lyon: Oxford University Press, 1992.
Brocklehurst et al., “Cysteine Proteases,” in New Comprehensive Biochemistry, vol. 16: Hydrolytic Enzymes (A. Neuberger and K. Brocklehurst, eds., Elsevier. New York, 1987), ch. 2, pp. 39158.
Brown, et al., Am J Vet Res., 51(9):1476-80, 1990.
Canfield et al., Methods in Enzymology, 189, 418-422, 1990.
Capaldi, et al., Biochem. Biophys. Res. Comm., 74(2):425-433, 1977.
Casement, et al., Virology, 211(1):261-7, 1995
Cason, et al., Int. J. Cancer, 50: 349-355, 1992.
Chang et al., Hepatology, 14:134A, 1991.
Chatterjee et al., Proc. Natl. Acad. Sci. USA, 51:9-16, 1964.
Chen, et al., Mol. Cell Biol., 7:2745-2752, 1987.
Cheng, et al., Investigative Radiology, vol. 22, pp. 47-55 (1987).
Clark et al., Human Gene Therapy, 6:1329-1341. 1995
Clave, et al., Diagn. Mol. Pathol., 9(3) 145-150, 2000.
Clerici, et al., J. Natl. Cancer Inst., 89: 245-250, 1997.
Coffin, In: Virology, Fields et al., eds., Raven Press, New York, pp. 1437-1500, 1990.
Cossart et al., Proc. Nat'l. Acad. Sci. USA, 97:5013-5015, 2000.
Coupar et al., Gene, 68:1-10, 1988.
Creighton, “Proteins”, W. H. Freeman and Company, New York, 1993
Crowe, et al., AIDS Res Hum Retroviruses, 3(2):135-45, 1987.
da Costa, et al., Biocell, 23(1): 65-72, 1999.
De Gruijil, et al., J. Gen. Virol. 77, 2183-2191, 1996.
De Jager, et al., Semin Nucl Med. 1993 April;23(2):165-79. Review.
de Jonge et al., J. Biol. Chem., 267:11248, 1992
Deamer, et al., in Liposomes (M. Ostro, ed.), Marcel Dekker, Inc., New York (1983), pp. 27-52.
Deres, et al., Nature. 1989 Nov. 30; 342(6249):561-4.
Dhar et al., Biochemistry, 39:3725-3733, 2000.
Doolittle, et al., Methods Mol. Biol. 1999; 109:215-37. Review
Dufour et al., Biochemistry, 34:9136-9143, 1995.
el Gorab, et al., Biochim Biophys Acta., 306(1):58-66, 1973.
Fechheimer et al., Proc. Nat'l Acad. Sci. USA, 84:8463-8467, 1987.
Felgner et al., Proc Natl Acad Sci USA., 84(21):7413-7, 1987.
Feltkamp, et al., Eur. J. Immunol., 23: 2242-2249, 1993.
Fendler et al., Catalysis in Micellar and Macromolecular Systems, Academic Press, New York, 1975.
Fischetti et al., Mol. Microbiol., 4:1603-1605, 1990.
Flock et al., EMBO J., 6:2351-2357, 1987.
Flotte et al., Gene Therapy, 2:29-37, 1995.
Flotte, et al., Am. J. Respir. Cell Mol. Biol., 7:349-356, 1992.
Fraley et al., Proc. Nat'l Acad. Sci. USA, 76:3348-3352, 1979.
Fraley, et al., Trends Biochem. Sci., 6:77, 1981
Friedmann, Science, 244:1275-1281, 1989.
Frohman, PCR Protocols: A Guide To Methods and Applications, Academic Press, New York, 1990.
Fujishima, et al., Cytometry., 24(4):382-9, 1996.
Gabizon et al., Cancer Res., 50(19):6371-8, 1990.
Ghosh, et al., In: Wu G. Wu C ed., Liver diseases, targeted diagnosis and therapy using specific receptors and ligands, New York: Marel Dekker, pp. 87-104, 1991.
Gilliland et al., Cancer Res., 40(10):3564-9, 1980.
Gloeckner, et al., J. Immunol. Methods, 252(1-2):131-8, 2001.
Goeddel, ed., “Gene Expression Technology” (Methods in Enzymology, vol. 185, Academic Press, Inc., San Diego 1991).
Gopal, Mol. Cell Biol., 5:1188-1190, 1985.
Graham et al., J. Gen. Virol., 36:59-72, 1977.
Graham, et al., Virology, 52:456-467, 1973.
Gregoriadis, et al., Biochem Biophys Res Commun., 89(4):1287-1293, 1979.
Gregoriadis, G., ed., Liposome Technology, vol. I, pp. 30-35, 51-65 and 79-107, CRC Press Inc., Boca Raton, Fla., 1984.
Grunhaus, et al., Seminar in Virology, 3:237-252, 1992.
Gulbis, et al., Hum Pathol. 1993 December;24(12):1271-85. Review.
Hamsikova, et al., J. Infect. Dis., 170: 1424-1431, 1994
Hara et al., Biochim Biophys Acta., 1278(1):51-8, 1996
Harland, et al., J. Cell Biol., 101:1094-1099, 1985.
Harlow et al., “Antibodies: A Laboratory Manual” (Cold Spring Harbor Laboratory, 1988).
Heath, et al., Chem. Phys. Lipids, 40:347, 1986.
Hermonat, et al., Proc. Nat'l Acad. Sci. USA, 81:6466-6470, 1984.
Hope et al., Biochimica et Biophysica Acta, 812: 55-65, 1985.
Horwich et al. J. Virol., 64:642-650, 1990.
Irie, et al., Lancet, 1(8641):786-7, 1989.
Irie, et al., Proc Natl Acad Sci USA, 83(22):8694-8, 1986.
Javitch et al., Neuron, 14:825-831, 1995.
Jha, et al., Lancet, 341: 1116-1118, 1993.
Jonsson et al., Eur. J. Biochem., 202:1041-1048, 1991.
Kadish, et al., J. Natl. Cancer. Inst. 89:1285-1293, 1997.
Kandler et al., Cell Mol. Life Sci., 54:305-308, 1998.
Kaplitt et al., Nature Genetics, 8:148-154, 1994
Kast, et al., Immunol Lett., 30(2):229-32, 1991.
Kast, et al., J. Immunotherapy, 14: 115-120, 1993.
Kopp et al., Microb. Drug Resist., 2:29, 1996
Koster, Biol. Met., 4:23-32, 1991.
Kotin et al., Proc. Nat'l Acad. Sci. USA, 87:2211-2215, 1990.
Koutsky, et al., N Engl J. Med. 327(18):1272-8, 1992
Kozarich et al., J. Biol. Chem., 252:7525, 1977
Kurman, et al., JAMA, 271:1866-1869, 1994.
Kurman, et al., The Bethesda system for reporting cervical/vaginal cytologic diagnoses. Definitions, criteria and explanatory notes for terminology and specimen adequacy. New York: Springer-Verlag. pp, 30-43, 1994.
Kuroda, et al., Lancet, 357:1225-1240, 2001.
Kwack, et al., Mol Cells, 10(5):575-8, 2000.
LaFace et al, Viology, 162:483-486, 1988.
Laughlin et al., J. Virol., 60:515-524, 1986.
Lawrence, et al., J. Biol. Chem, 245:3653, 1970.
Lebkowski, et al., Mol. Cell. Biol., 8:3988-3996, 1988.
Lipmann, et al., J. Biol. Chem. 161:415, 1945
Liu et al., Biochim Biophys Acta, 1240(2):277-84, 1995.
Lorenzato et al., J. Pathol., 194(2):171-6, 2001.
Lorincz, et al., Obstet. Gynecol., 79: 328-337, 1992.
Lukacher, et al., J Exp Med., 160(3):814-26, 1984.
Luo et al., Blood, 82:suppl. 1:303A, 1994.
Manning, et al., Biotechniques, 6:682, 1988.
Martin et al., Nature, 345(6277):739-743, 1990.
Matayoshi, et al., Science, 247:954, 1989
Matsuhashi et al., Proc. Natl. Acad. Sci. USA 54:587-594, 1965.
Mayer et al., Biochimica et Biophysica Acta, vol. 858, pp. 161-168, 1986.
Mayhew et al., Biochimica et Biophysica Acta, vol. 775, pp. 169-174, 1984.
Mayhew et al., Methods in Enzymology, vol. 149, pp. 64-77, 1987.
Mazmanian and Schneewind, In A. Sonenshine, Losick, and Hoch (ed.), Bacillus subtilis and other Gram-positive bacteria, 2 nd ed. ASM Press, New York, 2001.
Mazmanian et al., Mol. Microbiol, 2000.
Mazmanian et al., Proc. Natl. Acad. Sci. USA, 97:5510-5515, 2000.
Mazmanian et al., Science, 285:760-763, 1999.
Mazmanian et al., Mol. Microbiol. 40:1049-1057, 2001.
Mbvitz, Eur. J. Biochem. 68, 291-299, 1976
McCarty et al., J. Virol., 65:2936-2945, 1991.
McDevitt et al., Mol. Microbiol., 11:237-248, 1994.
McLaughlin et al., J. Virol., 62:1963-1973, 1988.
Mitchell, et al., Ann N Y Acad Sci., 690:153-66, 1993.
Mitchell, et al., J Clin Oncol., 8(5):856-69, 1990.
Moroney et al., J. Bioenerget. Biomembr, 14:347-359, 1982.
Morrison, et al., Int J Cancer. 49(1):6-13, 1991
Morton, et al., Ann Surg., 216(4):463-82, 1992.
Morton, et al., CA Cancer J. Clin., 46(4):225-44, 1996.
Munger, et al., J. Virol., 63: 4417-4421, 1989.
Munoz, et al., Int. J. Cancer, 52: 743-749, 1992.
Muzyczka, N., Curr. Top. Microbiol. Immunol., 158:97-129, 1992.
Nakagawa, et al., Clin. Diag. Lab. Immunol., 3: 205-210, 1996.
Nakagawa, et al., J. Infect. Dis., 175: 927-931, 1997.
Navarre et al., J. Bacteriol., 178:441-446, 1996.
Navarre et al., J. Biol. Chem., 273:29135-29142, 1998.
Navarre et al., J. Biol. Chem., 274. in press, 1999
Navarre et al., Microbiol. Mol. Biol. Rev., 63:174-229, 1999.
Navarre et al., Mol. Microbiol., 14:115-121, 1994.
Nehete et al., J. Clin. Immunol., 16:115-124, 1996.
Nehete, et al., Cell Immunol., 160(2):217-23, 1995.
Ni Eidhin et al., Mol. Microbiol., 30:245-257, 1998.
Nicolas, et al., In: Vectors: A survey of molecular cloning vectors and their uses, Rodriguez and Denhardt, eds., Butterworth, Stoneham, England, pp. 494-513, 1988.
Nicolau et al., Biochim. Biophys. Acta, 721:185-190, 1982.
Novick, Meth. Enzymol., 204:587-636, 1991.
Ohi et al., Gene, 89:279-282, 1990.
Oliver et al., Proc. Natl. Acad. Sci. USA, 87:8227-8231, 1990
Pallen et al., Trends Microbiol., 9:97-101, 2001.
Park, et al., Asia-Oceania J. Obstet. Gynaecol., 18: 171-175, 1992.
Parkin, et al., Int. J. Cancer, 84:827-841, 1999.
Paskind et al., Virology, 67:242-248, 1975.
Pathak et al., Biochemistry, 34:4179, 1995
Patti et al., Infect. Immun., 62:152-161, 1994.
Patti et al., J. Biol. Chem., 267:4766-4772, 1992.
Perbal, “A Practical Guide to Molecular Cloning” (2d ed., John Wiley & Sons, New York 1988).
Plenum Press, New York, pp. 117-148, 1986.
Poijak, et al., J. Clin. Microbiol. 37:796-797, 1999.
Potter et al., Proc. Nat'l Acad. Sci. USA, 81:7161-7165, 1984.
Powers, “Proteolytic Enzymes and Their Active-Site-Specific Inhibitors: Rose in the Treatment of Disease”
Pratt et al., Science, 246:917-919, 1989.
Ravindranath, et al., Int Rev Immunol, 7(4):303-29, 1991.
Reesei et al., Proc. Natl. Acad. Sci. USA 84:1127-1131 (1987).
Ridgeway, In: Vectors: A survey of molecular cloning vectors and their uses, Rodriguez R. L., Denhardt D. T., eds., Butterworth, Stoneham, England, pp. 467-492, 1988.
Rippe et al., Mol. Cell Biol., 10:689-695, 1990.
Rosenberg, et al., N Engl J. Med., 319(25):1676-80, 1988.
Rossen, et al., J Immunol., 135(5):3289-97, 1985.
Roux et al., Proc. Nat'l Acad. Sci. USA, 86:9079-9083, 1989.
Sambrook et al., “Molecular Cloning: A Laboratory Manual” (2d ed., Cold Spring Harbor Laboratory Press, Cold Spring Harbor, N.Y., 1988).
Sambrook, et al., Molecular Cloning, A Laboratory Manual, 2nd ed., Cold Spring Harbor Laboratory, Cold Spring, Harbor, N.Y., 1989.
Samulski et al., EMBO J., 10:3941-3950, 1991.
Samulski et al., J. Virol., 63:3822-3828, 1989.
Sarkar, et al., Viral Immunol., 8: 165-174, 1995.
Sastry, et al., Vaccine., 12(14): 1281-7, 1994.
Sastry, et al., Virology, 188: 502-509, 1992.
Schindler et al., Proc. Natl. Acad. Sci. USA, 51:414-421, 1964.
Schleifer et al., Bacteriol Rev., 36:407-477, 1972.
Schneewind et al., Cell, 70:267-281, 1992.
Schneewind et al., EMBO, 12:4803-4811, 1993.
Schneewind et al., Science, 268:103-106, 1995.
Seedorf, et al., EMBO J., 6: 139-144, 1987.
Shaw, “Cysteinyl Proteases and Their Selective Inactivation,” pp. 271-347.
Shelling, et al., Gene Therapy, 1:165-169, 1994.
Shepherd, et al., J. Gen. Virol., 73: 1269-1274, 1992.
Shinoda et al., Colloidal Surfactant, Academic Press, especially “The Formation of Micelles”, Ch. 1, 1-96, 1963.
Smith et al., Biochemistry, 14:766-771, 1975.
Smith et al., Gene, 67:31-40, 1988.
Solodin et al., Biochemistry, 34(41):13537-44, 1995.
Spanjer et al., Biochim Biophys Acta, 734(1):40-7, 1983.
Stauss, et al., Proc. Natl. Acad. Sci. USA, 89: 7871-7875, 1992.
Strang, et al., J. Gen. Virol., 71: 423-431, 1990.
Stratford-Perricaudet, et al., In: Human Gene Transfer, O. Cohen-Haguenauer and M. Boiron, eds., John Libbey Eurotext, France, p. 51-61, 1991.
Szoka, et al., Proc. Natl. Acad. Sci., 75:4194-4198, 1978.
Tam, et al., J. Am. Chem. Soc., 105:6442, 1983.
Tatusova et al., FEMS Microbiol. Lett., 174:247-250, 1999.
Templeton et al., Nat. Biotechnol., 15(7):647-52, 1997.
Thierry et al., Proc Natl Acad Sci USA., 92(21):9742-6, 1995.
Tijssen, “Practice and Theory of Enzyme Immunoassays” (Elsevier, Amsterdam, 1985), pp. 221-295.
Tindle, et al., Proc. Natl. Acad. Sci. USA, 88: 5887-5891, 1991.
Tobery, et al., J. Immunol. Methods, 254(1-2)59-66, 2001.
Ton-That et al., J. Biol. Chem., 272:22285-22292, 1997.
Ton-That et al., J. Biol. Chem., 273:29143-29149, 1998.
Ton-That et al., J. Biol. Chem., 274:24316-24320, 1999.
Ton-That et al., J. Biol. Chem., 275:9876-9881, 2000.
Ton-That et al., Proc. Natl. Acad. Sci. USA, 96:12424-12429, 1999.
Ton-That et al., Trends Microbiol, 9:101-102, 2001.
Tooze, J., ed., Molecular Biology of DNA Tumor Viruses, 2nd ed., Cold Spring Harbor Laboratory, Cold Spring Harbor, N.Y., 1991.
Townsend, et al., Cell, 44:949-968, 1986.
Tratschin et al., Mol. Cell. Biol., 4:2072-2081, 1984.
Tratschin et al., Mol. Cell. Biol., 5:32581-3260, 1985.
Tsukui, et al., Cancer Res., 56: 3967-3974, 1996.
Tur-Kaspa et al., Mol. Cell Biol., 6:716-718, 1986.
Uhlen et al., J. Biol. Chem. 259:1695-1702, 1984.
Unanue, et al., FASEB J., 13:2496-502, 1989.
Valentine et al., Am. J. Hematol., 11:111-124, 1981.
van de Rijn et al., Infect. Immun., 27:444-4.48, 1980
van de Rijn et al., Infect. Immun., 32:86-91, 1981
Von Knebel Doeberitz, et al., Cancer Res., 48: 3780-3786, 1988.
Wagner et al., Mol. Cell 40: 281-286, 1999.
Wallace, et al., Nucl. Acid Res., 9:879, 1981
Walsh et al., Proc. Nat'l Acad. Sci. USA, 89:7257-7261, 1994.
Wang et al., Nucl. Acids Res. 19:4000, 1991.
Wang et al., Tetrahedon Lett., 31:6493, 1990.
Wei et al., Gene Therapy, 1:261-268, 1994.
Westerik et al., J. Biol. Chem., 247:8195-8197, 1972.
Wettstein, et al., In: H. Pfister (ed.), Papillomaviruses and Human Cancer, pp. 145. Florida: CRC Press, 1990.
Wong, “Chemistry of Protein Conjugation and Cross-Linking” (CRC Press, Inc., Boca Raton, Fla. 1993).
Wu, et al., Biochemistry, 27:887-892, 1988.
Wu, et al., J. Biol. Chem., 262:4429-4432, 1987.
Xiong et al., Microbiology, 146:659-668, 2000
Yang et al., J. Virol., 68:4847-4856, 1994.
Yeung et al., J. Bacteriol., 172:2462, 1990
Yeung et al., J. Bacteriol., 66:1482, 1998
Yoder et al., Blood, 82:suppl. 1:347A, 1994.
Yokogawa et al., Antimicrob. Agents Chemother., 6:156-165, 1974.
Zhou et al., Exp. Hematol. (NY), 21:928-933, 1993.
Zhou et al., J. Exp. Med., 179:1867-1875, 1994.
Zhu et al., Chin J Biotechnol, 9(4):257-61, 1993.
Zubay, J. Mol. Biol. 4:347-356, 1962).
Zur Hausen, et al.,

What is claimed is:

1. A method for screening for a sortase-transamidase inhibitor comprising:
a) incubating at least a first sortase-transamidase and at least a first polypeptide comprising a sorting signal of NPQ/KTN/G, under conditions to allow the first sortase-transamidase to cleave the first polypeptide within the sorting signal;
b) incubating the first sortase-transamidase or the first polypeptide with a candidate inhibitor; and
c) assaying for activity of the first sortase-transamidase, wherein a reduction in activity identifies the compound as a sortase-transamidase inhibitor.

2. The method of claim 1 wherein the sorting signal is at the carboxyl-terminal end of the first polypeptide.

3. The method of claim 2, further comprising incubating the first sortase-transamidase, the first polypeptide, or the candidate compound with a Gram-positive bacterium having a peptidoglycan prior to assaying the activity of the first sortase-transamidase.

4. The method of claim 2, further comprising comparing the activity of the first sortase-transamidase with a sortase-transamidase not incubated with the candidate inhibitor.

5. The method of claim 2, wherein the first sortase-transamidase is srt B.

6. The method of claim 5, wherein the srt B is from Staphylococcus aureus.

7. The method of claim 6, wherein the srt B comprises SEQ ID NO:4.

8. The method of claim 2, wherein the first sortase-transamidase is substantially purified.

9. The method of claim 1, wherein the first sortase-transamidase is recombinant.

10. The method of claim 1, wherein the first polypeptide is recombinant.

11. The method of claim 2, wherein the first polypeptide comprises 6 contiguous amino acids from SEQ ID NO:33.

12. The method of claim 1, further comprising:
d) incubating the candidate inhibitor with a second sortase-transamidase and a second polypeptide comprising a sorting signal of LPX₃X₄G, under conditions to allow the second sortase-transamidase to cleave the second polypeptide within the sorting signal; and
e) assaying for activity of the second sortase-transamidase, wherein a reduction in activity identifies the candidate as a sortase-transamidase inhibitor.

13. The method of claim 12 wherein the sorting signal is at the carboxy-terminal end of the second polypeptide.

14. The method of claim 13 wherein the second polypeptide is recombinant.

15. The method of claim 14, wherein the second recombinant polypeptide further comprises a substantially hydrophobic domain of at least 31 amino acids carboxyl to the motif and a charged tail region with at least two positively charged residues carboxyl to the substantially hydrophobic domain, wherein at least one of the positively charged residues is an arginine and the two positively charged residues are located at residues 31-33 from the motif.

16. The method of claim 14, further comprising incubating the second sortase-transamidase, the second recombinant polypeptide, or the candidate compound with a Gram-positive bacterium having a peptidoglycan prior to assaying the activity of the second sortase-transamidase.

17. The method of claim 14, wherein the second recombinant polypeptide comprises at least 5 contiguous amino acids from SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31 or SEQ ID NO:32.

18. The method of claim 17, wherein the second recombinant polypeptide comprises at least 10 contiguous amino acids from SEQ ID NO:7, SEQ ID NO:8, SEQ ID NO:9, SEQ ID NO:10, SEQ ID NO:1, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31 or SEQ ID NO:32.

19. The method of claim 14, wherein the second sortase-transamidase is srt A.

20. The method of claim 19, wherein the srt A is from Staphylococcus aureus.

21. The method of claim 20, wherein the srt A comprises SEQ ID NO:2.

22. The method of claim 14, wherein the second sortase-transamidase is substantially purified.

23. The method of claim 14, wherein the second sortase-transamidase is recombinant.

24. The method of claim 14, wherein the second sortase-transamidase is obtained from a different species of Gram-positive bacterium than the first sortase transamidase.

25. The method of claim 1, wherein the first sortase-transamidase, the Gram-positive bacterium, or the first polypeptide is attached to a non-reacting structure.

26. The method of claim 1, wherein the first polypeptide further comprises a label.

27. The method of claim 26, wherein the label is radioactive, calorimetric, or enzymatic.

28. The method of claim 1, wherein the activity of the sortase-transamidase is assayed by measuring the amount of cleaved polypeptide.

29. The method of claim 3, wherein the activity of the sortase-transamidase is assayed by measuring the amount of the first polypeptide displayed on the surface of the bacterium.

30. The method of claim 29, wherein the amount of the first recombinant polypeptide displayed on the surface of the bacterium is measured using a proteinaceous compound that specifically binds the first recombinant polypeptide.

31. The method of claim 30, wherein the proteinaceous compound is attached to a label.

32. The method of claim 31, wherein the label is radioactive, calorimetric, or enzymatic.

33. The method of claim 14, further comprising:
d) incubating the candidate inhibitor with a third sortase-transamidase and a third polypeptide comprising a sorting signal, under conditions to allow the third sortase-transamidase to cleave the third polypeptide within the sorting signal; and
e) assaying for activity of the third sortase-transamidase, wherein a reduction in activity identifies the candidate as a sortase-transamidase inhibitor.

34. The method of claim 33 wherein the third polypeptide is recombinant.

35. The method of claim 34, wherein the third sortase-transamidase is obtained from a different species of Gram-positive bacterium than the first and second sortase transamidases.

36. The method of claim 1, further comprising:
d) administering to an animal a Gram-positive bacterium;
b) administering to the animal the candidate inhibitor;
c) assaying the pathogenicity of the bacterium.

37. The method of claim 36, wherein the Gram-positive bacterium is Staphylococcus aureus.

38. The method of claim 36, further comprising comparing the pathogenicity of the bacteria in the animal with a second animal administered a Gram-positive bacterium having a mutation in srt A, srt B, or both.

39. The method of claim 36, wherein the animal is a mouse.

40. The method of claim 36, wherein the pathogenicity of the bacterium is assayed by measuring an amount of bacterium in the animal.

41. The method of claim 36, wherein the pathogenicity of the bacterium is assayed by measuring abscess formation.

42. The method of claim 36, wherein the candidate compound is administered to the animal more than 24 hours after administration of the bacterium to the animal.

43. A method of screening for a sortase-transamidase inhibitor comprising:
a) administering to an animal a Gram-positive bacteriun having a mutation in at least one sortase-transamidase-encoding nucleic acid, wherein the bacterium does not express a functional sortase-transamidase encoded by the nucleic acid;
b) administering to the animal a candidate compound;
c) assaying the pathogenicity of the bacterium.

44. A method for screening for expression of a recombinant polypeptide comprising:
a) incubating a sortase-transamidase, a Gram-positive bacterium having a peptidoglycan, and a polypeptide comprising a sorting signal of NPQ/KTN/G, under conditions to allow the sortase-transamidase to cleave the polypeptide within the sorting signal; and
b) assaying for display of the polypeptide on the surface of the Gram-positive bacterium.

45. The method of claim 44 wherein the sorting signal is at the carboxyl terminal end of the polypeptide.

46. The method of claim 45 wherein the polypeptide is recombinant.

47. The method of claim 46, wherein the sortase-transamidase is srt B.

48. The method of claim 47, wherein the srt B is from Staphylococcus aureus.

49. The method of claim 48, wherein the srt B comprises SEQ ID NO:4.

50. The method of claim 46, wherein the sortase-transamidase is substantially purified.

51. The method of claim 46, wherein the sortase-transamidase is recombinant.

52. The method of claim 46, wherein the polypeptide comprises 6 amino acids of SEQ ID NO:33.

53. The method of claim 46, wherein the polypeptide is assayed using a proteinaceous compound that specifically binds the polypeptide.

54. The method of claim 53, wherein the proteinaceous compound is labeled.

55. The method of claim 54, wherein the proteinaceous compound is labeled with an enzymatic marker, a colorimetric marker, or a radiolabel.

56. The method of claim 53, wherein the proteinaceous compound is an antibody.

57. The method of claim 53, wherein the proteinaceous compound is a ligand for the polypeptide.

58. The method of claim 53, wherein the proteinaceous compound is a substrate for the polypeptide.

59. A method of diagnosing or treating a Gram-positive bacteria infection in an organism comprising:
a) linking an antibiotic or a detection reagent to a polypeptide comprising a sorting signal of NPQ/KTN/G; and
b) introducing the linked polypeptide to the organism, wherein the linked polypeptide becomes covalently cross-linked to the bacterium.

60. The method of claim 59 wherein the sorting signal is at the carboxyl terminal end of the polypeptide.

61. The method of claim 60, wherein the antibiotic or detection reagent is conjugated to the polypeptide.

62. A conjugate comprising an antibiotic or detection reagent conjugated to a polypeptide comprising a sorting signal of NPQ/KTN/G at its carboxyl-terminal end.

63. A recombinant cleaved polypeptide displayed on the surface of a Gram-positive bacterium comprising a sortase-transamidase by covalent linkage, wherein the polypeptide comprised an amino acid sequence of NPQ/KTN/G prior to being cleaved by the sortase-transamidase.

64. The recombinant cleaved polypeptide of claim 63, wherein the bacterium is Staphylococcus aureus.

65. A vaccine comprising a Gram-positive bacterium displaying a recombinant cleaved polypeptide on its surface, wherein the polypeptide is covalently attached to the bacterium and comprises a sorting sequence comprising an amino acid sequence of NPQ/KTN/G or LPX₃X₄G prior to being cleaved by a sortase-transamidase in the bacterium.

66. The vaccine of claim 65, wherein the polypeptide comprises a sorting sequence comprising an amino acid sequence of NPQ/KTN/G prior to being cleaved by a sortase-transamidase in the bacterium.

67. The vaccine of claim 66, wherein the polypeptide comprises 6 contiguous amino acids from SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, or SEQ ID NO:33.

68. The vaccine of claim 65, wherein the Gram-positive bacterium is Staphylococcus aureus.

69. The method of claim 65, wherein the Gram-positive bacterium comprises a genetic mutation, wherein the mutation reduces the pathogenicity of the bacterium compared to a bacterium lacking the mutation.

70. A method for inducing an immune response in an animal comprising administering to the animal the vaccine of claim 65 in an amount effective to induce an immune response against the antigen.

71. A method of making a vaccine comprising incubating a sortase-transamidase, a Gram-positive bacterium having a peptidoglycan, and a recombinant polypeptide comprising a sorting signal of NPQ/KTN/G at its carboxyl-terminal end and an antigen, under conditions to allow the sortase-transamidase to cleave the polypeptide within the sorting signal.

72. The method of claim 71, wherein the antigen is a tumor-associated antigen or a virus antigen or a bacterial antigen.

73. The method of claim 71, wherein the Gram-positive bacterium comprises a genetic mutation, wherein the mutation reduces the pathogenicity of the bacterium compared to a bacterium lacking the mutation.

74. The method of claim 71, wherein the polypeptide comprises 6 contiguous amino acids of SEQ ID NO:33.

75. A method for identifying a sorting signal comprising:
a) searching a protein database for homologous amino acid sequences in Gram-positive bacterium using a query lacking the sequence of LPX₃X₄G and NPQTN;
b) identifying at least a first protein sequence that has at least 10% identity or 20% homology to a second protein sequence;
c) obtaining a polypeptide comprising an identified protein sequence; and
d) incubating the polypeptide with a sortase-transamidase under conditions to allow the polypeptide to be cleaved.
